(19) United States
(12) Patent Application Publication
(10) Pub. No.: US 2025/0011824 A1
(43) Pub. Date: Jan. 9, 2025

(54) METHOD TO SYNTHESIZE CHITIN OLIGOSACCHARIDES

(71) Applicants: UNIVERSITEIT GENT, Gent (BE); INSTITUT QUÍMIC DE SARRIÀ, Barcelona (ES)

(72) Inventors: Marjan DE MEY, Gent (BE); Jo MAERTENS, Gent (BE); Chiara GUIDI, Gent (BE); Xevi BIARNES, Barcelona (ES); Antoni PLANAS, Barcelona (ES)

(21) Appl. No.: 18/710,905

(22) PCT Filed: Nov. 17, 2022
(86) PCT No.: PCT/EP2022/082232

§ 371 Date: May 16, 2024

(30) Foreign Application Priority Data
Nov. 18, 2021 (EP) 21208979.1

(51) Int. Cl. C12P 19/18; C12N 9/10; C12P 19/26

(52) U.S. Cl. CPC C12P 19/18; C12N 9/1051; C12P 19/26

(57) ABSTRACT

The present invention relates to mutated chitin oligosaccharide synthases and their usage to produce molecules having useful features. More specifically, the present invention discloses the usage of engineered microorganisms expressing a mutated chitin oligosaccharide synthase to produce chitin oligosaccharides having a degree of polymerization of four, five, six or seven.

### TECHNICAL FIELD OF INVENTION

The present invention relates to mutated chitin oligosaccharide synthases and their usage to produce molecules having useful features. More specifically, the present invention discloses the usage of engineered microorganisms expressing a mutated chitin oligosaccharide synthase to produce chitin oligosaccharides having a degree of polymerization of four, five, six or seven.

### BACKGROUND OF THE INVENTION

Chitin oligosaccharides (COS) (2<degree of polymerization (DP)<11) are oligomers of N-acetylglucosamine (GIcNAc) units (see FIG. 1). COS molecules are bioactive molecules with potential applications in the food (Rakkhumkaew and Pengsuk 2018; Rao, Chander, and Sharma 2008) and feed industry (Wan et al. 2017; Duan et al. 2020), cosmetics (Aranaz et al. 2018), and health care (Wolinsky, Colson, and Grinstaff 2012; Xu et al. 2020), and many of their bioactivities appear to reflect highly specific effector-receptor interactions (the lock-and-key theory) (Hayafune et al. 2014). For example, COS with DPs of 7-8 directly affect plants by increasing their tolerance to fungal and bacterial diseases (Hayafune et al. 2014; Basa et al. 2020; Wan et al. 2008; Feng et al. 2019). COS-derived products could also be developed for applications in medicine and drug delivery, e.g. increased ability to cross the blood-brain barrier (BBB), which could improve the efficiency of drugs targeting Alzheimer's disease (Yu et al. 2019; Ouyang et al. 2017; Akhlaghi, Berry, and Tam 2013; Ojeda-Hernandez et al. 2020; Fliegmann and Bono 2015). In addition to an expanded COS portfolio, industry would greatly benefit from the production of specific and well-defined COS, due to the opportunities for the feed market, e.g. accelerated piglet growth rate and improved piglet immune response (Aranaz et al. 2018).

Current technologies are not able to produce pure oligomers with fully defined architecture and in sufficient amounts to study their biological activities. The most common production technologies are typically based on random chemical and/or enzymatic depolymerization and deacetylation of chitin from the exoskeletons from crustacea but are thus seriously flawed by various disadvantages. Typically complex COS mixtures are obtained (Aam et al. 2010), necessitating extensive downstream processing efforts to purify the active product, which inflates the production cost enormously and hampers numerous applications in diverse sectors. In this respect, neither carbohydrate chemistry (Yang and Ding 2014), nor the enzymatic or chemical depolymerization of chitin and chitosan (Cabrera and Van Cutsem 2005) are fully apt to sustainably produce COS with a completely defined architecture. Whereas the first route is hampered by, i.a., the need for various protecting and de-protecting steps, a low conversion-efficiency, the generation of toxic waste and the use of expensive catalysts, the latter inherently necessitates extensive purification to separate the targeted COS from the obtained heterogeneous COS mixtures (Cabrera and Van Cutsem 2005). A possible solution to create COS with a defined DP in pure and high yield is the synthesis of COS instead of the break-down route.

Chitin synthases (CHSs) utilize the nucleotide sugar donor UDP-GlcNAc and transfer the α-linked GlcNAc sugar in an inverting mechanism onto the non-reducing end of the growing acceptor oligosaccharide. CHS enzymes are classified in the CAZy database as belonging to the GT-2 family. This family contains inverting glycosyl transferases (GTs) such as CHSs, cellulose synthases, and hyaluronan synthases. CHSs contain multiple transmembrane (TM) domains that are thought to form a transport channel for the deposition of chitin on the outer membrane, similar to cellulose synthases. The size and shape of the transport channel in enzymes such as cellulose or chitin synthases are important to prevent premature release of the glucan chain (McNamara, Morgan, and Zimmer 2015; Oehme et al. 2019). The size of the channel is decreased due to the presence of aromatic residues, enabling important C—H-π interactions and hydrogen bonds interactions (Morgan, McNamara, and Zimmer 2014; Knott et al. 2016; Oehme et al. 2019). Next, of particular interest is the well-conserved QxxRW sequence in several of these membrane-embedded β-glycosyltransferases. Up till now, it is believed that the QxxRW motif acts as product binding site, retaining it for further addition of monomers, such as GlcNAc or Glc (H. C. Dorfmueller et al. 2014; Morgan, Strumillo, and Zimmer 2013; Morgan, McNamara, and Zimmer 2014; Oehme et al. 2019; Kaur et al. 2016; Perez-Mendoza et al. 2017). Of specific importance is Trp383 (W in the QxxRW motif), enabling a strong carbohydrate-n stacking interaction and stabilizing translocation.

Chitin oligosaccharide synthases (NodCs) from different Rhizobial species synthesizes only short, soluble chitooligosaccharides, varying from (GlcNAc)3 up to (GlcNAc)5 and in minor amounts (GlcNAc)6 (Leppyanen et al. 2014), whereas CHS enzymes produce long, insoluble polysaccharides that are deposited in the cell wall. For example, NodC from S. meliloti (SmNodC) predominantly yields (GlcNAc)4, while NodC from R. sp. GRH2 (RsNodC) predominantly yields (GlcNAc)5. However, no NodCs are described which yields COS with a predominant DP of more than 5. Next, the native NodC(-like) enzymes always produce a COS-mixture with two or more different DPs (Leppyanen et al. 2014; Kamst and Breek 2000). NodC proteins are smaller than CHS enzymes (^{˜}420 amino acids in length compared with 900 to several thousand amino acids in length) because NodC enzymes lack two domains observed in chitin synthases: the N-terminal domain and the C-terminal transmembrane domain that is predicted to form a chitin transport channel across the membrane.

Until now, no crystal structure is available for neither CHS nor NodC(-like) enzymes, making protein engineering of NodC to yield COS with higher or specific DP very challenging. Hence, the correlation between NodC structure and DP is therefore unknown. A previous study with chimeras of NodC enzymes from different species identified that the C-terminus beyond Ile-262 regulates COS product ratio between DP4 and DP5 (Kamst and Breek 2000). In order to study CHS enzymes on a structural and mechanistic level, Dorfmueller et al. (2014) (H. C. Dorfmueller et al. 2014) created a homology model of the NodC from S. meliloti (SmNodC) on the basis of the published BcsA cellulose synthase structure.

Homology modelling, also known as comparative modelling of proteins, refers to constructing an atomic-resolution model of the “target” protein from its amino acid sequence and an experimental three-dimensional structure of a related homologous protein (the “template”). Homology modelling relies on the identification of one or more known protein structures likely to resemble the structure of the query sequence, and on the production of an alignment that maps residues in the query sequence to residues in the template sequence. Using the model of Dorfmueller et al. (2014) (H. C. Dorfmueller et al. 2014), a reaction mechanism for NodC and CHS was proposed: the 1-hydroxyl group of the donor substrate UDP-GIcNAc is transferred onto the non-reducing end of the growing acceptor oligosaccharide. Furthermore, they hypothesized that the predicted product-binding site for SmNodC is limited by two amino acids, Arg-349 and Leu-19, to five binding sites, whereas the cellulose synthase structure forms a transport channel through the membrane formed by the transmembrane domains (Ser-111 and Ser-459). This suggests that increasing space in the catalytic cleft towards the formation of a transport channel through the membrane could increase the backbone length. They further suggest that to obtain such a channel, you should shorten the bulky side chains of amino acids sterically blocking the growth of a COS transportation channel.

The present invention overcomes the above-described disadvantages as it provides engineered chitin oligosaccharide synthases which yield a mixture of COS in which the percentages of the fractions of the chitin oligosaccharides with higher DP is increased compared to the wild-type enzyme. This is accomplished by site-directed engineering of the enzyme activity/activities converting the nucleotide sugar UDP-GIcNAc to COS with higher DP and specifically controlling the amount of elongation rounds to yield COS with a particular DP.

### BRIEF DESCRIPTION OF FIGURES

FIG. 1. Overview of the chitin oligosaccharides synthesized using the engineering strategy described in this invention. m=3 to 10

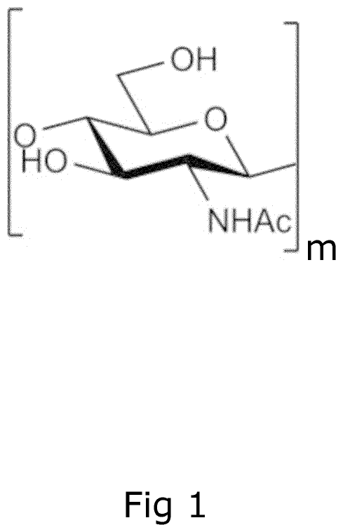

FIG. 2. Schematic representation of the protein engineering strategy of chitin oligosaccharide synthases to synthesize chitin oligosaccharides with a controllable chain length. (a) Example in which a positive charged amino acid in the transmembrane region 1 is replaced by an amino acid with the opposite charge (negative charged amino acid) or a neutral amino acid (uncharged amino acid) yielding a chitin oligosaccharide product portfolio with increased fraction of chitin oligosaccharides with higher degree of polymerization. Protein is depicted in front view. (b) Example in which a positive charged amino acid in the transmembrane region 2 is replaced by an amino acid with the opposite charge (negative charged amino acid) or a neutral amino acid (uncharged amino acid) yielding a chitin oligosaccharide product portfolio with increased product specificity. Protein rotated over 180°.

FIG. 3. Chromatogram of chitin oligosaccharides (COS) production in strain Escherichia coli sSmNodC1 at 24 h: E. coli 3KO+pCOSA4-SmNodC serves as positive control with maximum peaks at (GlcNAc)4 and (GlcNAc)5. Visualized using OpenChrom. GlcNAc=A=N-acetyl glucosamine.

FIG. 4. Chromatogram of chitin oligosaccharides (COS) production in strain Escherichia coli sSmNodC2 at 24 h: E. coli 3KO+pCOSA4-SmNodC R349S. Visualized using OpenChrom. GlcNAc=A=N-acetyl glucosamine.

FIG. 5. Chitin oligosaccharides production profile in strains Escherichia coli sSmNodC1 (WT), sSmNodC2 (R349S), sSmNodC3 (R349E) and sSmNodC5 (H356R) at 24 h. A=N-acetyl glucosamine.

FIG. 6. Chromatogram of chitin oligosaccharides (COS) production in strain Escherichia coli sSmNodC3 at 24 h: E. coli 3KO+pCOSA4-SmNodC R349E. Visualized using OpenChrom. GlcNAc=A=N-acetyl glucosamine.

FIG. 7. Chromatogram of chitin oligosaccharides (COS) production in strain Escherichia coli sRsNodC1 at 24 h: E. coli 3KO+pCOSA5-RsNodC serves as positive control with maximum peak at (GlcNAc)5. Visualized using OpenChrom. GlcNAc=A=N-acetyl glucosamine.

FIG. 8. Chromatogram of chitin oligosaccharides (COS) production in strain Escherichia coli sRsNodC2 at 24 h: E. coli 3KO+pCOSA5-RsNodC R346D. Visualized using OpenChrom. GlcNAc=A=N-acetyl glucosamine.

FIG. 9. Chromatogram of chitin oligosaccharides (COS) production in strain Escherichia coli sRsNodC3 at 24 h: E. coli 3KO+pCOSA5-RsNodC R346E. Visualized using OpenChrom. GlcNAc=A=N-acetyl glucosamine.

FIG. 10. Chromatogram of chitin oligosaccharides (COS) production in strain Escherichia coli sRsNodC4 at 24 h: E. coli 3KO+pCOSA5-RsNodC R353E. Visualized using OpenChrom. GlcNAc=A=N-acetyl glucosamine.

FIG. 11. Chromatogram of chitin oligosaccharides (COS) production in strain Escherichia coli sRsNodC5 at 24 h: E. coli 3KO+pCOSA5-RsNodC R355E. Visualized using OpenChrom. GlcNAc=A=N-acetyl glucosamine.

FIG. 12. Chromatogram of chitin oligosaccharides (COS) production in strain Escherichia coli sRsNodC6 at 24 h: E. coli 3KO+pCOSA5-RsNodC R358E. Visualized using OpenChrom. GlcNAc=A=N-acetyl glucosamine.

FIG. 13. Chitin oligosaccharides production profile in strains Escherichia coli sRsNodC1 (WT), sRsNodC2 (R346D), sRsNodC3 (R346E), sRsNodC4 (R353E), sRsNodC5 (R355E) and sRsNodC6 (R358E) at 24 h. A=N-acetyl glucosamine.

FIG. 14. Chromatogram of chitin oligosaccharides (COS) production in strain Escherichia coli sSfNodC1 at 24 h: E. coli 3KO+pCOSA5-SfNodC. Visualized using OpenChrom. GlcNAc=A=N-acetyl glucosamine.

FIG. 15. Chromatogram of chitin oligosaccharides (COS) production in strains Escherichia coli sSfNodC2 at 24 h: E. coli 3KO+pCOSA5-SfNodC R349E. Visualized using OpenChrom. GlcNAc=A=N-acetyl glucosamine.

FIG. 16. Chitin oligosaccharides production profile in strains Escherichia coli sSfNodC1 (WT), sSfNodC2 (R349E) and sSfNodC3 (R349S) at 24 h. A=N-acetyl glucosamine.

FIG. 17. Chromatogram of chitin oligosaccharides (COS) production in strain Escherichia coli sRsNodC7 at 24 h: E. coli 3KO+pCOSA5-RsNodC R346E-R353E. Visualized using OpenChrom. GlcNAc=A=N-acetyl glucosamine.

FIG. 18. Chromatogram of chitin oligosaccharides (COS) production in strain Escherichia coli sRsNodC8 at 24 h: E. coli 3KO+pCOSA5-RsNodC R346E-R355E. Visualized using OpenChrom. GlcNAc=A=N-acetyl glucosamine.

FIG. 19. Chromatogram of chitin oligosaccharides (COS) production in strain Escherichia coli sRsNodC9 at 24 h: E. coli 3KO+pCOSA5-RsNodC R346E-R358E. Visualized using OpenChrom. GlcNAc=A=N-acetyl glucosamine.

FIG. 20. Chromatogram of chitin oligosaccharides (COS) production in strain Escherichia coli sRsNodC10 at 24 h: E. coli 3KO+pCOSA5-RsNodC R197S-R346E. Visualized using OpenChrom. GlcNAc=A=N-acetyl glucosamine.

FIG. 21. Chromatogram of chitin oligosaccharides (COS) production in strain Escherichia coli sRsNodC11 at 24 h: E. coli 3KO+pCOSA5-RsNodC R346E-R358S. Visualized using OpenChrom. GlcNAc=A=N-acetyl glucosamine.

FIG. 22. Chromatogram of chitin oligosaccharides (COS) production in strain Escherichia coli sRsNodC12 at 24 h: E. coli 3KO+pCOSA5-RsNodC H298T-R346E. Visualized using OpenChrom. GlcNAc=A=N-acetyl glucosamine.

FIG. 23. Chitin oligosaccharides production profile in strains Escherichia coli sRsNodC1 (WT) and sRsNodC7 (R346E-R353E), sRsNodC8 (R346E-R355E), sRsNodC9 (R346E-R358E), sRsNodC10 (R197S-R346E), sRsNodC11 (R346E-R358S) and sRsNodC12 (H298T-R346E) at 24 h. A=N-acetyl glucosamine.

FIG. 24. Chromatogram of chitin oligosaccharides (COS) production in strain Escherichia coli sRsNodC13 at 24 h: E. coli 3KO+pCOSA5-RsNodC M342S-R346E. Visualized using OpenChrom. GlcNAc=A=N-acetyl glucosamine.

FIG. 25. Chitin oligosaccharides production profile in strains Escherichia coli sRsNodC1 (WT) and RsNodC13 (M342S-R346E) at 24 h. A=N-acetyl glucosamine.

FIG. 26. Chromatogram of chitin oligosaccharides (COS) production in strain Escherichia coli sRsNodC14 at 24 h: E. coli 3KO+pCOSA5-RsNodC R197S. Visualized using OpenChrom. GlcNAc=A=N-acetyl glucosamine.

FIG. 27. Chromatogram of chitin oligosaccharides (COS) production in strain Escherichia coli sRsNodC15 at 24 h: E. coli 3KO+pCOSA5-RsNodC R346S. Visualized using OpenChrom. GlcNAc=A=N-acetyl glucosamine.

FIG. 28. Chromatogram of chitin oligosaccharides (COS) production in strain Escherichia coli sRsNodC16 at 24 h: E. coli 3KO+pCOSA5-RsNodC R346Q. Visualized using OpenChrom. GlcNAc=N-acetyl glucosamine.

FIG. 29. Chromatogram of chitin oligosaccharides (COS) production in strain Escherichia coli sRsNodC17 at 24 h: E. coli 3KO+pCOSA5-RsNodC R346W. Visualized using OpenChrom. GlcNAc=A=N-acetyl glucosamine.

FIG. 30. Chitin oligosaccharides production profile in strains Escherichia coli sRsNodC1 (WT), sRsNodC14 (R197S), sRsNodC15 (R346S), sRsNodC16 (R346Q) and sRsNodC17 (R346W) at 24 h. A=N-acetyl glucosamine.

FIG. 31. Chromatogram of chitin oligosaccharides (COS) production in strain Escherichia coli sSmNodC4 at 24 h: E. coli 3KO+pCOSA4-SmNodC R200S. Visualized using OpenChrom. GlcNAc=A=N-acetyl glucosamine.

FIG. 32. Chitin oligosaccharides production profile in strains Escherichia coli sSmNodC1 (WT) and sSmNodC4 (R200S) and sSmNodC6 (R200S-R349E) at 24 h. A=N-acetyl glucosamine.

FIG. 33. Chromatogram of chitin oligosaccharides (COS) production in strain Escherichia coli sRsNodC18 at 24 h: E. coli 3KO+pCOSA5-RsNodC H298T-R346S. Visualized using OpenChrom. GlcNAc=A=N-acetyl glucosamine.

FIG. 34. Chitin oligosaccharides production profile in strains Escherichia coli sRsNodC1 (WT), sRsNodC10 (R197S-R346E), sRsNodC11 (R346E-R358S) and sRsNodC18 (H298T-R346S) at 24 h. A=N-acetyl glucosamine.

FIG. 35. Chromatogram of chitin oligosaccharides (COS) production in strain Escherichia coli sRsNodC19 at 24 h: E. coli 3KO+pCOSA5-RsNodC M342S-R346S. Visualized using OpenChrom. GlcNAc=A=N-acetyl glucosamine.

FIG. 36. Chromatogram of chitin oligosaccharides (COS) production in strain Escherichia coli sRsNodC20 at 24 h: E. coli 3KO+pCOSA5-RsNodC M342W-R346S. Visualized using OpenChrom. GlcNAc=A=N-acetyl glucosamine.

FIG. 37. Chitin oligosaccharides production profile in strains Escherichia coli sRsNodC1 (WT), sRsNodC19 (M342S-R346S) and sRsNodC20 (M342W-R346S) at 24 h. A=N-acetyl glucosamine.

FIG. 38. Chromatogram of chitin oligosaccharides (COS) production in strain Escherichia coli sRsNodC21 at 24 h: E. coli 3KO+pCOSA5-RsNodC H298T-L302T. Visualized using OpenChrom. GlcNAc=A=N-acetyl glucosamine.

FIG. 39. Chitin oligosaccharides production profile in strains Escherichia coli sRsNodC1 (WT) and sRsNodC21 (H298T-L302T) at 24 h. A=N-acetyl glucosamine.

FIG. 40. Chromatogram of chitin oligosaccharides (COS) production in strain Escherichia coli sSmNodC5 at 24 h: E. coli 3KO+pCOSA4-SmNodC H356R. Visualized in Openchrom. GlcNAc=A=N-acetyl glucosamine.

FIG. 41. Chromatogram of chitin oligosaccharides (COS) production in strain Escherichia coli sSmNodC6 at 24 h: E. coli 3KO+pCOSA4-SmNodC R200S-R349E. Visualized in Openchrom. GlcNAc=A=N-acetyl glucosamine.

FIG. 42. Chromatogram of chitin oligosaccharides (COS) production in strain Escherichia coli sSfNodC3 at 24 h: E. coli 3KO+pCOSA5-SfNodC R349S. Visualized using OpenChrom. GlcNAc=A=N-acetyl glucosamine.

### DESCRIPTION OF INVENTION

The present invention discloses mutated chitin oligosaccharide synthases and their usage to produce chitin oligosaccharides having a degree of polymerization of four, five, six and/or seven.

Hence, the present invention relates to usage of mutated, bacterial chitin oligosaccharide synthases to increase at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%, and, wherein said mutation is a substitution of at least one charged amino acid present in one of the two transmembrane helices of said chitin oligosaccharide synthase by an amino acid having the opposite charge or by an amino acid having no charge.

For example, the present invention discloses that wild type RsNodC (SEQ ID No 1) produces a mixture of 0% chitin oligosaccharides having a degree of polymerization equal to 4, 96% chitin oligosaccharides having a degree of polymerization equal to 5, 4% chitin oligosaccharides having a degree of polymerization equal to 6, and, 0% chitin oligosaccharides having a degree of polymerization equal to 7, whereas usage of a mutant of said RsNodC characterized by a positively charged amino acid arginine at amino acid position 346 substituted by a neutral or uncharged serine neutral or uncharged tryptophan (R346W) produces a mixture of 0% chitin oligosaccharides having a degree of polymerization equal to 4, 15% chitin oligosaccharides having a degree of polymerization equal to 5, 85% chitin oligosaccharides having a degree of polymerization equal to 6, and, 0% chitin oligosaccharides having a degree of polymerization equal to 7.

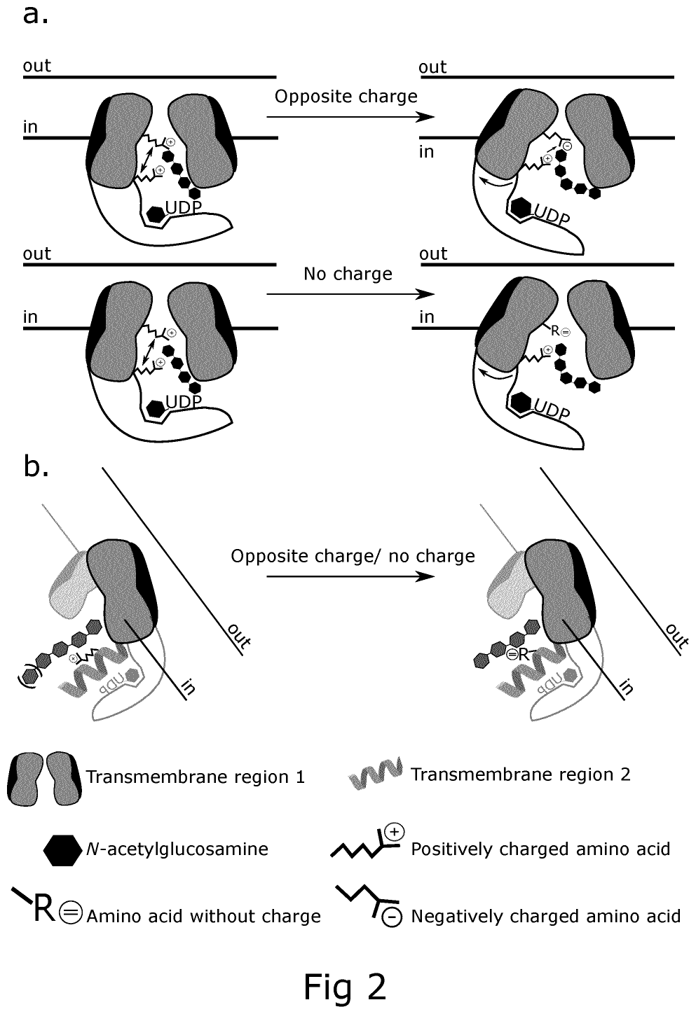

In other words, the present invention relates to a method to control the degree of polymerization of produced chitin oligosaccharides. The mutated chitin oligosaccharide synthase is derived using a specific protein engineering strategy which is based on the substitution of one (or more) charged amino acids in to any other opposite charged or neutral amino acid, regardless the bulkiness of the opposite charged or neutral amino acid. This substitution of one (or more) charged amino acid in to any other opposite charged or neutral amino acid induces a structural change in the transmembrane helices of the chitin oligosaccharide synthase which results in increased binding cavity. This increased binding cavity of the mutated chitin oligosaccharide synthase allows to control the fractions in the chitin oligosaccharide product profile (see FIG. 2). Controlling the fractions in the chitin oligosaccharide product portfolio does not require the creation of a transport channel through the membrane. For example, substituting a charged amino acid in the transmembrane region 1 of the chitin oligosaccharide synthase into any other opposite charged or neutral amino acid, regardless the bulkiness of the opposite charged or neutral amino acid, results in an increased fraction of chitin oligosaccharides with a higher degree of polymerization (see FIG. 2 a). Another example is the substitution of a charged amino acid in the transmembrane region 2 into any other opposite charged or neutral amino acid, regardless the bulkiness of the opposite charged or neutral amino acid, resulting in a chitin oligosaccharide product portfolio with increased product specificity (see FIG. 2 b).

Using the method of the present invention, product formation through the addition of UDP-GlcNAc to the GlcNAc acceptor can be controlled in terms of degree of polymerization.

This means that the former drawback of having a mixture of chitin oligosaccharides, which varies from batch-to-batch with conventional methods, is eliminated. The methodology results in specific chitin oligosaccharide production with an increased degree of polymerization.

The present invention thus relates to the production of chitin oligosaccharides consisting of fully acetylated N-acetylglucosamine residues having degrees of polymerization of four, five, six or seven.

The term ‘degree of polymerization’ refers to the number of β-1,4-linked N-acetylglucosamine residues.

The term ‘protein engineering’ refers to the practice of optimizing the catalytic mechanism of said enzyme to control the final degree of polymerization of said chitin oligosaccharides. To this end, any method known in the art which can be used to modify the amino acid sequence of a protein can be used. Examples of such methods are site-directed mutagenesis, error-prone PCR, SSA, CPEC as described in (Coussement et al. 2014; Ji. Quan and Tian 2014; Siloto and Weselake 2012; Fujii 2004; Cadwell and Joyce 1992; Bommarius, Blum, and Abrahamson 2011; Madhavan et al. 2021).

The term ‘charge’ refers to the characteristics of the side chains of amino acids: positively, negatively, polar uncharged or hydrophobic.

The term ‘positively charged amino acid’ relates to any amino acid having a positively charged side chain, i.e. arginine, lysine or histidine.

The term ‘negatively charged amino acid’ relates to any amino acid having a negatively charged side chain, i.e. aspartic acid or glutamic acid.

The term ‘neutral amino acid’ relates to any amino acid having a neutral side chain, i.e. serine, threonine, asparagine, glutamine, cysteine, selenocysteine, glycine, proline, alanine, valine, isoleucine, leucine, methionine, phenylalanine, tyrosine, tryptophan.

The term ‘chitin oligosaccharide synthase’ relates to any glycosyltransferase selected from but not limited to the group consisting of beta-polysaccharide synthase, chitin synthase (Dorfmueller et al. 2014), cellulose synthase (Morgan, McNamara, and Zimmer 2014; Morgan, Strumillo, and Zimmer 2013), N-acetylglucosaminyltransferase (Leppyanen et al. 2014), beta-1,4-N-acetylglucosaminyltransferase (Kamst et al. 1997), nodulation protein C (NodC) (Kamst et al. 1997), NodC-like enzyme (Semino and Robbins 1995), chitooligosaccharide synthase (Dorfmueller et al. 2014), hyaluronic acid synthase, hyaluronan synthase (Weigel et al. 2014) and glycosyl transferase family 2 (Bi et al. 2015).

Additionally, said enzymes with glycosyltransferase activity is encoded by a gene selected from—but not limited to—the group consisting of CHS, BcsA-B, NodC, nodBC, nodCB and hasA.

Additionally, said enzymes with glycosyltransferase activity is possibly (but not solely) originating from bacteria.

Additionally, said enzymes with glycosyltransferase activity is possibly (but not solely) originating from the bacterial genus Rhizobium, Sinorhizobium, Cupriavidus, Burkholderia, Corallococcus, Desulfobacterium, Actinobacteria, Methylobacteria, Microvirga, Brucella, Bosea, Bradyrhizobium, Ochrobactrum, Devosia, Aminobacter, Mesorhizobium, Phyllobacterium, Agrobacterium, Allorhizobium, Neorhizobium, Shinella, Azorhizobium, Paraburkholderia and Pseudomonas More specifically, said enzymes comprises an amino acid sequence given by but not limited to SEQ ID No 1 (=NodC of Rhizobium species strain GRH2), SEQ ID No 2 (=NodC of Sinorhizobium meliloti), SEQ ID No 3 (=NodC from Sinorhizobium fredii USDA 191).

More specifically, the present invention relates to the protein engineering of said enzymes with glycosyltransferase activity as indicated above resulting in a mutated enzyme with glycosyltransferase activity highly selective towards the formation of chitin oligosaccharides with an increased degree of polymerization compared to their wild-type activity.

More specifically, the present invention relates to the usage of a mutated, bacterial chitin oligosaccharide synthase as described above wherein said bacterial species belongs to the genus Rhizobium, Sinorhizobium, Cupriavidus, Burkholderia, Corallococcus, Desulfobacterium, Actinobacteria, Methylobacteria, Microvirga, Brucella, Bosea, Bradyrhizobium, Ochrobactrum, Devosia, Aminobacter, Mesorhizobium, Phyllobacterium, Agrobacterium, Allorhizobium, Neorhizobium, Shinella, Azorhizobium, Paraburkholderia or Pseudomonas.

More specifically, the present invention relates to the method to produce a mutated, bacterial chitin oligosaccharide synthase as described above wherein said bacterial species belongs to the genus Rhizobium, Sinorhizobium, Cupriavidus, Burkholderia, Corallococcus, Desulfobacterium, Actinobacteria, Methylobacteria, Microvirga, Brucella, Bosea, Bradyrhizobium, Ochrobactrum, Devosia, Aminobacter, Mesorhizobium, Phyllobacterium, Agrobacterium, Allorhizobium, Neorhizobium, Shinella, Azorhizobium, Paraburkholderia or Pseudomonas.

More specifically, the present invention further relates to the usage of a mutated chitin oligosaccharide synthase as described above wherein said transmembrane helices have an amino acid sequence identity of 30 to 100% (i.e. 30, 35, 40, 45, 50, 55, 60, 65, 70, 75, 80, 85, 90, 95 or 100%) to transmembrane helices of enzymes chosen from the group of: beta-polysaccharide synthase, chitin synthase, chitooligosaccharide synthase, N-acetylglucosaminyltransferase, beta-1,4-N-acetylglucosaminyltransferase, cellulose synthase, hyaluronan synthase, glycosyl transferase family 2, hyaluronic acid synthase, Nodulation protein C or NodC-like enzyme.

More specifically, the present invention further relates to the method to produce a mutated chitin oligosaccharide synthase as described above wherein said transmembrane helices have an amino acid sequence identity of 30 to 100% (i.e. 30, 35, 40, 45, 50, 55, 60, 65, 70, 75, 80, 85, 90, 95 or 100%) to transmembrane helices of enzymes chosen from the group of: beta-polysaccharide synthase, chitin synthase, chitooligosaccharide synthase, N-acetylglucosaminyltransferase, beta-1,4-N-acetylglucosaminyltransferase, cellulose synthase, hyaluronan synthase, glycosyl transferase family 2, hyaluronic acid synthase, Nodulation protein C or NodC-like enzyme.

More specifically, the present invention relates to the usage of a mutated, bacterial chitin oligosaccharide synthase as described above, wherein said transmembrane helix comprises the amino acid regions 187-200 and 295-370 of SEQ ID No 1, 190-203 and 298-373 of SEQ ID No 2, or, 190-200 and 298-373 of SEQ ID No 3, to increase at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

The present invention further relates to the usage of a mutated, bacterial chitin oligosaccharide synthase wherein said mutation is a substitution of at least one charged amino acid present in one of the two transmembrane helices of said chitin oligosaccharide synthase by an amino acid having the opposite charge or by an amino acid having no charge, wherein said substitution is not an arginine which is substituted by a serine at amino acid position 349 of the chitin oligosaccharide synthase consisting of the amino acid sequence given by SEQ ID No 2.

The present invention further relates to the method to produce a mutated, bacterial chitin oligosaccharide synthase wherein said mutation is a substitution of at least one charged amino acid present in one of the two transmembrane helices of said chitin oligosaccharide synthase by an amino acid having the opposite charge or by an amino acid having no charge, wherein said substitution is not an arginine which is substituted by a serine at amino acid position 349 of the chitin oligosaccharide synthase consisting of the amino acid sequence given by SEQ ID No 2.

More specifically, the present invention relates to a mutated, bacterial chitin oligosaccharide synthase wherein said mutation is a substitution of at least one charged amino acid present in one of the two transmembrane helices of said chitin oligosaccharide synthase by an amino acid having the opposite charge or by an amino acid having no charge wherein said transmembrane helix comprises the amino acid regions 187-200 and 295-370 of SEQ ID No 1, 190-203 and 298-373 of SEQ ID No 2 and 190-200 or 298-373 of SEQ ID No 3 in to any other opposite charged or neutral amino acid and, wherein said substitution is not an arginine which is substituted by a serine at amino acid position 349 of the chitin oligosaccharide synthase consisting of the amino acid sequence given by SEQ ID No 2.

Additionally, the present invention relates to a mutated, bacterial chitin oligosaccharide synthase wherein said mutation is a substitution of at least one charged amino acid present in one of the two transmembrane helices of said chitin oligosaccharide synthase by an amino acid having the opposite charge or by an amino acid having no charge, wherein said substitution is not an arginine which is substituted by a serine at amino acid position 349 of the chitin oligosaccharide synthase consisting of the amino acid sequence given by SEQ ID No 2.

Dorfmueller et. al (2014) suggested that increasing space in the catalytic cleft towards the formation of a transport channel through the membrane could increase the backbone length. They further suggested that to obtain such a channel, you should shorten the bulky side chains of amino acids (such as by substituting the bulky Arg at position 349 of SEQ ID No 2 by a shorter side chain amino acid) sterically blocking the growth of a COS transportation channel. However, the present invention discloses that introducing shorter side chain amino acids at position 349, i.e. Arg-349-Ser, in SmNodC yields an almost non-functional enzyme in vivo. Hence, an arginine which is substituted by a serine at amino acid position 349 of the chitin oligosaccharide synthase consisting of the amino acid sequence given by SEQ ID No 2 yielding SEQ ID No 24, (see further) shall not be an embodiment of the present invention!

Additionally, the present invention relates to a mutated, bacterial chitin oligosaccharide synthase as described above wherein said bacterial species belongs to the genus Rhizobium, Sinorhizobium, Cupriavidus, Burkholderia, Corallococcus, Desulfobacterium, Actinobacteria, Methylobacteria, Microvirga, Brucella, Bosea, Bradyrhizobium, Ochrobactrum, Devosia, Aminobacter, Mesorhizobium, Phyllobacterium, Agrobacterium, Allorhizobium, Neorhizobium, Shinella, Azorhizobium, Paraburkholderia or Pseudomonas

Additionally, the present invention further relates to a mutated chitin oligosaccharide synthase as described above wherein said transmembrane helices have an amino acid sequence identity of 30 to 100% (i.e. 30, 35, 40, 45, 50, 55, 60, 65, 70, 75, 80, 85, 90, 95 or 100%) to transmembrane helices of enzymes chosen from the group of: beta-polysaccharide synthase, chitin synthase, chitooligosaccharide synthase, N-acetylglucosaminyltransferase, beta-1,4-N-acetylglucosaminyltransferase, cellulose synthase, hyaluronan synthase, glycosyl transferase family 2, hyaluronic acid synthase, Nodulation protein C or NodC-like enzyme.

More specially, the present invention further relates to a mutated chitin oligosaccharide synthase as described above wherein said transmembrane helix comprises the amino acid regions 187-200 and 295-370 of SEQ ID No 1, 190-203 and 298-373 of SEQ ID No 2, or, 190-200 and 298-373 of SEQ ID No 3.

Additionally, said mutated enzyme with glycosyltransferase activity yields the formation of a chitin oligosaccharide with a degree of polymerization of four, five, six or seven with a purity in the total chitin oligosaccharide formation of 5%, 10%, 15%, 20%, 25%, 30%, 35%, 40%, 45%, 50%, 55%, 60%, 65%, 70%, 75%, 80%, 85%, 90%, 95% or 100%.

Additionally, said mutated enzyme with glycosyltransferase activity yields the formation of a chitin oligosaccharide with a degree of polymerization higher than three with a purity in the total chitin oligosaccharide formation of 5%, 10%, 15%, 20%, 25%, 30%, 35%, 40%, 45%, 50%, 55%, 60%, 65%, 70%, 75%, 80%, 85%, 90%, 95% or 100%.

Additionally, said mutated enzyme with glycosyltransferase activity yields the formation of a chitin oligosaccharide with a degree of polymerization higher than three and lower than eight with a purity in the total chitin oligosaccharide formation of 5%, 10%, 15%, 20%, 25%, 30%, 35%, 40%, 45%, 50%, 55%, 60%, 65%, 70%, 75%, 80%, 85%, 90%, 95% or 100%.

Additionally, said mutated enzyme with glycosyltransferase activity yields the formation of a chitin oligosaccharide with a degree of polymerization of four, five, six or seven with a purity in the total chitin oligosaccharide formation of 5%, 10%, 15%, 20%, 25%, 30%, 35%, 40%, 45%, 50%, 55%, 60%, 65%, 70%, 75%, 80%, 85%, 90%, 95% or 100%.

Additionally, said mutated enzyme with glycosyltransferase activity yields the formation of a chitin oligosaccharide with a degree of polymerization of six or seven with a purity in the total chitin oligosaccharide formation of 5%, 10%, 15%, 20%, 25%, 30%, 35%, 40%, 45%, 50%, 55%, 60%, 65%, 70%, 75%, 80%, 85%, 90%, 95% or 100%.

Additionally, said mutated enzyme with glycosyltransferase activity yields the formation of a chitin oligosaccharide with a degree of polymerization of four or five or six or seven with a purity of 100% Additionally, said mutated enzyme with glycosyltransferase activity yields the formation of a chitin oligosaccharide with a degree of polymerization of four or five or six with a purity of 100%.

More specifically, the present invention relates to a method wherein said enzyme with glycosyl transferase activity is mutated by substitution of at least one but not limited to one charged amino acid present in one of the two transmembrane helices of said enzyme with glycosyl transferase activity in to any other opposite charged or neutral amino acid.

More specifically, the present invention relates to a method wherein said enzyme with glycosyl transferase activity is mutated by substitution of at least one but not limited to one positively charged amino acid present in one of the two transmembrane helices of said enzyme with glycosyl transferase activity in to any other negatively charged or neutral amino acid.

More specifically, the present invention relates to a method wherein said enzyme with glycosyl transferase activity is mutated by substitution of at least one but not limited to one positively charged amino acid present along the transmembrane helix incorporating residues 187-200 and 295-370 of SEQ ID No 1, 190-203 and 298-373 of SEQ ID No 2 and 190-200 or 298-373 of SEQ ID No 3 in to any other opposite charged or neutral amino acid.

The present invention thus relates to a mutated chitin oligosaccharide synthase as described above wherein said charged amino acid is chosen from the list consisting of arginine, histidine and lysine, and, wherein said amino acid having the opposite charge is aspartic acid or glutamic acid.

More specifically, the present invention relates to a method wherein said enzyme with glycosyl transferase activity is mutated by substitution of at least one but not limited to one arginine or histidine present in one of the two transmembrane helices of said enzyme with glycosyl transferase activity in to any other negatively charged or neutral amino acid.

More specifically, the present invention relates to a method wherein said enzyme with glycosyl transferase activity is mutated by substitution of at least one but not limited to one arginine or histidine present along the transmembrane helix incorporating residues 187-200 and 295-370 of SEQ ID No 1, 190-203 and 298-373 of SEQ ID No 2 and 190-200 or 298-373 of SEQ ID No 3 in to any other opposite charged or neutral amino acid.

An example of the latter protein engineering strategy is a strategy wherein at least one arginine present along one of the two transmembrane helix of a N-acetylglucosamine transferase possibly (but not solely) originating from the bacterial genus Rhizobium, Sinorhizobium, Cupriavidus, Burkholderia, Corallococcus, Desulfobacterium, Actinobacteria, Methylobacteria, Microvirga, Brucella, Bosea, Bradyrhizobium, Ochrobactrum, Devosia, Aminobacter, Mesorhizobium, Phyllobacterium, Agrobacterium, Allorhizobium, Neorhizobium, Shinella, Azorhizobium, Paraburkholderia and Pseudomonas, having an amino acid sequence given by (but not solely) SEQ ID No 1-No 3, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, is substituted to a negatively charged amino acid yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

Another example of the latter protein engineering strategy is a strategy wherein at least one histidine present along one of the two transmembrane helix of a N-acetylglucosamine transferase possibly (but not solely) originating from the bacterial genus Rhizobium, Sinorhizobium, Cupriavidus, Burkholderia, Corallococcus, Desulfobacterium, Actinobacteria, Methylobacteria, Microvirga, Brucella, Bosea, Bradyrhizobium, Ochrobactrum, Devosia, Aminobacter, Mesorhizobium, Phyllobacterium, Agrobacterium, Allorhizobium, Neorhizobium, Shinella, Azorhizobium, Paraburkholderia and Pseudomonas, having an amino acid sequence given by (but not solely) SEQ ID No 1-No 3, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, is substituted to a negatively charged amino acid yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

The present invention thus further relates to a mutated chitin oligosaccharide synthase as described above wherein said charged amino acid is an arginine at position 192, 197, 346, 353, 355 or 358 of SEQ ID No 1.

The present invention thus further relates to a mutated chitin oligosaccharide synthase as described above wherein said charged amino acid is an arginine at position 197, 346, 353, 355 or 358 of SEQ ID No 1.

More specifically, the present invention relates to a mutated chitin oligosaccharide synthase as described above wherein said charged amino acid is an arginine at position 346 of SEQ ID No 1 and wherein an arginine at amino acid position 192, 197, 353, 355 or 358 of SEQ ID No 1 is substituted by glutamic acid or serine.

More specifically, the present invention relates to a mutated chitin oligosaccharide synthase as described above wherein said charged amino acid is an arginine at position 346 of SEQ ID No 1 and wherein an arginine at amino acid position 197, 353, 355 or 358 of SEQ ID No 1 is substituted by glutamic acid or serine.

The present invention further relates to a mutated chitin oligosaccharide synthase as described above wherein said charged amino acid is an arginine at position 346 of SEQ ID No 1 and wherein a methionine at amino acid position 342 of SEQ ID No 1 is substituted by serine, tryptophan or alanine.

The present invention further relates to a mutated chitin oligosaccharide synthase as described above wherein said charged amino acid is an arginine at position 346 of SEQ ID No 1 and wherein a histidine at amino acid position 298 of SEQ ID No 1 is substituted by threonine.

The present invention further relates to a mutated chitin oligosaccharide synthase as described above wherein said charged amino acid is a histidine at position 298 in SEQ ID No 1 and wherein said amino acid having the opposite charge is aspartic acid or glutamic acid.

In other words, the present invention relates to a protein engineering strategy wherein a N-acetylglucosamine transferase given by SEQ ID No 1, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides containing at least one of the following mutations R192D, R192E, R197D, R197E, H298D, H298E, R346D (SEQ ID No 4), R346E (SEQ ID No 5), R353D, R353E (SEQ ID No 6), R355D, R355E (SEQ ID No 7), R358D, R358E (SEQ ID No 8), H365D, H365E yielding chitin oligosaccharides an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention relates to a protein engineering strategy wherein a N-acetylglucosamine transferase given by SEQ ID No 1, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides containing at least one of the following mutations H298D, H298E, R346D (SEQ ID No 4), R346E (SEQ ID No 5), R353D, R353E (SEQ ID No 6), R355D, R355E (SEQ ID No 7), R358D, R358E (SEQ ID No 8), H365D, H365E yielding chitin oligosaccharides an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention relates to a protein engineering strategy wherein a N-acetylglucosamine transferase given by SEQ ID No 1, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides containing at least one of the following mutations R192D, R192E, R197D, R197E yielding chitin oligosaccharides an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

The present invention further relates to a protein engineering strategy wherein a N-acetylglucosamine transferase given by SEQ ID No 1, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides, containing at least one of the following mutations, R346D (SEQ ID No 4), R346E (SEQ ID No 5), R353E (SEQ ID No 6), R355E (SEQ ID No 7), R358E (SEQ ID No 8), yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention relates to a protein engineering strategy wherein a N-acetylglucosamine transferase given by SEQ ID No 1, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides containing at least one of the following double mutations R346D-R192E, R346D-R192S, R346E-R192E, R346E-R192S, R346D-R197E, R346D-R197S, R346E-R197E, R346E-R197S (SEQ ID No 12), R346D-R353E, R346D-R353S, R346E-R353E (SEQ ID No 9), R346E-R353S, R346D-R355E, R346D-R355S, R346E-R355E (SEQ ID No 10), R346E-R355S, R346D-R358E, R346D-R358S, R346E-R358E (SEQ ID No 11), R346E-R358S (SEQ ID No 13), R346D-M342S, R346E-M342S (SEQ ID No 15), R346D-M342W, R346E-M342W, R346D-M342A, R346E-M342A, R346D-H298T, R346E-H298T (SEQ ID No 14), yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention relates to a protein engineering strategy wherein a N-acetylglucosamine transferase given by SEQ ID No 1, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides containing at least one of the following mutations R346E-R197S (SEQ ID No 12), R346E-R353E (SEQ ID No 9), R346E-R355E (SEQ ID No 10), R346E-R358E (SEQ ID No 11), R346E-R358S (SEQ ID No 13), R346E-M342S (SEQ ID No 15), R346E-H298T (SEQ ID No 14), yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

The present invention further relates to a mutated chitin oligosaccharide synthase as described above wherein said charged amino acid is an arginine at position 195, 200, 349, 358 or 361 in SEQ ID No 2 and wherein said amino acid having the opposite charge is aspartic acid or glutamic acid.

The present invention further relates to a mutated chitin oligosaccharide synthase as described above wherein said charged amino acid is a histidine at position 327, 365 or 368 in SEQ ID No 2 and wherein said amino acid having the opposite charge is aspartic acid or glutamic acid.

The present invention further relates to a mutated chitin oligosaccharide synthase as described above wherein said charged amino acid is an arginine at position 200 or 349 in SEQ ID No 2 and wherein said amino acid having the opposite charge is aspartic acid or glutamic acid.

The present invention further relates to a mutated chitin oligosaccharide synthase as described above wherein said charged amino acid is an arginine at amino acid position 349 of SEQ ID No 2, wherein said amino acid having the opposite charge is aspartic acid or glutamic acid, and wherein an arginine at amino acid position 200 of SEQ ID No 2 is substituted by serine.

In other words, the present invention relates to a protein engineering strategy wherein a N-acetylglucosamine transferase given by SEQ ID No 2, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides, containing at least one of the following mutations R195D, R195E, R200D, R200E, H327D, H327E, R349D, R349E (SEQ ID No 25), H356D, H356E, R358D, R358E, R361D, R361E, H368D, H368E yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention relates to a protein engineering strategy wherein a N-acetylglucosamine transferase given by SEQ ID No 2, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides, containing at least one of the following mutations R195D, R195E, R200D, R200E yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention relates to a protein engineering strategy wherein a N-acetylglucosamine transferase given by SEQ ID No 2, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides, containing at least one of the following mutations H327D, H327E, R349D, R349E (SEQ ID No 25), H356D, H356E, R358D, R358E, R361D, R361E, H368D, H368E yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

The present invention further relates to a protein engineering strategy wherein a N-acetylglucosamine transferase given by SEQ ID No 2, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides, containing at least the following mutation R349E (SEQ ID No 25) yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention relates to a protein engineering strategy wherein a N-acetylglucosamine transferase given by SEQ ID No 2, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides, containing at least one of the following double mutations R349D-R200S, R349E-R200S (SEQ ID No 27) yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention relates to a protein engineering strategy wherein a N-acetylglucosamine transferase given by SEQ ID No 2, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides, containing at least one of the following double mutation R349E-R200S (SEQ ID No 27) yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

The present invention relates to a mutated chitin oligosaccharide synthase as described above wherein said charged amino acid is an arginine at position 195, 200, 301, 349, 358, 361 of SEQ ID No 3, and, wherein said amino acid having the opposite charge is aspartic acid or glutamic acid.

The present invention relates to a mutated chitin oligosaccharide synthase as described above wherein said charged amino acid is a histidine at position 356 or 368 of SEQ ID No 3, and, wherein said amino acid having the opposite charge is aspartic acid or glutamic acid.

The present invention relates to a mutated chitin oligosaccharide synthase as described above wherein said charged amino acid is an arginine at position 349 of SEQ ID No 3, and, wherein said amino acid having the opposite charge is aspartic acid or glutamic acid.

In other words, the present invention further relates to a protein engineering strategy wherein a N-acetylglucosamine transferase given by SEQ ID No 3, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides, containing at least one of the following mutations R195D, R195E, R200D, R200E, R301D, R301E, R349D, R349E (SEQ ID No 28), H356D, H356E, R358D, R358E, R361D, R361E, H368D, H368E yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention further relates to a protein engineering strategy wherein a N-acetylglucosamine transferase given by SEQ ID No 3, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides, containing at least one of the following mutations R195D, R195E, R200D, R200E yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention further relates to a protein engineering strategy wherein a N-acetylglucosamine transferase given by SEQ ID No 3, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides, containing at least one of the following mutations R301D, R301E, R349D, R349E (SEQ ID No 28), H356D, H356E, R358D, R358E, R361D, R361E, H368D, H368E yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention further relates to a protein engineering strategy wherein a N-acetylglucosamine transferase given by SEQ ID No 3, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides, containing at least the following mutation R349E (SEQ ID No 28) yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

The present invention relates to a mutated chitin oligosaccharide synthase as described above wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan.

The present invention relates to a mutated chitin oligosaccharide synthase as described above wherein said charged amino acid is chosen from the list consisting of an arginine, histidine and lysine and wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan.

The present invention relates to a mutated chitin oligosaccharide synthase as described above wherein said charged amino acid is an arginine or histidine within the amino acid regions 187-200 and 295-370 of SEQ ID No 1, 190-203 and 298-373 of SEQ ID No 2, or, 190-200 and 298-373 of SEQ ID No 3 and wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine or tryptophan.

The present invention relates to a mutated chitin oligosaccharide synthase as described above wherein said charged amino acid is an arginine at position 192, 197, 346, 353, 355 or 358 in SEQ ID No 1 and wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan.

The present invention relates to a mutated chitin oligosaccharide synthase as described above wherein said charged amino acid is an arginine at position 197, 346, 353, 355 or 358 in SEQ ID No 1 and wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan.

The present invention relates to a mutated chitin oligosaccharide synthase as described above wherein said charged amino acid is a histidine at position 298 in SEQ ID No 1 and wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan.

The present invention relates to a mutated chitin oligosaccharide synthase as described above wherein said charged amino acid is an arginine at amino acid position 346 of SEQ ID No 1, wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan, and wherein a methionine at amino acid position 342 of SEQ ID No 1 is substituted by serine, tryptophan, or alanine.

The present invention relates to a mutated chitin oligosaccharide synthase as described above wherein said charged amino acid is an arginine at amino acid position 346 of SEQ ID No 1, wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan and wherein a histidine at amino acid position 298 of SEQ ID No 1 is substituted by threonine.

The present invention relates to a mutated chitin oligosaccharide synthase as described above wherein said charged amino acid is an histidine at amino acid position 298 of SEQ ID No 1, wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan, and wherein a leucine at amino acid position 302 of SEQ ID No 1 is substituted by threonine.

In other words, the present invention further relates to a protein engineering strategy wherein at least one arginine or histidine present along one of the two transmembrane helix of a N-acetylglucosamine transferase possibly (but not solely) originating from the bacterial genus Rhizobium, Sinorhizobium, Cupriavidus, Burkholderia, Corallococcus, Desulfobacterium, Actinobacteria, Methylobacteria, Microvirga, Brucella, Bosea, Bradyrhizobium, Ochrobactrum, Devosia, Aminobacter, Mesorhizobium, Phyllobacterium, Agrobacterium, Allorhizobium, Neorhizobium, Shinella, Azorhizobium, Paraburkholderia and Pseudomonas, having an amino acid sequence given by (but not solely) SEQ ID No 1-No 3, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, is substituted to a neutral charged amino acid yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention further relates to a protein engineering strategy wherein a N-acetylglucosamine transferase given by SEQ ID No 1, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, contains at least one of the following mutations R192S, R192T, R192N, R192Q, R192C, R192G, R192P, R192A, R192V, R192I, R192L, R192M, R192F, R192Y, R192W, R197S (SEQ ID No 16), R197T, R197N, R197Q, R197C, R197G, R197P, R197A, R197V, R197I, R197L, R197M, R197F, R197Y, R197W, H298S, H298T, H298N, H298Q H298C, H298G, H298P, H298A, H298V, H298I, H298L, H298M, H298F, H298Y, H298W, R346S (SEQ ID No 17), R346T, R346N, R346Q (SEQ ID No 18), R346C, R346G, R346P, R346A, R346V, R346I, R346L, R346M, R346F, R346Y, R346W (SEQ ID No 19), R353S, R353T, R353N, R353Q, R353C, R353G, R353P, R353A, R353V, R353I, R353L, R353M, R353F, R353Y, R353W, R355S, R355T, R355N, R355Q, R355C, R355G, R355P, R355A, R355V, R355I, R355L, R355M, R355F, R355Y, R355W, R358S, R358T, R358N, R358Q, R358C, R358G, R358P, R358A, R358V, R358I, R358L, R358M, R358F, R358Y, R358W, H365S, H365T, H365N, H365Q, H365C, H365G, H365P, H365A, H365V, H365I, H365L, H365M, H365F, H365Y, H365W yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention further relates to a protein engineering strategy wherein a N-acetylglucosamine transferase given by SEQ ID No 1, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, contains at least one of the following mutations R192S, R192T, R192N, R192Q, R192C, R192G, R192P, R192A, R192V, R192I, R192L, R192M, R192F, R192Y, R192W, R197S (SEQ ID No 16), R197T, R197N, R197Q, R197C, R197G, R197P, R197A, R197V, R197I, R197L, R197M, R197F, R197Y, R197W, yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention further relates to a protein engineering strategy wherein a N-acetylglucosamine transferase given by SEQ ID No 1, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, contains at least one of the following mutations H298S, H298T, H298N, H298Q, H298C, H298G, H298P, H298A, H298V, H298I, H298L, H298M, H298F, H298Y, H298W, R346S (SEQ ID No 17), R346T, R346N, R346Q (SEQ ID No 18), R346C, R346G, R346P, R346A, R346V, R346I, R346L, R346M, R346F, R346Y, R346W (SEQ ID No 19), R353S, R353T, R353N, R353Q, R353C, R353G, R353P, R353A, R353V, R353I, R353L, R353M, R353F, R353Y, R353W, R355S, R355T, R355N, R355Q, R355C, R355G, R355P, R355A, R355V, R355I, R355L, R355M, R355F, R355Y, R355W, R358S, R358T, R358N, R358Q, R358C, R358G, R358P, R358A, R358V, R358I, R358L, R358M, R358F, R358Y, R358W, H365S, H365T, H365N, H365Q, H365C, H365G, H365P, H365A, H365V, H365I, H365L, H365M, H365F, H365Y, H365W yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

The present invention further relates to a protein engineering strategy wherein a N-acetylglucosamine transferase given by SEQ ID No 1, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, contains at least one of the following mutations R197S (SEQ ID No 16), R346S (SEQ ID No 17), R346Q (SEQ ID No 18), R346W (SEQ ID No 19) yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention further relates to a protein engineering strategy wherein a N-acetylglucosamine transferase given by SEQ ID No 1, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, contains at least one of the following double mutations H298S-L302T, H298T-L302T (SEQ ID No 23), H298N-L302T, H298Q-L302T, H298C-L302T, H298G-L302T, H298P-L302T, H298A-L302T, H298V-L302T, H298I-L302T, H298L-L302T, H298M-L302T, H298F-L302T, H298Y-L302T, H298W-L302T, R346S-M342S (SEQ ID No 21), R346T-M342S, R346N-M342S, R346Q-M342S, R346C-M342S, R346G-M342S, R346P-M342S, R346A-M342S, R346V-M342S, R346I-M342S, R346L-M342S, R346M-M342S, R346F-M342S, R346Y-M342S, R346W-M342S, R346S-M342W (SEQ ID No 22), R346T-M342W, R346N-M342W, R346Q-M342W, R346C-M342W, R346G-M342W, R346P-M342W, R346A-M342W, R346V-M342W, R346I-M342W, R346L-M342W, R346M-M342W, R346F-M342W, R346Y-M342W, R346W-M342W, R346S-M342A, R346T-M342A, R346N-M342A, R346Q-M342A, R346C-M342A, R346G-M342A, R346P-M342A, R346A-M342A, R346V-M342A, R346I-M342A, R346L-M342A, R346M-M342A, R346F-M342A, R346Y-M342A, R346W-M342A, R346S-H298T (SEQ ID No 20), R346T-H298T, R346N-H298T, R346Q-H298T, R346C-H298T, R346G-H298T, R346P-H298T, R346A-H298T, R346V-H298T, R346I-H298T, R346L-H298T, R346M-H298T, R346F-H298T, R346Y-H298T, R346W-H298T yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention further relates to a protein engineering strategy wherein a N-acetylglucosamine transferase given by SEQ ID No 1, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, contains at least one of the following double mutations H298T-L302T (SEQ ID No 23), R346S-M342S (SEQ ID No 21), R346S-M342W (SEQ ID No 22), R346S-H298T (SEQ ID No 20), yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

The present invention further relates to a mutated chitin oligosaccharide synthase wherein said charged amino acid is an arginine at position 200 or 349 of SEQ ID No 2 or at position 200 or 349 of SEQ ID No 3, and, wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan.

In other words, the present invention further relates to a protein engineering strategy wherein a N-acetylglucosamine transferase given by SEQ ID No 2, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, contains at least one of the following mutations R195S, R195T, R195N, R195Q, R195C, R195G, R195P, R195A, R195V, R195I, R195L, R195M, R195F, R195Y, R195W, R200S (SEQ ID No 26), R200T, R200N, R200Q, R200C, R200G, R200P, R200A, R200V, R200I, R200L, R200M, R200F, R200Y, R200W, H327S, H327T, H327N, H327Q H327C, H327G, H327P, H327A, H327V, H327I, H327L, H327M, H327F, H327Y, H327W, R349T, R349N, R349Q R349C, R349G, R349P, R349A, R349V, R349I, R349L, R349M, R349F, R349Y, R349W, H356S, H356T, H356N, H356Q, H356C, H356G, H356P, H356A, H356V, H356I, H356L, H356M, H356F, H356Y, H356W, R358S, R358T, R358N, R358Q, R358C, R358G, R358P, R358A, R358V, R358I, R358L, R358M, R358F, R358Y, R358W, R361S, R361T, R361N, R361Q, R361C, R361G, R361P, R361A, R361V, R361I, R361L, R361M, R361F, R361Y, R361W, H368S, H368T, H368N, H368Q, H368C, H368G, H368P, H368A, H368V, H368I, H368L, H368M, H368F, H368Y, H368W yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention further relates to a protein engineering strategy wherein a N-acetylglucosamine transferase given by SEQ ID No 2, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, contains at least one of the following mutations R195S, R195T, R195N, R195Q, R195C, R195G, R195P, R195A, R195V, R195I, R195L, R195M, R195F, R195Y, R195W, R200S (SEQ ID No 26), R200T, R200N, R200Q, R200C, R200G, R200P, R200A, R200V, R200I, R200L, R200M, R200F, R200Y, R200W yielding chitin oligosaccharides an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention further relates to a protein engineering strategy wherein a N-acetylglucosamine transferase given by SEQ ID No 2, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, contains at least one of the following mutations H327S, H327T, H327N, H327Q H327C, H327G, H327P, H327A, H327V, H327I, H327L, H327M, H327F, H327Y, H327W, R349T, R349N, R349Q R349C, R349G, R349P, R349A, R349V, R349I, R349L, R349M, R349F, R349Y, R349W, H356S, H356T, H356N, H356Q H356C, H356G, H356P, H356A, H356V, H356I, H356L, H356M, H356F, H356Y, H356W, R358S, R358T, R358N, R358Q R358C, R358G, R358P, R358A, R358V, R358I, R358L, R358M, R358F, R358Y, R358W, R361S, R361T, R361N, R361Q, R361C, R361G, R361P, R361A, R361V, R361I, R361L, R361M, R361F, R361Y, R361W, H368S, H368T, H368N, H368Q, H368C, H368G, H368P, H368A, H368V, H368I, H368L, H368M, H368F, H368Y, H368W yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention further relates to a protein engineering strategy wherein a N-acetylglucosamine transferase given by SEQ ID No 2, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, contains at least one of the following mutations R200S (SEQ ID No 26), yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention further relates to a protein engineering strategy wherein a N-acetylglucosamine transferase given by SEQ ID No 3, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, containing at least one of the following mutations R195S, R195T, R195N, R195Q, R195C, R195G, R195P, R195A, R195V, R195I, R195L, R195M, R195F, R195Y, R195W, R200S, R200T, R200N, R200Q, R200C, R200G, R200P, R200A, R200V, R200I, R200L, R200M, R200F, R200Y, R200W, R301S, R301T, R301N, R301Q, R301C, R301G, R301P, R301A, R301V, R301I, R301L, R301M, R301F, R301Y, R301W, R349S (SEQ ID No 29), R349T, R349N, R349Q, R349C, R349G, R349P, R349A, R349V, R349I, R349L, R349M, R349F, R349Y, R349W, H356S, H356T, H356N, H356Q H356C, H356G, H356P, H356A, H356V, H356I, H356L, H356M, H356F, H356Y, H356W, R358S, R358T, R358N, R358Q R358C, R358G, R358P, R358A, R358V, R358I, R358L, R358M, R358F, R358Y, R358W, R361S, R361T, R361N, R361Q, R361C, R361G, R361P, R361A, R361V, R361I, R361L, R361M, R361F, R361Y, R361W, H368S, H368T, H368N, H368Q, H368C, H368G, H368P, H368A, H368V, H368I, H368L, H368M, H368F, H368Y, H368W yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention further relates to a protein engineering strategy wherein a N-acetylglucosamine transferase given by SEQ ID No 3, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, containing at least one of the following mutations R195S, R195T, R195N, R195Q, R195C, R195G, R195P, R195A, R195V, R195I, R195L, R195M, R195F, R195Y, R195W, R200S, R200T, R200N, R200Q, R200C, R200G, R200P, R200A, R200V, R200I, R200L, R200M, R200F, R200Y, R200W yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention further relates to a protein engineering strategy wherein a N-acetylglucosamine transferase given by SEQ ID No 3, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, containing at least one of the following mutations R301S, R301T, R301N, R301Q, R301C, R301G, R301P, R301A, R301V, R301I, R301L, R301M, R301F, R301Y, R301W, R349S (SEQ ID No 29), R349T, R349N, R349Q, R349C, R349G, R349P, R349A, R349V, R349I, R349L, R349M, R349F, R349Y, R349W, H356S, H356T, H356N, H356Q, H356C, H356G, H356P, H356A, H356V, H356I, H356L, H356M, H356F, H356Y, H356W, R358S, R358T, R358N, R358Q, R358C, R358G, R358P, R358A, R358V, R358I, R358L, R358M, R358F, R358Y, R358W, R361S, R361T, R361N, R361Q, R361C, R361G, R361P, R361A, R361V, R361I, R361L, R361M, R361F, R361Y, R361W, H368S, H368T, H368N, H368Q, H368C, H368G, H368P, H368A, H368V, H368I, H368L, H368M, H368F, H368Y, H368W yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

In other words, the present invention further relates to a protein engineering strategy wherein a N-acetylglucosamine transferase given by SEQ ID No 3, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) and having a chitin oligosaccharide synthase activity to produce chitin oligosaccharides, containing at least one of the following mutations R349S (SEQ ID No 29) yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

The present invention further relates to said mutated enzyme with glycosyltransferase activity as indicated above wherein the amino acid sequence of said mutated enzyme is further engineered by combining the above mentioned engineering strategies.

More specifically, the present invention relates to an above described mutated enzyme with glycosyl transferase activity having a substitution of one charged amino acid present in one of the two transmembrane helices of said enzyme with glycosyl transferase activity in to any other opposite charged or neutral amino acid, contains an additional modification by substitution of at least one but not limited to one charged amino acid present in one of the two transmembrane helices of said enzyme with glycosyl transferase activity in to any other opposite charged or neutral amino acid.

More specifically, the present invention relates to an above described mutated enzyme with glycosyl transferase activity having a substitution of one charged amino acid present along the transmembrane helix incorporating residues 187-200 and 295-370 of SEQ ID No 1, 190-203 and 298-373 of SEQ ID No 2 and 190-200 or 298-373 of SEQ ID No 3 in to any other opposite charged or neutral amino acid, contains an additional modification by substitution of at least one but not limited to one charged amino acid present along the transmembrane helix incorporating residues 187-200 and 295-370 of SEQ ID No 1, 190-203 and 298-373 of SEQ ID No 2 and 190-200 or 298-373 of SEQ ID No 3 in to any other opposite charged or neutral amino acid.

More specifically, the present invention relates to an above described mutated enzyme with glycosyl transferase activity having a substitution of one positively charged amino acid present in one of the two transmembrane helices of said enzyme with glycosyl transferase activity in to any other opposite charged or neutral amino acid, contains an additional modification by substitution of at least one but not limited to one charged amino acid present in one of the two transmembrane helices of said enzyme with glycosyl transferase activity in to any other opposite charged or neutral amino acid.

More specifically, the present invention relates to an above described mutated enzyme with glycosyl transferase activity having a substitution of one positively charged amino acid present along the transmembrane helix incorporating residues 187-200 and 295-370 of SEQ ID No 1, 190-203 and 298-373 of SEQ ID No 2 and 190-200 or 298-373 of SEQ ID No 3 in to any other opposite charged or neutral amino acid, contains an additional modification by substitution of at least one but not limited to one charged amino acid present along the transmembrane helix incorporating residues 187-200 and 295-370 of SEQ ID No 1, 190-203 and 298-373 of SEQ ID No 2 and 190-200 or 298-373 of SEQ ID No 3 in to any other opposite charged or neutral amino acid.

More specifically, the present invention relates to an above described mutated enzyme with glycosyl transferase activity having a substitution of an arginine or histidine present in one of the two transmembrane helices of said enzyme with glycosyl transferase activity in to any other opposite charged or neutral amino acid, contains an additional modification by substitution of at least one but not limited to one charged amino acid present in one of the two transmembrane helices of said enzyme with glycosyl transferase activity in to any other opposite charged or neutral amino acid.

More specifically, the present invention relates to an above described mutated enzyme with glycosyl transferase activity having a substitution of an arginine or histidine present along the transmembrane helix incorporating residues 187-200 and 295-370 of SEQ ID No 1, 190-203 and 298-373 of SEQ ID No 2 and 190-200 or 298-373 of SEQ ID No 3 in to any other opposite charged or neutral amino acid, contains an additional modification by substitution of at least one but not limited to one charged amino acid present along the transmembrane helix incorporating residues 187-200 and 295-370 of SEQ ID No 1, 190-203 and 298-373 of SEQ ID No 2 and 190-200 or 298-373 of SEQ ID No 3 in to any other opposite charged or neutral amino acid.

An example of the above described protein engineering strategy is a strategy wherein additionally to one arginine or histidine present along one of the two transmembrane helix of a N-acetylglucosamine transferase possibly (but not solely) originating from the bacterial genus Rhizobium, Sinorhizobium, Cupriavidus, Burkholderia, Corallococcus, Desulfobacterium, Actinobacteria, Methylobacteria, Microvirga, Brucella, Bosea, Bradyrhizobium, Ochrobactrum, Devosia, Aminobacter, Mesorhizobium, Phyllobacterium, Agrobacterium, Allorhizobium, Neorhizobium, Shinella, Azorhizobium, Paraburkholderia and Pseudomonas, having an amino acid sequence given by (but not solely) SEQ ID No 1-No 3, or, a fragment thereof having a chitin oligosaccharide synthase activity, or, a variant thereof having a sequence identity of at least 75% and having a chitin oligosaccharide synthase activity to produce chitin oligosaccharides, being substituted to a negatively charged amino acid, at least one but not limited to one charged amino acid present along the transmembrane helix is substituted by a negatively charged or neutral amino acid yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

Another example of the the above described protein engineering strategy is a strategy wherein a N-acetylglucosamine transferase given by SEQ ID No 1, or, a fragment thereof having a chitin oligosaccharide synthase activity, or, a variant thereof having a sequence identity of at least 75% and having a chitin oligosaccharide synthase activity to produce chitin oligosaccharides, contains at least one of the following mutations R192D, R192E, R197D, R197E, H298D, H298E, R346D, R346E, R353D, R353E, R355D, R355E, R358D, R358E, H365D, H365E combined with at least one of the following mutations R192D, R192E, R197D, R197E, H298D, H298E, R346D, R346E, R353D, R353E, R355D, R355E, R358D, R358E, H365D, H365E, R192S, R192T, R192N, R192Q, R192C, R192G, R192P, R192A, R192V, R192I, R192L, R192M, R192F, R192Y, R192W, R197S, R197T, R197N, R197Q, R197C, R197G, R197P, R197A, R197V, R197I, R197L, R197M, R197F, R197Y, R197W, H298S, H298T, H298N, H298Q, H298C, H298G, H298P, H298A, H298V, H298I, H298L, H298M, H298F, H298Y, H298W, R346S, R346T, R346N, R346Q, R346C, R346G, R346P, R346A, R346V, R346I, R346L, R346M, R346F, R346Y, R346W, R353S, R353T, R353N, R353Q, R353C, R353G, R353P, R353A, R353V, R353I, R353L, R353M, R353F, R353Y, R353W, R355S, R355T, R355N, R355Q, R355C, R355G, R355P, R355A, R355V, R355I, R355L, R355M, R355F, R355Y, R355W, R358S, R358T, R358N, R358Q, R358C, R358G, R358P, R358A, R358V, R358I, R358L, R358M, R358F, R358Y, R358W, H365S, H365T, H365N, H365Q, H365C, H365G, H365P, H365A, H365V, H365I, H365L, H365M, H365F, H365Y, H365W yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

Another example of the the above described protein engineering strategy is a strategy wherein a N-acetylglucosamine transferase given by SEQ ID No 1, or, a fragment thereof having a chitin oligosaccharide synthase activity, or, a variant thereof having a sequence identity of at least 75% (i.e. 75, 76, 77, 78, 79, 80, 81, 82, 83, 84, 85, 86, 87, 88, 89, 90, 91, 92, 93, 94, 95, 96, 97, 98 or 99%) have a chitin oligosaccharide synthase activity to produce chitin oligosaccharides containing at least one of the following mutations R346E-R197S (SEQ ID No 12), R346E-R353E (SEQ ID No 9), R346E-R355E (SEQ ID No 10), R346E-R358E (SEQ ID No 11), R346E-R358S (SEQ ID No 13), R346E-M342S (SEQ ID No 15), R346E-H298T (SEQ ID No 14), yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

Another example of the above described protein engineering strategy is a strategy wherein a N-acetylglucosamine transferase given by SEQ ID No 2, or, a fragment thereof having a chitin oligosaccharide synthase activity, or, a variant thereof having a sequence identity of at least 75% and having a chitin oligosaccharide synthase activity to produce chitin oligosaccharides, contains at least one of the following mutations R195D, R195E, R200D, R200E, H327D, H327E, R349D, R349E, H356D, H356E, R358D, R358E, R361D, R361E, H368D, H368E combined with at least one of the following mutations R195D, R195E, R200D, R200E, H327D, H327E, R349D, R349E, H356D, H356E, R358D, R358E, R361D, R361E, H368D, H368E, R195S, R195T, R195N, R195Q, R195C, R195G, R195P, R195A, R195V, R195I, R195L, R195M, R195F, R195Y, R195W, R200S, R200T, R200N, R200Q, R200C, R200G, R200P, R200A, R200V, R200I, R200L, R200M, R200F, R200Y, R200W, H327S, H327T, H327N, H327Q, H327C, H327G, H327P, H327A, H327V, H327I, H327L, H327M, H327F, H327Y, H327W, R349T, R349N, R349Q, R349C, R349G, R349P, R349A, R349V, R349I, R349L, R349M, R349F, R349Y, R349W, H356S, H356T, H356N, H356Q, H356C, H356G, H356P, H356A, H356V, H356I, H356L, H356M, H356F, H356Y, H356W, R358S, R358T, R358N, R358Q, R358C, R358G, R358P, R358A, R358V, R358I, R358L, R358M, R358F, R358Y, R358W, R361S, R361T, R361N, R361Q, R361C, R361G, R361P, R361A, R361V, R361I, R361L, R361M, R361F, R361Y, R361W, H368S, H368T, H368N, H368Q, H368C, H368G, H368P, H368A, H368V, H368I, H368L, H368M, H368F, H368Y, H368W yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

Another example of the above described protein engineering strategy is a strategy wherein a N-acetylglucosamine transferase given by SEQ ID No 2, or, a fragment thereof having a chitin oligosaccharide synthase activity, or, a variant thereof having a sequence identity of at least 75% and having a chitin oligosaccharide synthase activity to produce chitin oligosaccharides, contains at least the following mutation R349E-R200S (SEQ ID No 27) yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

An example of the above described protein engineering strategy is a strategy wherein additionally to one arginine or histidine present along one of the two transmembrane helix of a N-acetylglucosamine transferase possibly (but not solely) originating from the bacterial genus Rhizobium, Sinorhizobium, Cupriavidus, Burkholderia, Corallococcus, Desulfobacterium, Actinobacteria, Methylobacteria, Microvirga, Brucella, Bosea, Bradyrhizobium, Ochrobactrum, Devosia, Aminobacter, Mesorhizobium, Phyllobacterium, Agrobacterium, Allorhizobium, Neorhizobium, Shinella, Azorhizobium, Paraburkholderia and Pseudomonas, having an amino acid sequence given by (but not solely) SEQ ID No 1-No 3, or, a fragment thereof having a chitin oligosaccharide synthase activity, or, a variant thereof having a sequence identity of at least 75% and having a chitin oligosaccharide synthase activity to produce chitin oligosaccharides, being substituted to a neutral amino acid, at least one but not limited to one charged amino acid present along the transmembrane helix is substituted by a negatively charged or neutral amino acid yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

Another example of the above described protein engineering strategy is a strategy wherein a N-acetylglucosamine transferase given by SEQ ID No 1, or, a fragment thereof having a chitin oligosaccharide synthase activity, or, a variant thereof having a sequence identity of at least 75% and having a chitin oligosaccharide synthase activity to produce chitin oligosaccharides, contains at least one of the following mutations R192S, R192T, R192N, R192Q, R192C, R192G, R192P, R192A, R192V, R192I, R192L, R192M, R192F, R192Y, R192W, R197S, R197T, R197N, R197Q, R197C, R197G, R197P, R197A, R197V, R197I, R197L, R197M, R197F, R197Y, R197W, H298S, H298T, H298N, H298Q, H298C, H298G, H298P, H298A, H298V, H298I, H298L, H298M, H298F, H298Y, H298W, R346S, R346T, R346N, R346Q, R346C, R346G, R346P, R346A, R346V, R346I, R346L, R346M, R346F, R346Y, R346W, R353S, R353T, R353N, R353Q, R353C, R353G, R353P, R353A, R353V, R353I, R353L, R353M, R353F, R353Y, R353W, R355S, R355T, R355N, R355Q, R355C, R355G, R355P, R355A, R355V, R355I, R355L, R355M, R355F, R355Y, R355W, R358S, R358T, R358N, R358Q R358C, R358G, R358P, R358A, R358V, R358I, R358L, R358M, R358F, R358Y, R358W, H365S, H365T, H365N, H365Q H365C, H365G, H365P, H365A, H365V, H365I, H365L, H365M, H365F, H365Y, H365W combined with at least one of the following mutations R192D, R192E, R197D, R197E, H298D, H298E, R346D, R346E, R353D, R353E, R355D, R355E, R358D, R358E, H365D, H365E, R192S, R192T, R192N, R192Q, R192C, R192G, R192P, R192A, R192V, R192I, R192L, R192M, R192F, R192Y, R192W, R197S, R197T, R197N, R197Q, R197C, R197G, R197P, R197A, R197V, R197I, R197L, R197M, R197F, R197Y, R197W, H298S, H298T, H298N, H298Q, H298C, H298G, H298P, H298A, H298V, H298I, H298L, H298M, H298F, H298Y, H298W, R346S, R346T, R346N, R346Q, R346C, R346G, R346P, R346A, R346V, R346I, R346L, R346M, R346F, R346Y, R346W, R353S, R353T, R353N, R353Q, R353C, R353G, R353P, R353A, R353V, R353I, R353L, R353M, R353F, R353Y, R353W, R355S, R355T, R355N, R355Q, R355C, R355G, R355P, R355A, R355V, R355I, R355L, R355M, R355F, R355Y, R355W, R358S, R358T, R358N, R358Q R358C, R358G, R358P, R358A, R358V, R358I, R358L, R358M, R358F, R358Y, R358W, H365S, H365T, H365N, H365Q H365C, H365G, H365P, H365A, H365V, H365I, H365L, H365M, H365F, H365Y, H365W yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

Another example of the above described protein engineering strategy is a strategy wherein a N-acetylglucosamine transferase given by SEQ ID No 1, or, a fragment thereof having a chitin oligosaccharide synthase activity, or, a variant thereof having a sequence identity of at least 75% and having a chitin oligosaccharide synthase activity to produce chitin oligosaccharides, contains at least one of the following mutations R346E-R197S (SEQ ID No 12), R346E-H298T (SEQ ID No 14), R346S-H298T (SEQ ID No 20) yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

Another example of the above described protein engineering strategy is a strategy wherein a N-acetylglucosamine transferase given by SEQ ID No 2, or, a fragment thereof having a chitin oligosaccharide synthase activity, or, a variant thereof having a sequence identity of at least 75% and having a chitin oligosaccharide synthase activity to produce chitin oligosaccharides, contains at least one of the following mutations R195S, R195T, R195N, R195Q, R195C, R195G, R195P, R195A, R195V, R195I, R195L, R195M, R195F, R195Y, R195W, R200S, R200T, R200N, R200Q, R200C, R200G, R200P, R200A, R200V, R200I, R200L, R200M, R200F, R200Y, R200W, H327S, H327T, H327N, H327Q H327C, H327G, H327P, H327A, H327V, H327I, H327L, H327M, H327F, H327Y, H327W, R349T, R349N, R349Q R349C, R349G, R349P, R349A, R349V, R349I, R349L, R349M, R349F, R349Y, R349W, H356S, H356T, H356N, H356Q H356C, H356G, H356P, H356A, H356V, H356I, H356L, H356M, H356F, H356Y, H356W, R358S, R358T, R358N, R358Q R358C, R358G, R358P, R358A, R358V, R358I, R358L, R358M, R358F, R358Y, R358W, R361S, R361T, R361N, R361Q, R361C, R361G, R361P, R361A, R361V, R361I, R361L, R361M, R361F, R361Y, R361W, H368S, H368T, H368N, H368Q H368C, H368G, H368P, H368A, H368V, H368I, H368L, H368M, H368F, H368Y, H368W combined with at least one of the following mutations R195D, R195E, R200D, R200E, H327D, H327E, R349D, R349E, H356D, H356E, R358D, R358E, R361D, R361E, H368D, H368E, R195S, R195T, R195N, R195Q, R195C, R195G, R195P, R195A, R195V, R195I, R195L, R195M, R195F, R195Y, R195W, R200S, R200T, R200N, R200Q, R200C, R200G, R200P, R200A, R200V, R200I, R200L, R200M, R200F, R200Y, R200W, H327S, H327T, H327N, H327Q, H327C, H327G, H327P, H327A, H327V, H327I, H327L, H327M, H327F, H327Y, H327W, R349T, R349N, R349Q R349C, R349G, R349P, R349A, R349V, R349I, R349L, R349M, R349F, R349Y, R349W, H356S, H356T, H356N, H356Q H356C, H356G, H356P, H356A, H356V, H356I, H356L, H356M, H356F, H356Y, H356W, R358S, R358T, R358N, R358Q R358C, R358G, R358P, R358A, R358V, R358I, R358L, R358M, R358F, R358Y, R358W, R361S, R361T, R361N, R361Q, R361C, R361G, R361P, R361A, R361V, R361I, R361L, R361M, R361F, R361Y, R361W, H368S, H368T, H368N, H368Q, H368C, H368G, H368P, H368A, H368V, H368I, H368L, H368M, H368F, H368Y, H368W yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

More specifically, the present invention relates to a method wherein said mutated enzyme with glycosyl transferase activity having a substitution of one charged amino acid present in one of the two transmembrane helices of said enzyme with glycosyl transferase activity in to any other opposite charged or neutral amino acid, contains an additional modification by substitution of a methionine in to serine, alanine or tryptophan or by substitution of a leucine in to threonine in one of the two transmembrane helices of said enzyme with glycosyl transferase activity.

More specifically, the present invention relates to a method wherein said mutated enzyme with glycosyl transferase activity having a substitution of one charged amino acid present along the transmembrane helix incorporating residues 187-200 and 295-370 of SEQ ID No 1, 190-203 and 298-373 of SEQ ID No 2 and 190-200 or 298-373 of SEQ ID No 3 in to any other opposite charged or neutral amino acid, contains an additional modification by substitution of a methionine present along the transmembrane helix incorporating residues 187-200 and 295-370 of SEQ ID No 1, 190-203 and 298-373 of SEQ ID No 2 and 190-200 or 298-373 of SEQ ID No 3 in to serine, alanine or tryptophan or by substitution of a leucine present along the transmembrane helix incorporating residues 187-200 and 295-370 of SEQ ID No 1, 190-203 and 298-373 of SEQ ID No 2 and 190-200 or 298-373 of SEQ ID No 3 in to threonine.

More specifically, the present invention relates to a method wherein said mutated enzyme with glycosyl transferase activity having a substitution of one positively charged amino acid present in one of the two transmembrane helices of said enzyme with glycosyl transferase activity in to any other negatively charged or neutral amino acid, contains an additional modification by substitution of a methionine in to serine, alanine or tryptophan or by substitution of a leucine in to threonine in one of the two transmembrane helices of said enzyme with glycosyl transferase activity.

More specifically, the present invention relates to a method wherein said mutated enzyme with glycosyl transferase activity having a substitution of one positively charged amino acid present along the transmembrane helix incorporating residues 187-200 and 295-370 of SEQ ID No 1, 190-203 and 298-373 of SEQ ID No 2 and 190-200 or 298-373 of SEQ ID No 3 in to any other negatively charged or neutral amino acid, contains an additional modification by substitution of a methionine present along the transmembrane helix incorporating residues 187-200 and 295-370 of SEQ ID No 1, 190-203 and 298-373 of SEQ ID No 2 and 190-200 or 298-373 of SEQ ID No 3 in to serine, alanine or tryptophan or by substitution of a leucine present along the transmembrane helix incorporating residues 187-200 and 295-370 of SEQ ID No 1, 190-203 and 298-373 of SEQ ID No 2 and 190-200 or 298-373 of SEQ ID No 3 in to threonine.

More specifically, the present invention relates to a method wherein said mutated enzyme with glycosyl transferase activity having a substitution of one arginine or histidine present in one of the two transmembrane helices of said enzyme with glycosyl transferase activity in to any other negatively charged or neutral amino acid, contains an additional modification by substitution of a methionine in to serine, alanine or tryptophan or by substitution of a leucine in to threonine in one of the two transmembrane helices of said enzyme with glycosyl transferase activity.

More specifically, the present invention relates to a method wherein said mutated enzyme with glycosyl transferase activity having a substitution of one arginine or histidine present along the transmembrane helix incorporating residues 187-200 and 295-370 of SEQ ID No 1, 190-203 and 298-373 of SEQ ID No 2 and 190-200 or 298-373 of SEQ ID No 3 in to any other negatively charged or neutral amino acid, contains an additional modification by substitution of a methionine present along the transmembrane helix incorporating residues 187-200 and 295-370 of SEQ ID No 1, 190-203 and 298-373 of SEQ ID No 2 and 190-200 or 298-373 of SEQ ID No 3 in to serine, alanine or tryptophan or by substitution of a leucine present along the transmembrane helix incorporating residues 187-200 and 295-370 of SEQ ID No 1, 190-203 and 298-373 of SEQ ID No 2 and 190-200 or 298-373 of SEQ ID No 3 in to threonine.

An example of the above described protein engineering strategy is a strategy wherein additionally to one arginine or histidine present along one of the two transmembrane helix of a N-acetylglucosamine transferase possibly (but not solely) originating from the bacterial genus Rhizobium, Sinorhizobium, Cupriavidus, Burkholderia, Corallococcus, Desulfobacterium, Actinobacteria, Methylobacteria, Microvirga, Brucella, Bosea, Bradyrhizobium, Ochrobactrum, Devosia, Aminobacter, Mesorhizobium, Phyllobacterium, Agrobacterium, Allorhizobium, Neorhizobium, Shinella, Azorhizobium, Paraburkholderia and Pseudomonas, having an amino acid sequence given by (but not solely) SEQ ID No 1-No 3, or, a fragment thereof having a chitin oligosaccharide synthase activity, or, a variant thereof having a sequence identity of at least 75% and having a chitin oligosaccharide synthase activity to produce chitin oligosaccharides, being substituted to a neutral amino acid, contains a substitution of a methionine in to serine, alanine or tryptophan or a substitution of a leucine in to threonine in one of the two transmembrane helices of said enzyme with glycosyl transferase activity yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

Another example of the above-described protein engineering strategy is a strategy wherein a N-acetylglucosamine transferase given by SEQ ID No 1, or, a fragment thereof having a chitin oligosaccharide synthase activity, or, a variant thereof having a sequence identity of at least 75% and having a chitin oligosaccharide synthase activity to produce chitin oligosaccharides, contains at least one of the following mutations R192D, R192E, R197D, R197E, H298D, H298E, R346D, R346E, R353D, R353E, R355D, R355E, R358D, R358E, H365D, H365E, R192S, R192T, R192N, R192Q, R192C, R192G, R192P, R192A, R192V, R192I, R192L, R192M, R192F, R192Y, R192W, R197S, R197T, R197N, R197Q, R197C, R197G, R197P, R197A, R197V, R197I, R197L, R197M, R197F, R197Y, R197W, H298S, H298T, H298N, H298Q, H298C, H298G, H298P, H298A, H298V, H298I, H298L, H298M, H298F, H298Y, H298W, R346S, R346T, R346N, R346Q, R346C, R346G, R346P, R346A, R346V, R346I, R346L, R346M, R346F, R346Y, R346W, R353S, R353T, R353N, R353Q, R353C, R353G, R353P, R353A, R353V, R353I, R353L, R353M, R353F, R353Y, R353W, R355S, R355T, R355N, R355Q, R355C, R355G, R355P, R355A, R355V, R355I, R355L, R355M, R355F, R355Y, R355W, R358S, R358T, R358N, R358Q R358C, R358G, R358P, R358A, R358V, R358I, R358L, R358M, R358F, R358Y, R358W, H365S, H365T, H365N, H365Q, H365C, H365G, H365P, H365A, H365V, H365I, H365L, H365M, H365F, H365Y, H365W combined with at least one of the following mutations M342S, M342A, M342W, L302T yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

Another example of the above-described protein engineering strategy is a strategy wherein a N-acetylglucosamine transferase given by SEQ ID No 1, or, a fragment thereof having a chitin oligosaccharide synthase activity, or, a variant thereof having a sequence identity of at least 75% and having a chitin oligosaccharide synthase activity to produce chitin oligosaccharides, contains at least one of the following mutations R346S-M342S (SEQ ID No 21), R346S-M342A, R346S-M342W (SEQ ID No 22), H298T-L302T (SEQ ID No 23) yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

Another example of the the above described protein engineering strategy is a strategy wherein a N-acetylglucosamine transferase given by SEQ ID No 2, or, a fragment thereof having a chitin oligosaccharide synthase activity, or, a variant thereof having a sequence identity of at least 75% and having a chitin oligosaccharide synthase activity to produce chitin oligosaccharides, contains at least one of the following mutations R195D, R195E, R200D, R200E, H327D, H327E, R349D, R349E, H356D, H356E, R358D, R358E, R361D, R361E, H368D, H368E, R195S, R195T, R195N, R195Q, R195C, R195G, R195P, R195A, R195V, R195I, R195L, R195M, R195F, R195Y, R195W, R200S, R200T, R200N, R200Q, R200C, R200G, R200P, R200A, R200V, R200I, R200L, R200M, R200F, R200Y, R200W, H327S, H327T, H327N, H327Q, H327C, H327G, H327P, H327A, H327V, H327I, H327L, H327M, H327F, H327Y, H327W, R349T, R349N, R349Q, R349C, R349G, R349P, R349A, R349V, R349I, R349L, R349M, R349F, R349Y, R349W, H356S, H356T, H356N, H356Q, H356C, H356G, H356P, H356A, H356V, H356I, H356L, H356M, H356F, H356Y, H356W, R358S, R358T, R358N, R358Q R358C, R358G, R358P, R358A, R358V, R358I, R358L, R358M, R358F, R358Y, R358W, R361S, R361T, R361N, R361Q, R361C, R361G, R361P, R361A, R361V, R361I, R361L, R361M, R361F, R361Y, R361W, H368S, H368T, H368N, H368Q, H368C, H368G, H368P, H368A, H368V, H368I, H368L, H368M, H368F, H368Y, H368W combined with at least one of the following mutations M342S, M342A, M342W, L302T yielding chitin oligosaccharides with an increase in at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.

The following specific sequences, as indicated above, are:

TABLE 1
Sequences of used enzymes

| SEQ ID No | Enzyme | Protein sequence (Genbank accession code if existing) |
|---|---|---|
| 1 | RsNodC | AJW76243 |
| | | mdlIntigig avscyallst ahksmqtlya qpkdqssase dfaflpsvdi ivpcynenph |
| | | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia smtmircava |
| | | avrarqlrfl vfslhtpinl ffllpmkaya Ictlsnsdwl srsspankts aggehpttea |
| | | saggtsgnat plrrInlard sstvtpagvy sdd |
| 2 | SmNodC | AAK65131.1 |
| | | mylldttsta aisiyalllt ayrsmqvlha rpidgpavsa epvetrplpa vdvivpsfne |
| | | dpgilsacla siadqdypge Irvyvvddgs rnreailrvr afysrdprfs fillpenvgk |
| | | rkaqiaaigq ssgdlvlnvd sdstiafdvv sklalkmrdp evgavmgqlt asnsgdtwlt |
| | | klidmeywla cneeraaqsr fgavmcccgp camyrrsala slldqyetql frgklsdfge |
| | | drhltilmlk agfrteyvpn aivatvvpdt Iksylrqqlr warstfrdtf lalpllrgIn |
| | | pfltfdvvgq nigplllals vvtglahfit tatvpwwtil iiacmtiirc svvalharql |
| | | rflgfvlhtp inlflllplk ayalctlsns dwlsrysape vpvsggkptp iqasgrvtpd |
| | | ctcsgelrrq wshpqfek |
| 3 | SfNodC | mdllgttgavaislyaalstaykgmqaiyalptnttaastpvtgsgappsvdvivpcynedpr |
| | | alsaclasiakqdyagelrvyvvddgsgnrnaiipvhdhyacdprfrfilmpknvgkrkaqiv |
| | | airessgdlvlnvdsdttiapdvvtklalkmyspavgaamgqltasnrsdtwltrlidmeywl |
| | | acneeraaqarfgavmcccgpcamyrrsallllldkyetqlfrgrpsdfgedrhltilmlnag |
| | | frteyvpdaiaatvvpnsmgaylrqqlrwarstfrdtllalrllpgldryltldvigqnlgpl |
| | | llalsvltglaqlaltatvpwstilmiasmtmvrcgvaafrarelrflgfslhtlInvalllp |
| | | lkayalctlsnsdwlsrgspaaapngvkdspephc |
| 4 | RsNodC R346D | mdlIntigig avscyallst ahksmqtlya qpkdqssase dfaflpsvdi ivpcynenph |
| | | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia smtmidcava |
| | | avrarqlrfl vfslhtpinl ffllpmkaya Ictlsnsdwl srsspankts aggehpttea |
| | | saggtsgnat plrrInlard sstvtpagvy sdd |
| 5 | RsNodC R346E | mdlIntigig avscyallst ahksmqtlya qpkdqssase dfaflpsvdi ivpcynenph |
| | | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia smtmiecava |
| | | avrarqlrfl vfslhtpinl ffllpmkaya Ictlsnsdwl srsspankts aggehpttea |
| | | saggtsgnat plrrInlard sstvtpagvy sdd |
| 6 | RsNodC R353E | mdlIntigig avscyallst ahksmqtlya qpkdqssase dfaflpsvdi ivpcynenph |
| | | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia smtmircava |
| | | avearqIrfl vfslhtpinl ffllpmkaya Ictlsnsdwl srsspankts aggehpttea |
| | | saggtsgnat plrrInlard sstvtpagvy sdd |
| 7 | RsNodC R355E | mdlIntigig avscyallst ahksmqtlya qpkdqssase dfaflpsvdi ivpcynenph |
| | | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia smtmircava |
| | | avraeqlrfl vfslhtpinl ffllpmkaya Ictlsnsdwl srsspankts aggehpttea |
| | | saggtsgnat plrrInlard sstvtpagvy sdd |
| 8 | RsNodC R358E | mdlIntigig avscyallst ahksmqtlya qpkdqssase dfaflpsvdi ivpcynenph |
| | | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia smtmircava |
| | | avrarqlefl vfslhtpinl ffllpmkaya Ictlsnsdwl srsspankts aggehpttea |
| | | saggtsgnat plrrInlard sstvtpagvy sdd |
| 9 | RsNodC R346E- | mdlIntigig avscyallst ahksmqtlya qpkdqssase dfaflpsvdi ivpcynenph |
| | R353E | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia smtmiecava |
| | | avearqIrfl vfslhtpinl ffllpmkaya Ictlsnsdwl srsspankts aggehpttea |
| | | saggtsgnat plrrInlard sstvtpagvy sdd |
| 10 | RsNodC R346E- | mdlIntigig avscyallst ahksmqtlya qpkdqssase dfaflpsvdi ivpcynenph |
| | R355E | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia smtmiecava |
| | | avraeqlrfl vfslhtpinl ffllpmkaya Ictlsnsdwl srsspankts aggehpttea |
| | | saggtsgnat plrrInlard sstvtpagvy sdd |
| 11 | RsNodC R346E- | mdlIntigig avscyallst ahksmqtlya qpkdqssase dfaflpsvdi ivpcynenph |
| | R358E | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia smtmiecava |
| | | avrarqlefl vfslhtpinl ffllpmkaya Ictlsnsdwl srsspankts aggehpttea |
| | | saggtsgnat plrrInlard sstvtpagvy sdd |
| 12 | RsNodC R197S- | mdlIntigig avscyallst ahksmqtlya qpkdqssase dfaflpsvdi ivpcynenph |
| | R346E | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqasfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia smtmiecava |
| | | avrarqlrfl vfslhtpinl ffllpmkaya Ictlsnsdwl srsspankts aggehpttea |
| | | saggtsgnat plrrInlard sstvtpagvy sdd |
| 13 | RsNodC R346E- | mdlIntigig avscyallst ahksmqtlya qpkdqssase dfaflpsvdi ivpcynenph |
| | R358S | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia smtmiecava |
| | | avrarqlsfl vfslhtpinl ffllpmkaya Ictlsnsdwl srsspankts aggehpttea |
| | | saggtsgnat plrrInlard sstvtpagvy sdd |
| 14 | RsNodC H298T- | mdlIntigig avscyallst ahksmqtlya qpkdqssase dfaflpsvdi ivpcynenph |
| | R346E | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldtyi tldvigqnlg plllalavlt gvlqvaltat vplwtvmmia smtmiecava |
| | | avrarqlrfl vfslhtpinl ffllpmkaya Ictlsnsdwl srsspankts aggehpttea |
| | | saggtsgnat plrrInlard sstvtpagvy sdd |
| 15 | RsNodC | mdlIntigig avscyallst ahksmqtlya qpkdqssase dfaflpsvdi ivpcynenph |
| | M342S-R346E | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia sstmiecava |
| | | avrarqIrfl vfslhtpinl ffllpmkaya Ictlsnsdwl srsspankts aggehpttea |
| | | saggtsgnat plrrInlard sstvtpagvy sdd |
| 16 | RsNodC R197S | mdlIntigig avscyallst ahksmqtlya qpkdqssase dfaflpsvdi ivpcynenph |
| | | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqasfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia smtmircava |
| | | avrarqlrfl vfslhtpinl ffllpmkaya Ictlsnsdwl srsspankts aggehpttea |
| | | saggtsgnat plrrInlard sstvtpagvy sdd |
| 17 | RsNodC R346S | mdlIntigig avscyallst ahksmqtlya qpkdqssase dfaflpsvdi ivpcynenph |
| | | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia smtmiscava |
| | | avrarqlrfl vfslhtpinl ffllpmkaya Ictlsnsdwl srsspankts aggehpttea |
| | | saggtsgnat plrrInlard sstvtpagvy sdd |
| 18 | RsNodC R346Q | mdlIntigig avscyallst ahksmqtlya qpkdqssase dfaflpsvdi ivpcynenph |
| | | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia smtmiqcava |
| | | avrarqlrfl vfslhtpinl ffllpmkaya Ictlsnsdwl srsspankts aggehpttea |
| | | saggtsgnat plrrInlard sstvtpagvy sdd |
| 19 | RsNodC R346W | mdlIntigig avscyallst ahksmqtlya qpkdqssase dfaflpsvdi ivpcynenph |
| | | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia smtmiwcava |
| | | avrarqlrfl vfslhtpinl ffllpmkaya Ictlsnsdwl srsspankts aggehpttea |
| | | saggtsgnat plrrInlard sstvtpagvy sdd |
| 20 | RsNodC H298T- | mdlIntigig avscyallst ahksmqtlya qpkdqssase dfaflpsvdi ivpcynenph |
| | R346S | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldtyi tldvigqnlg plllalavlt gvlqvaltat vplwtvmmia smtmiscava |
| | | avrarqlrfl vfslhtpinl ffllpmkaya Ictlsnsdwl srsspankts aggehpttea |
| | | saggtsgnat plrrInlard sstvtpagvy sdd |
| 21 | RsNodC | mdlIntigig avscyallst ahksmqtlya qpkdqssase dfaflpsvdi ivpcynenph |
| | M342S-R346S | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia sstmiscava |
| | | avrarqlrfl vfslhtpinl ffllpmkaya Ictlsnsdwl srsspankts aggehpttea |
| | | saggtsgnat plrrInlard sstvtpagvy sdd |
| 22 | RsNodC | mdlIntigig avscyallst ahksmqtlya qpkdqssase dfaflpsvdi ivpcynenph |
| | M342W-R346S | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldhyi tldvigqnlg plllalavlt gvlqvaltat vplwtvmmia swtmiscava |
| | | avrarqlrfl vfslhtpinl ffllpmkaya Ictlsnsdwl srsspankts aggehpttea |
| | | saggtsgnat plrrInlard sstvtpagvy sdd |
| 23 | RsNodC H298T- | mdlIntigig avscyallst ahksmqtlya qpkdqssase dfaflpsvdi ivpcynenph |
| | L302T | tfseclasia nqdyagklrv yvvddgsanr eklervhhty agdprfdfil Irenvgkrka |
| | | qiaairgssg dlvlnvdsds tlasdvvtkl alkmqnpeig aamgqltasn rndtwltrli |
| | | dmeywlacne eraaqarfga vmcccgpcam yrrsallsll dqyesqffrg |
| | | kpsdfgedrh Itilmlkagf rtdyvpdaia atvvpdrmgp ylrqqlrwar stfrdtllal |
| | | rllpgldtyi ttdvigqnlg plllalavlt gvlqvaltat vplwtvmmia smtmircava |
| | | avrarqIrfl vfslhtpinl ffllpmkaya Ictlsnsdwl srsspankts aggehpttea |
| | | saggtsgnat plrrInlard sstvtpagvy sdd |
| 24 | SmNodC R349S | mylldttsta aisiyalllt ayrsmqvlha rpidgpavsa epvetrplpa vdvivpsfne |
| | | dpgilsacla siadqdypge Irvyvvddgs rnreailrvr afysrdprfs fillpenvgk |
| | | rkaqiaaigq ssgdlvlnvd sdstiafdvv sklalkmrdp evgavmgqlt asnsgdtwlt |
| | | klidmeywla cneeraaqsr fgavmcccgp camyrrsala slldqyetql frgklsdfge |
| | | drhltilmlk agfrteyvpn aivatvvpdt Iksylrqqlr warstfrdtf lalpllrgIn |
| | | pfltfdvvgq nigplllals vvtglahfit tatvpwwtil iiacmtiisc svvalharql |
| | | rflgfvlhtp inlflllplk ayalctlsns dwlsrysape vpvsggkptp iqasgrvtpd |
| | | ctcsgelrrq wshpqfek |
| 25 | SmNodC R349E | mylldttsta aisiyalllt ayrsmqvlha rpidgpavsa epvetrplpa vdvivpsfne |
| | | dpgilsacla siadqdypge Irvyvvddgs rnreailrvr afysrdprfs fillpenvgk |
| | | rkaqiaaigq ssgdlvlnvd sdstiafdvv sklalkmrdp evgavmgqlt asnsgdtwlt |
| | | klidmeywla cneeraaqsr fgavmcccgp camyrrsala slldqyetql frgklsdfge |
| | | drhltilmlk agfrteyvpn aivatvvpdt Iksylrqqlr warstfrdtf lalpllrgIn |
| | | pfltfdvvgq nigplllals vvtglahfit tatvpwwtil iiacmtiiec svvalharql |
| | | rflgfvlhtp inlflllplk ayalctlsns dwlsrysape vpvsggkptp iqasgrvtpd |
| | | ctcsgelrrq wshpqfek |
| 26 | SmNodC R200S | mylldttsta aisiyalllt ayrsmqvlha rpidgpavsa epvetrplpa vdvivpsfne |
| | | dpgilsacla siadqdypge Irvyvvddgs rnreailrvr afysrdprfs fillpenvgk |
| | | rkaqiaaigq ssgdlvlnvd sdstiafdvv sklalkmrdp evgavmgqlt asnsgdtwlt |
| | | klidmeywla cneeraaqss fgavmcccgp camyrrsala slldqyetql frgklsdfge |
| | | drhltilmlk agfrteyvpn aivatvvpdt Iksylrqqlr warstfrdtf lalpllrgIn |
| | | pfltfdvvgq nigplllals vvtglahfit tatvpwwtil iiacmtiirc svvalharql |
| | | rflgfvlhtp inlflllplk ayalctlsns dwlsrysape vpvsggkptp iqasgrvtpd |
| | | ctcsgelrrq wshpqfek |
| 27 | SmNodC | mylldttsta aisiyalllt ayrsmqvlha rpidgpavsa epvetrplpa vdvivpsfne |
| | R200S-R349E | dpgilsacla siadqdypge Irvyvvddgs rnreailrvr afysrdprfs fillpenvgk |
| | | rkaqiaaigq ssgdlvlnvd sdstiafdvv sklalkmrdp evgavmgqlt asnsgdtwlt |
| | | klidmeywla cneeraaqss fgavmcccgp camyrrsala slldqyetql frgklsdfge |
| | | drhltilmlk agfrteyvpn aivatvvpdt Iksylrqqlr warstfrdtf lalpllrgIn |
| | | pfltfdvvgq nigplllals vvtglahfit tatvpwwtil iiacmtiiec svvalharql |
| | | rflgfvlhtp inlflllplk ayalctlsns dwlsrysape vpvsggkptp iqasgrvtpd |
| | | ctcsgelrrq wshpqfek |
| 28 | SfNodC R349E | mdllgttgavaislyaalstaykgmqaiyalptnttaastpvtgsgappsvdvivpcynedpr |
| | | alsaclasiakqdyagelrvyvvddgsgnrnaiipvhdhyacdprfrfilmpknvgkrkaqiv |
| | | airessgdlvlnvdsdttiapdvvtklalkmyspavgaamgqltasnrsdtwltrlidmeywl |
| | | acneeraaqarfgavmcccgpcamyrrsallllldkyetqlfrgrpsdfgedrhltilmlnag |
| | | frteyvpdaiaatvvpnsmgaylrqqlrwarstfrdtllalrllpgldryltldvigqnlgpl |
| | | llalsvltglaqlaltatvpwstilmiasmtmvecgvaafrarelrflgfslhtlInvalllp |
| | | lkayalctlsnsdwlsrgspaaapngvkdspephc |
| 29 | SfNodC R349S | mdllgttgavaislyaalstaykgmqaiyalptnttaastpvtgsgappsvdvivpcynedpr |
| | | alsaclasiakqdyagelrvyvvddgsgnrnaiipvhdhyacdprfrfilmpknvgkrkaqiv |
| | | airessgdlvlnvdsdttiapdvvtklalkmyspavgaamgqltasnrsdtwltrlidmeywl |
| | | acneeraaqarfgavmcccgpcamyrrsallllldkyetqlfrgrpsdfgedrhltilmlnag |
| | | frteyvpdaiaatvvpnsmgaylrqqlrwarstfrdtllalrllpgldryltldvigqnlgpl |
| | | llalsvltglaqlaltatvpwstilmiasmtmvscgvaafrarelrflgfslhtlInvalllp |
| | | lkayalctlsnsdwlsrgspaaapngvkdspephc |
| 30 | SmNodC | mylldttsta aisiyalllt ayrsmqvlha rpidgpavsa epvetrplpa vdvivpsfne |
| | H356R | dpgilsacla siadqdypge Irvyvvddgs rnreailrvr afysrdprfs fillpenvgk |
| | | rkaqiaaigq ssgdlvlnvd sdstiafdvv sklalkmrdp evgavmgqlt asnsgdtwlt |
| | | klidmeywla cneeraaqsr fgavmcccgp camyrrsala slldqyetql frgklsdfge |
| | | drhltilmlk agfrteyvpn aivatvvpdt Iksylrqqlr warstfrdtf lalpllrgIn |
| | | pfltfdvvgq nigplllals vvtglahfit tatvpwwtil iiacmtiirc svvalrarql |
| | | rflgfvlhtp inlflllplk ayalctlsns dwlsrysape vpvsggkptp iqasgrvtpd |
| | | ctcsgelrrq wshpqfek |

### EXAMPLES

### Material and Methods

### 1. Chemicals, Oligonucleotides and Molecular Biology

All reagents were purchased from Sigma-Aldrich (Bornem, Belgium), unless stated otherwise. Agarose and ethidium bromide were purchased from Thermo Fisher Scientific (Erembodegem, Belgium). Chitintetraose, -pentaose and -hexaose standards (10 mg) were purchased from Megazyme (The Netherlands). Standard molecular biology procedures were conducted as described by Sambrook et al. (Sambrook and Russell 2001). Qiagen kits (Hilden, Germany) were used for all DNA preparations. Oligonucleotides were purchased from Integrated DNA Technologies (Leuven, Belgium), genes were purchased from Geneart (Thermo Fisher Scientific, Erembodegem, Belgium). Sequencing services were conducted by Macrogen (Amsterdam, The Netherlands).

The sequences of the oligomer chitin synthases (oCHS) were obtained from Rhizobium sp. GRH2 (RsNodC, Genbank access code: AJW76243, Table 1) and from Sinorhizobium meliloti strain 1021 (SmNodC, Genbank access code: AAK65131.1, Table 1). The sequence of the oligomer chitin synthase (oCHS) was obtained from Sinorhizobium fredii USDA 191 (SfNodC, Table 1) through genome walking with degenerate primers (Table 2) and subsequent sequencing.

### 2. Strains

Escherichia coli One Shot Top10 Electrocomp™ (Invitrogen, Carlsbad, California, USA) were used for the construction and maintenance of all plasmids. Escherichia coli K12 MG1655 (code: E. coli sWT) was used as the parent for all strain engineering experiments and was obtained from ATCC. Escherichia coli K12 MG1655 ΔnagZΔchiAΔchbBCARFG (code: E. coli s3KO) was used in growth and production experiments and was produced in house from E. coli sWT. Sinorhizobium fredii USDA 191 was purchased from the BCCM/LMG bacterial culture collection (LMG 6216—Ensifer fredii).

Site directed chromosomal alterations in E. coli was accomplished by homologous recombination mediated by λ-Red recombinase (induced from pKD46) as described by Datsenko and Wanner (Datsenko and Wanner 2000). Linear DNA for homologous recombination was generated by amplifying the FRT flanked antibiotic resistance cassette from the appropriate template (pKD3 or pKD4 for gene deletion). Positive transformants were cured from the antibiotic resistance cassette using FLP recombinase (induced from pCP20). Successful chromosomal integration/deletion was confirmed by colony PCR and subsequent sequencing. All oligonucleotides used are listed in Table 2. A list of all used strains is given in Table 3.

TABLE 2
List of used oligonucleotides

| SEQ ID | | |
|---|---|---|
| No | Alteration | Oligonucleotides (5′-3′) |
| | KO chb | |
| 31 | Fw_KO_chb | GGAATTAATCGCCGGATGCAAGGTTCACGCCGCATCTGGCAA |
| | | ACATCCTCACGTGTAGGCTGGAGCTGCTTC |
| 32 | Rv_KO_chb | GGCTTGCGGAGTGTCTGGCTGACAGATAATCGTCGATGAGG |
| | | GCAGTTTTCATATGAATATCCTCCTTAG |
| 33 | Fw_control_chb | TATTCCCATCCGCGTCTGTTC |
| 34 | Rv_control_chb | AAGCGCCCAATGTATTCCAGG |
| | KO nagZ | |
| 35 | Fw_KO_nagZ | GGCTGGCCGATGACACCTGGCGGCAGCTATTAATAAAACAAT |
| | | AAGGAGAGCAGTCAGCATTACACGTCTTGAGCG |
| 36 | Rv_KO_nagZ | CTGATTCAGACGGGTGCTGATCGCTTTCCAGCGAGCCGAGTC |
| | | CATCAGTTCCTGCCATATGAATATCCTCCTTAG |
| 37 | Fw_control_nagZ | CGGCGCAATTATGGCGTCAG |
| 38 | Rv_control_nagZ | CGGACTGTTAGAGTCAAAACC |
| | KO chiA | |
| 39 | Fw_KO_chiA | TAATTCCTGCGTAGGACTTTTGTTTTGCAGTTTTTACGTCACA |
| | | AGGGCATATGAATATCCTCCTTAG |
| 40 | Rv_KO_chiA | GTAGCCCATTGACAAAAAATGCGGCGATACTGGAAGGTATC |
| | | GCCAACACGTGTAGGCTGGAGCTGGAGCTGCTTC |
| 41 | Fw_control_chiA | GAGACTCCCGTATACTTTCTTC |
| 42 | Rv_control_chiA | CGCCCTTTTTGCATTTGTTG |
| | SmNodC R349S | |
| 43 | SmNodC R349S_fw | GCGTGCATGACCATTATAAGCTGCAGCGTCGTAGCATT |
| | | GCATG |
| 44 | SmNodC_R349S_rv | CATGCAATGCTACGACGCTGCAGCTTATAATGGTCATG |
| | | CACGC |
| | SmNodC R349E | |
| 45 | SmNodC R349E_fw | CCATTATAGAATGCAGCGTCGTAGCATTGCATGCTCGC |
| | | CAACTTAG |
| 46 | SmNodC R349E_rv | GACGCTGCATTCTATAATGGTCATGCACG |
| | SmNodC R200S | |
| 47 | SmNodC R200S_fw | GGCACAGTCTAGCTTCGGTGCTGTTATGTGTTG |
| 48 | SmNodC R200S_rv | CACCGAAGCTAGACTGTGCC |
| | RsNodC R346D | |
| 49 | RsNodC R346D_fw | GACGATGATCGATTGTGCAGTTGCAGCAGTCC |
| 50 | RsNodC R346D_rv | CTGCACAATCGATCATCGTCATTGATGCAATCATCATCA |
| | | C |
| | RsNodC R346E | |
| 51 | RsNodC R346E_fw | CGATGATCGAATGTGCAGTTGCAGC |
| 52 | RsNodC R346E_rv | GCTGCAACTGCACATTCGATCATC |
| | RsNodC R353E | |
| 53 | RsNodC R353E_fw | TTGCAGCAGTCGAAGCACGTCAG |
| 54 | RsNodC R353E_rv | CTGACGTGCTTCGACTGCTGCAACTG |
| | RsNodC R355E | |
| 55 | RsNodC R355E_fw | CCGTGCAGAACAGCTGCGCTTTCTGG |
| 56 | RsNodC R355E_rv | AGCGCAGCTGTTCTGCACGGACTGCTGCAACTG |
| | RsNodC R358E | |
| 57 | RsNodC R358E_fw | GCACGTCAGCTGGAATTTCTGG |
| 58 | RsNodC R358E_rv | CCAGAAATTCCAGCTGACGTG |
| | RsNodC R346E-R353E | |
| 59 | RsNodC R346E_R353E_fw | TTGCAGCAGTCGAAGCACGTCAG |
| 60 | RsNodC R346E_R353E_rv | CTGACGTGCTTCGACTGCTGCAACTG |
| | RsNodC R346E-R355E | |
| 61 | RsNodC R346E_R355E_fw | CCGTGCAGAACAGCTGCGCTTTCTGG |
| 62 | RsNodC R346E_R355E_rv | AGCGCAGCTGTTCTGCACGGACTGCTGCAACTG |
| | RsNodC R346E-R358E | |
| 63 | RsNodC R346E_R358E_fw | GCACGTCAGCTGGAATTTCTGG |
| 64 | RsNodC R346E_R358E_rv | CCAGAAATTCCAGCTGACGTG |
| | RsNodC R197S-R346E | |
| 65 | RsNodC R197S_R346E_fw | GCACAGGCAAGCTTTGGTGCAGTGATG |
| 66 | RsNodC R197S_R346E_rv | GCACCAAAGCTTGCCTGTGCTG |
| | RsNodC R346E-R358S | |
| 67 | RsNodC R346E_R358S_fw | GCACGTCAGCTGAGCTTTCTGG |
| 68 | RsNodC R346E_R358S_rv | CCAGAAAGCTCAGCTGACGTGCACGGACTG |
| | RsNodC H298T-R346E | |
| 69 | RsNodC H298T_R346E_fw | GCCGGGTCTGGATACCTATATTAC |
| 70 | RsNodC H298T_R346E_rv | ATATAGGTATCCAGACCCGGCAGCAGACG |
| | RsNodC M342S-R346E | |
| 71 | RsNodC M342S_R346E_fw | TTGCATCAAGCACGATGATCGAATGTGCAGTTGCAGCA |
| | | GTCC |
| 72 | RsNodC M342S_R346E_rv | GGACTGCTGCAACTGCACATTCGATCATCGTGCTTGAT |
| | | GCAATC |
| | RsNodC R197S | |
| 73 | RsNodC R197S_fw | GCACAGGCAAGCTTTGGTGCAGTGATG |
| 74 | RsNodC R197S_rv | GCACCAAAGCTTGCCTGTGCTG |
| | RsNodC R346S | |
| 75 | RsNodC R346S_fw | AATGACGATGATCAGTTGTGCAGTTGCAGCAGTCCG |
| 76 | RsNodC R346S_rv | CGGACTGCTGCAACTGCACAACTGATCATCGTCATTGAT |
| | | G |
| | RsNodC R346Q | |
| 77 | RsNodC R346Q_fw | GACGATGATCCAGTGTGCAGTTG |
| 78 | RsNodC R346Q_rv | CTGCACACTGGATCATCGTCATTG |
| | RsNodC R346W | |
| 79 | RsNodC R346W_fw | AATGACGATGATCTGGTGTGCAGTTGCAGCAGTCC |
| 80 | RsNodC R346W_rv | GGACTGCTGCAACTGCACACCAGATCATCGTCATT |
| | RsNodC H298T | |
| 81 | RsNodC H298T_fw | GCCGGGTCTGGATACCTATATTAC |
| 82 | RsNodC H298T_rv | ATATAGGTATCCAGACCCGGCAGCAGACG |
| | RsNodC H298T-R346S | |
| 83 | RsNodC H298T_R346S_fw | GCCGGGTCTGGATACCTATATTAC |
| 84 | RsNodC H298T_R346S_rv | ATATAGGTATCCAGACCCGGCAGCAGACG |
| | RsNodC M342S-R346S | |
| 85 | RsNodC M342S_R346S_fw | GATCATCGTGCTTGATGCAATCATCATCACGGTCCACAG |
| 86 | RsNodC M342S_R346S_rv | TTGCATCAAGCACGATGATCAGCTGTGCAG |
| | RsNodC M342W-R346S | |
| 87 | RsNodC M342W_R346S_fw | TGATTGCATCATGGACGATGATCAGTTGTGCAGTTGCA |
| | | GCAGTCCG |
| 88 | RsNodC M342W_R346S_rv | CGGACTGCTGCAACTGCACAACTGATCATCGTCCATGA |
| | | TGCAAT |
| | RsNodC H298T-L302T | |
| 89 | RsNodC H298T_L302T_fw | GGTCTGGATACCTATATTACGACCGACGTTATCG |
| 90 | RsNodC H298T_L302T_rv | AACGTCGGTCGTAATATAGGTATCCAGACCCGGCAGCA |
| | | G |
| 91 | Fw_degenerate primer | GAYGAYGGYTCN |
| 92 | Rv_degenerate primer | CCANCGNAGTTGYTG |
| | SfNodC R349S | |
| 93 | SfNodC R349S_fw | TATGACAATGGTCAGCTGCGGCGTGGC |
| 94 | SfNodC R349S_rv | CGCAGCTGACCATTGTCATAG |
| | SfNodC R349E | |
| 95 | SfNodC R349E_fw | GACAATGGTCGAATGCGGCGTGG |
| 96 | SfNodC R349E_rv | CGCCGCATTCGACCATTGTCATAG |
| | SmNodC R349E-R200S | |
| 97 | SmNodC R349E-R200S_fw | GGCACAGTCTAGCTTCGGTGCTGTTATGTGTTG |
| 98 | SmNodC R349E-R200S_rv | CACCGAAGCTAGACTGTGCC |
| | SmNodC H356R | |
| 99 | SmNodC H356R_fw | CTGCAGCGTCGTAGCATTGCGTGCTCGCCAACTTAG |
| 100 | SmNodC H356R_rv | CTAAGTTGGCGAGCACGCAATGCTACGACGCTGCAG |

TABLE 3
List of all used strains

| code | genotype | Reference |
|---|---|---|
| E. coli sTOP10 | Escherichia coli One Shot TOP10 Electro-comp ™ | Life Technologies |
| E. coli sWT | Escherichia coli K12 MG1655 | ATCC 47076 |
| E. coli s3KO | Escherichia coli K12 MG1655 Δchb ΔchiA ΔnagZ | This study |
| E. coli sSmNodC1 | Escherichia coli s3KO + pCOSA4-SmNodC | This study |
| E. coli sSmNodC2 | Escherichia coli s3KO + pCOSA4-SmNodC R349S | This study |
| E. coli sSmNodC3 | Escherichia coli s3KO + pCOSA4-SmNodC R349E | This study |
| E. coli sSmNodC4 | Escherichia coli s3KO + pCOSA4-SmNodC R200S | This study |
| E. coli sSmNodC5 | Escherichia coli s3KO + pCOSA4-SmNodC H356R | This study |
| E. coli sSmNodC6 | Escherichia coli s3KO + pCOSA4-SmNodC R200S-R349E | This study |
| E. coli sRsNodC1 | Escherichia coli s3KO + pCOSA5-RsNodC | This study |
| E. coli sRsNodC2 | Escherichia coli s3KO + pCOSA5-RsNodC R346D | This study |
| E. coli sRsNodC3 | Escherichia coli s3KO + pCOSA5-RsNodC R346E | This study |
| E. coli sRsNodC4 | Escherichia coli s3KO + pCOSA5-RsNodC R353E | This study |
| E. coli sRsNodC5 | Escherichia coli s3KO + pCOSA5-RsNodC R355E | This study |
| E. coli sRsNodC6 | Escherichia coli s3KO + pCOSA5-RsNodC R358E | This study |
| E. coli sRsNodC7 | Escherichia coli s3KO + pCOSA5-RsNodC R346E-R353E | This study |
| E. coli sRsNodC8 | Escherichia coli s3KO + pCOSA5-RsNodC R346E-R355E | This study |
| E. coli sRsNodC9 | Escherichia coli s3KO + pCOSA5-RsNodC R346E-R358E | This study |
| E. coli sRsNodC10 | Escherichia coli s3KO + pCOSA5-RsNodC R197S-R346E | This study |
| E. coli sRsNodC11 | Escherichia coli s3KO + pCOSA5-RsNodC R346E-R358S | This study |
| E. coli sRsNodC12 | Escherichia coli s3KO + pCOSA5-RsNodC H298T-R346E | This study |
| E. coli sRsNodC13 | Escherichia coli s3KO + pCOSA5-RsNodC R346E-M342S | This study |
| E. coli sRsNodC14 | Escherichia coli s3KO + pCOSA5-RsNodC R197S | This study |
| E. coli sRsNodC15 | Escherichia coli s3KO + pCOSA5-RsNodC R346S | This study |
| E. coli sRsNodC16 | Escherichia coli s3KO + pCOSA5-RsNodC R346Q | This study |
| E. coli sRsNodC17 | Escherichia coli s3KO + pCOSA5-RsNodC R346W | This study |
| E. coli sRsNodC18 | Escherichia coli s3KO + pCOSA5-RsNodC H298T-R346S | This study |
| E. coli sRsNodC19 | Escherichia coli s3KO + pCOSA5-RsNodC M342S-R346S | This study |
| E. coli sRsNodC20 | Escherichia coli s3KO + pCOSA5-RsNodC M342W-R346S | This study |
| E. coli sRsNodC21 | Escherichia coli s3KO + pCOSA5-RsNodC H298T-L302T | This study |
| S. fredii sWT | Sinorhizobium fredii USDA 191 | BCCM/LMG |
| E. coli sSfNodC1 | Escherichia coli s3KO + pCOSA5-SfNodC | This study |
| E. coli sSfNodC2 | Escherichia coli s3KO + pCOSA5-SfNodC R349E | This study |
| E. coli sSfNodC3 | Escherichia coli s3KO + pCOSA5-SfNodC R349S | This study |

### 3. Plasmids

All plasmids used in this study are listed in Table 4. All plasmids were constructed using Circular Polymerase Extension Cloning (CPEC) assembly (J. Quan and Tian 2009). DNA oligonucleotides were purchased from IDT and are listed in Table 2. All E. coli expression vectors, consisting of the nodC gene from Rhizobium sp. GRH2 and Sinorhizobium fredii USDA 191, contained a pBR322 origin of replication (Prentki and Krisch 1982) with an ampicillin resistance marker (Hedges and Jacob 1974).

The pCOSA5 production plasmids were based on the pCXhP14-mKate2 expression vector (origin, antibiotic resistance and P₁₄ promoter and RBS (De Mey et al. 2007; Aerts et al. 2011; Shcherbo et al. 2009)) in which the mKate2 gene was replaced with a gene coding for the oligomer chitin synthases (oCHS) RsNodC and SfNodC, respectively. The pCOSA4 production plasmids, consisting of the nodC gene from Sinorhizobium meliloti strain 1021 are high-copy vectors origination from plasmid pUC57 with a pMB1 origin of replication (ori) and an ampicillin resistance marker (Prentki and Krisch 1982, Hedges and Jacob 1974). These plasmids also carries an operon devoid of any coding sequences, which comprises the constitutive P₂₂ promoter and RBS from Aerts et al. (2011) (De Mey et al. 2007; Aerts et al. 2011; Shcherbo et al. 2009). The coding sequence for the chitin oligosaccharide synthase SmNodC was placed in this operon. Site-directed mutagenesis, one- and multi-site, to introduce specific point mutations was performed as described by Liu et al. (2008).

TABLE 4
Overview of the used, constructed and characterized plasmids

| Plasmid name | Description | Reference |
|---|---|---|
| pKD46 | Helper plasmid for genome | (Datsenko and |
| | modification (λ-Red recombinase) | Wanner 2000) |
| pCP20 | Helper plasmid for genome | (Datsenko and |
| | modification (FLP recombinase) | Wanner 2000) |
| pKD3 | FRT-CmR-FRT | (Datsenko and |
| | | Wanner 2000) |
| pKD4 | FRT-KanR-FRT | (Datsenko and |
| | | Wanner 2000) |
| pCXhP14-mKate2 | pBR322-P₁₄-mKATE2 | (Aerts et al. |
| | | 2011; Shcherbo |
| | | et al. 2009) |
| pCOSA4-SmNodC | pUC-P₂₂-RBS(pCX)-SmnodC | This study |
| pCOSA4-SmNodC R349S | pUC-P₂₂-RBS(pCX)-SmnodC R349S | This study |
| pCOSA4-SmNodC R349E | pUC-P₂₂-RBS(pCX)-SmnodC R349E | This study |
| pCOSA4-SmNodC R200S | pUC-P22-RBS(pCX)-SmnodC R200S | This study |
| pCOSA4-SmNodC R349E-R200S | pUC-P22-RBS(pCX)-SmnodC R349E-R200S | This study |
| pCOSA4-SmNodC H356R | pUC-P22-RBS(pCX)-SmnodC H356R | This study |
| pCOSA5-RsNodC | pBR322-P₁₄-RsnodC | This study |
| pCOSA5-RsNodC R346E | pBR322-P₁₄-RsnodC R346E | This study |
| pCOSA5-RsNodC R346D | pBR322-P₁₄-RsnodC R346D | This study |
| pCOSA5-RsNodC R353E | pBR322-P₁₄-RsnodC R353E | This study |
| pCOSA5-RsNodC R355E | pBR322-P₁₄-RsnodC R355E | This study |
| pCOSA5-RsNodC R358E | pBR322-P₁₄-RsnodC R358E | This study |
| pCOSA5-RsNodC R346S | pBR322-P₁₄-RsnodC R346S | This study |
| pCOSA5-RsNodC R346Q | pBR322-P₁₄-RsnodC R346Q | This study |
| pCOSA5-RsNodC R197S | pBR322-P₁₄-RsnodC R197S | This study |
| pCOSA5-RsNodC R346W | pBR322-P₁₄-RsnodC R346W | This study |
| pCOSA5-RsNodC R346E-R353E | pBR322-P₁₄-RsnodC R346E-R353E | This study |
| pCOSA5-RsNodC R346E-R355E | pBR322-P₁₄-RsnodC R346E-R355E | This study |
| pCOSA5-RsNodC R346E-R358E | pBR322-P₁₄-RsnodC R346E-R358E | This study |
| pCOSA5-RsNodC R197S-R346E | pBR322-P₁₄-RsnodC R197S-R346E | This study |
| pCOSA5-RsNodC R346E-R358S | pBR322-P₁₄-RsnodC R346E-R358S | This study |
| pCOSA5-RsNodC H298T-R346E | pBR322-P₁₄-RsnodC H298T-R346E | This study |
| pCOSA5-RsNodC M342S-R346E | pBR322-P₁₄-RsnodC M342S-R346E | This study |
| pCOSA5-RsNodC H298T-R346S | pBR322-P₁₄-RsnodC H298T-R346S | This study |
| pCOSA5-RsNodC M342S-R346S | pBR322-P₁₄-RsnodC M342S-R346S | This study |
| pCOSA5-RsNodC M342W-R346S | pBR322-P₁₄-RsnodC M342W-R346S | This study |
| pCOSA5-RsNodC H298T-L302T | pBR322-P₁₄-RsnodC H298T-L302T | This study |
| pCOSA5-SfNodC | pBR322-P₁₄-SfnodC | This study |
| pCOSA5-SfNodC R349S | pBR322-P₁₄-SfnodC R349S | This study |
| pCOSA5-SfNodC R349E | pBR322-P₁₄-SfnodC R349E | This study |

### 4. Sequencing

Every constructed plasmid was verified by sequencing. Genetic parts of interest were sequenced upon alteration (Knock-out). All sequencing was performed via sequencing services (Macrogen Inc.).

### 5. Media

Lysogeni broth (LB) medium consisted of 10 g/L tryptone peptone (Difco, Belgium), 5 g/L yeast extract (Difco) and 10 g/L NaCl and was autoclaved for 21 min at 121° C. Luria Bertani Agar (LBA) is similarly composed to LB, be it for the addition of 10 g/L agar. Minimal medium contained 2 g/L NH₄CL, 5 g/L (NH₄)₂SO₄, 3 g/L KH₂PO₄, 7.3 g/L K₂HPO₄, 8.4 g/L MOPS, 0.5 g/L NaCl, 0.5 g/L MgSO₄·7H₂O, and 16.5 g/L glucose·H₂O or 15.3 g/L glycerol as carbon source, 1 mL/L trace element solution and 100 μL/L molybdate solution. Trace element solution consisted of 3.6 g/L FeCl₂·4H₂O, 5 g/L CaCl₂·2H₂O, 1.3 g/L MnCl₂·2H₂O, 0.38 g/L CuCl₂. 2H₂O, 0.5 g/L CoCl₂·6H₂O, 0.94 g/L ZnCl₂, 0.0311 g/L H₃BO₄, 0.4 g/L Na₂EDTA. 2H₂O, 1.01 g/L thiamine·HCl. The molybdate solution contained 0.967 g/L Na₂MoO₄·2H₂O. To avoid Maillard reaction and precipitation during sterilization of the shake flask medium, the glucose and magnesium sulphate were autoclaved separately from the remaining salts. Glucose and magnesium sulphate were autoclaved in a 200 mL solution, the remaining salts in an 800 mL solution. Prior to autoclaving, the latter was set to a pH of 7 with 1 M KOH. After autoclaving, these two solutions were cooled down and mixed. Subsequently, the trace element and molybdate solutions were added filter-sterilized with a bottle top filter (Corning PTFE filter, 0.22 μm). If required, the culture medium was supplemented with appropriate antibiotics. Stock concentrations for antibiotics were 100 mg/mL for spectinomycin, 100 mg/mL for ampicillin, 25 mg/mL for chloramphenicol, and 50 mg/L for kanamycin. Antibiotic stocks were diluted 1000× for cell culture experiments. If required, the culture medium was supplemented with inducers.

### 6. Culture Conditions, Optical Density (OD) Measurements

For strain engineering and plasmid construction strains were grown in lysogeny broth (LB) at 30° C. with shaking (200 rpm, LS-λ AppliTek orbital shaker, Nazareth, Belgium).

For growth experiments, E. coli strains were plated on LBA agar medium with appropriate antibiotics for maintenance and selection of the various plasmids used, incubated for 16 h at 30° C. and a single colony was used for a preculture. For flask experiments, precultures were grown in 50 ml centrifuge tubes containing 10 ml LB with the necessary antibiotic for selection pressure. Pre-cultures were grown overnight (16 h) at 30° C. and 200 rpm (LS-X AppliTek orbital shaker, Nazareth, Belgium) and subsequently, used for 1% inoculation of 100 ml glucose defined medium, i.e. minimal medium in 500 ml shake flasks and grown at 30° C. and 200 rpm (LS-X AppliTek orbital shaker, Nazareth, Belgium). At regular intervals, samples for extracellular metabolites analysis were collected and optical density (OD) at 600 nm is determined. OD was measured at 600 nm using a Jasco V-630Bio spectrophotometer (Easton, UK). Experiments were performed in triplicate (n=3).

### 7. Sample Preparation

For chitotetraose, -pentaose, -hexaose and -heptaose biosynthesis, first 0.1 mL broth was diluted 10 times in physiological water for OD600 measurements in a Jasco V-630Bio spectrophotometer (Easton, UK). Subsequently, 1.5 mL broth was centrifuged at 14000 rpm for 10 min. Pellets were stored at −80° C. until further use. Pellets were resuspended in 250 μL 60% ACN, vortexed and centrifuged at 14000 rpm for 10 min. The supernatant was subsequently applied for COS analysis.

### 8. HPLC-ELSD/ESI-MS Analysis

COS molecules were analyzed on a Waters ACQUITY UPLC (Waters, Milford, MA, USA) or a Shimadzu HPLC system (Shimadzu, Jette, Belgium). Both were connected to an ELSD detector. Chitotetraose, -pentaose, -hexaose and -heptaose were separated by hydrophilic interaction chromatography (HILIC) using an ACQUITY UPLC BEH Amide 1.7 μm column (2.1×100 mm, Waters) and a Kinetix 2.6 μm HILIC 100A column (2.6 μm, 4.6 mm×150 mm; Phenomenex, Utrecht, The Netherlands). Process details, flow rate and elution profile are summarised in table 5 for UPLC and table 6 for HPLC analysis.

TABLE 5
UPLC details and elution profile for COS
analysis. ACN stands for acetonitrile

| | | Method | | |
|---|---|---|---|---|
| | | Time | | |
| Details | | (min) | % A | % B |
| Column: | Acquity UPLC BEH Amide | 0 | 20 | 80 |
| Column | 45° C. | 2 | 20 | 80 |
| temperature: | | 7 | 50 | 50 |
| ELSD detector: | 40° C., Gain 400 | 7.26 | 65 | 35 |
| Eluent A: | 10 mM NH₄-formate in | 7.75 | 65 | 35 |
| | H₂O + 0.1% formic acid | 8.8 | 20 | 80 |
| Eluent B: | 100% ACN + 0.1% formic acid | 10 | 20 | 80 |
| Injection volume: | 1 μl | | | |
| Flow rate | 450 | | | |
| (mL/min): | | | | |

TABLE 6
HPLC details and elution profile for COS
analysis. ACN stands for acetonitrile

| | | Method | | |
|---|---|---|---|---|
| | | Time | | |
| Details | | (min) | % A | % B |
| Column: | Kinetex 2.6 μm HILIC | 0-20 | 15 | 85 |
| Column | 35° C. | 20-27 | 45 | 55 |
| temperature: | | 27-30 | 50 | 50 |
| ELSD detector: | 45° C., Gain 7 | 30-40 | 15 | 85 |
| Eluent A: | 10 mM NH₄-formate in | | | |
| | H₂O + 0.1% formic acid | | | |
| Eluent B: | 100% ACN + 0.1% formic acid | | | |
| Injection volume: | 10 μl | | | |
| Flow rate | 600 | | | |
| (mL/min): | | | | |

### 9. Data and Statistical Analysis

Final glycan concentrations were determined based on a calibration curve, and were corrected for biomass by OD₆₀₀ measurements in order to overcome influences that were caused by the differences in culturing methods. Chromatogram analysis was performed using the Openchrom 1.1.0 software package.

All data analysis was performed using pandas (www.pandas.pydata.org). Pairwise comparisons between different strains were done by a two-sided T-test using the scipy.stats package in Python. A significance level of 0.05 was applied. All given COS production profile of the mutant enzymes differ significantly from the COS production profile of the respectively wild-type mutant, except if it is specifically stated.

### Results

### Example 1—Evaluation of Mutation R349S in SmNodC on COS Production

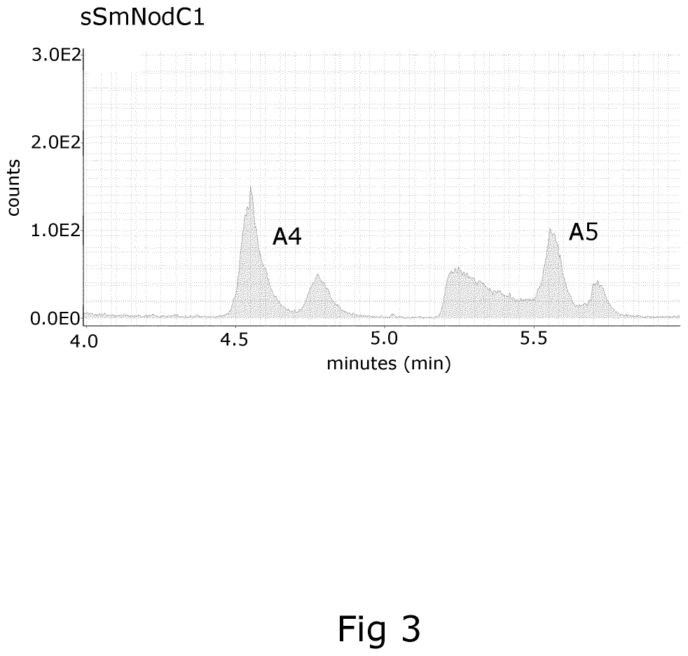

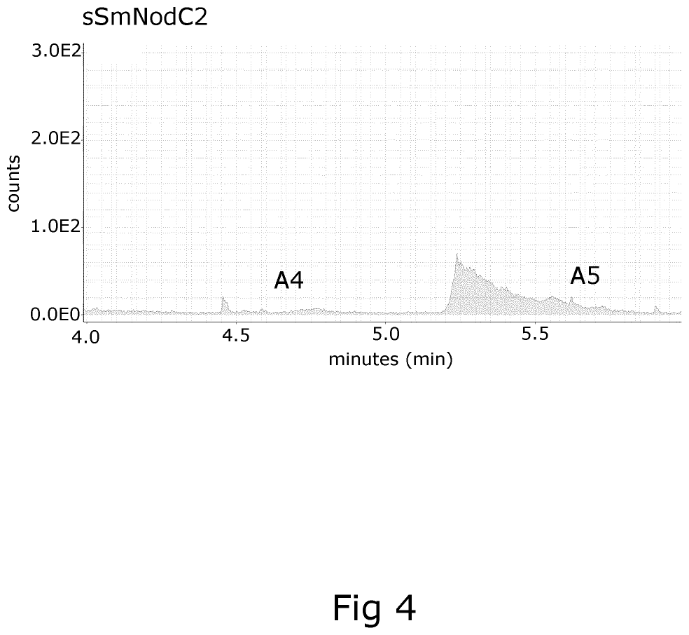

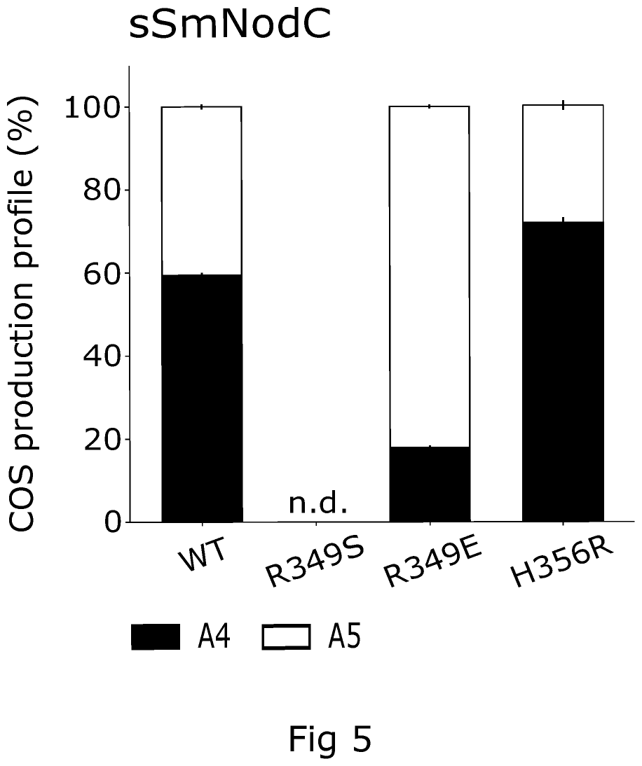

To evaluate the suggestion of Dorfmueller et al. (2014) that the predicted product-binding site for SmNodC is limited by amino acid Arg-349 in the transmembrane helix, to five binding sites, whereas the cellulose synthase structure forms a transport channel through the membrane formed by the transmembrane domains (Ser-459) (Dorfmueller et al. 2014), mutation Arg-349-Ser (R349S) was created in pCOSA4-SmNodC yielding plasmid pCOSA4-SmNodC R349S. Next, E. coli 3KO was transformed with pCOSA4-SmNodC (SEQ ID No 2) and pCOSA4-SmNodC R349S (SEQ ID No 24), respectively yielding E. coli sSmNodC1 and E. coli sSmNodC2. These metabolically engineered strains were grown in minimal medium with glucose. The chromatograms of COS production in E. coli sSmNodC1-2 at 24 h are depicted in FIGS. 3 and 4, respectively. The COS production profile of E. coli sSmNodC1-2 at 24 h are depicted in FIG. 5. sSmNodC1 produces fully acetylated chitintetraose chitinpentaose, chitinhexaose and chitinheptaose with a ratio of 60/40/0/0. In strain sSmNodC2 almost no detectable production of COS could be observed. Introducing the hypothesized shorter side chain amino acids, i.e. Arg-349-Ser, in SmNodC yields an almost non-functional enzyme. Moreover, the COS product profile depicted no shift towards chitin oligosaccharides with a DP higher than four.

### Example 2—Evaluation of a Substitution of a Positively Charged Amino Acid Histidine (H) Present Along One of the Two Transmembrane Helices in SmNodC in to Another Positively Charged Amino on COS Production

The positively charged amino acid arginine at amino acid position 356 (His-356) in SmNodC was substituted in to a positively charged amino acid, i.e. arginine (R), yielding mutation His-356-Arg (H356R). This mutation was created in pCOSA4-SmNodC yielding plasmid pCOSA4-SmNodC H356R.

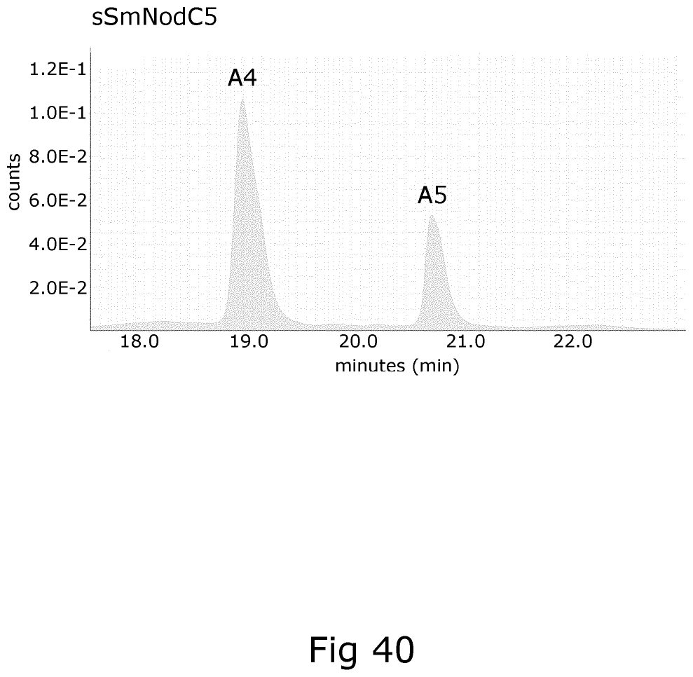

Next, E. coli 3KO was transformed with pCOSA4-SmNodC (SEQ ID No 2) and pCOSA4-SmNodC H356R (SEQ ID No 30), respectively, yielding E. coli sSmNodC1 and E. coli sSmNodC5, respectively. These metabolically engineered strains were grown in minimal medium with glucose. The chromatograms of COS production in E. coli sSmNodC1 and E. coli sSmNodC5 at 24 h are depicted in FIG. 3 and FIG. 40, respectively. The COS production profile of E. coli sSmNodC1 and E. coli sSmNodC5 at 24 h are depicted in FIG. 5. Upon substituting a positively charged amino acid arginine present along one of the two transmembrane helices in SmNodC in to another positively charged amino acid, the fraction of the produced chitin oligosaccharides did not significantly shifted towards chitin oligosaccharides with an increased DP compared to the fraction of the produced chitin oligosaccharides of the wild-type SmNodC. sSmNodC1 produces fully acetylated chitintetraose, chitinpentaose, chitinhexaose and chitinheptaose with a ratio of 60/40/0/0. sSmNodC5 produces fully acetylated chitintetraose, chitinpentaose, chitinhexaose and chitinheptaose with a ratio of 70/30/0/0.

### Example 3—Evaluation of a Substitution of an Arginine (R) Present Along One of the Two Transmembrane Helices in SmNodC in to a Negatively Charged Amino Acid on COS Production

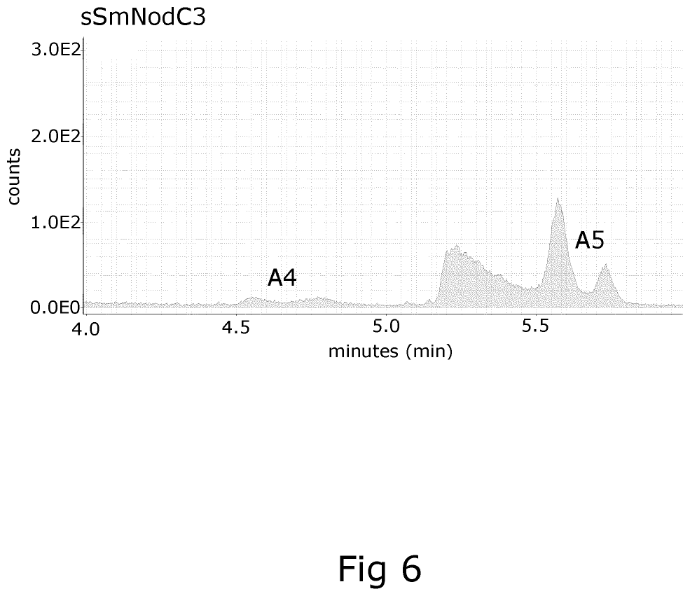

The positively charged amino acid arginine at amino acid position 349 (Arg-349) in SmNodC was substituted in to a negatively charged amino acid, i.e. glutamic acid (E), yielding mutation Arg-349-Glu (R349E). This mutation was created in pCOSA4-SmNodC yielding plasmid pCOSA4-SmNodC R349E. Next, E. coli 3KO was transformed with pCOSA4-SmNodC (SEQ ID No 2) and pCOSA4-SmNodC R349E (SEQ ID No 25), respectively, yielding E. coli sSmNodC1 and E. coli sSmNodC3, respectively. These metabolically engineered strains were grown in minimal medium with glucose. The chromatograms of COS production in E. coli sSmNodC1 and E. coli sSmNodC3 at 24 h are depicted in FIG. 3 and FIG. 6, respectively. The COS production profile of E. coli sSmNodC1 and E. coli sSmNodC3 at 24 h are depicted in FIG. 5. Upon substituting a positively charged amino acid arginine present along one of the two transmembrane helices in SmNodC in to a negatively charged amino acid, the fraction of the produced chitin oligosaccharides significantly shifted towards chitin oligosaccharides with an increased DP compared to the fraction of the produced chitin oligosaccharides of the wild-type SmNodC regardless the bulkiness of the negatively charged amino acid. sSmNodC1 produces fully acetylated chitintetraose, chitinpentaose, chitinhexaose and chitinheptaose with a ratio of 60/40/0/0. sSmNodC3 produces fully acetylated chitintetraose, chitinpentaose, chitinhexaose and chitinheptaose with a ratio of 20/80/0/0.

### Example 4—Evaluation of a Substitution of an Arginine (R) Present Along One of the Two Transmembrane Helices in RsNodC in to a Negatively Charged Amino Acid on COS Production

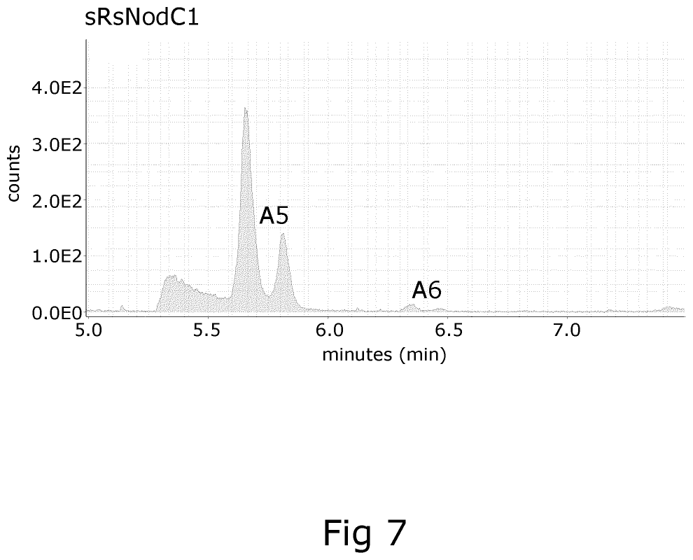

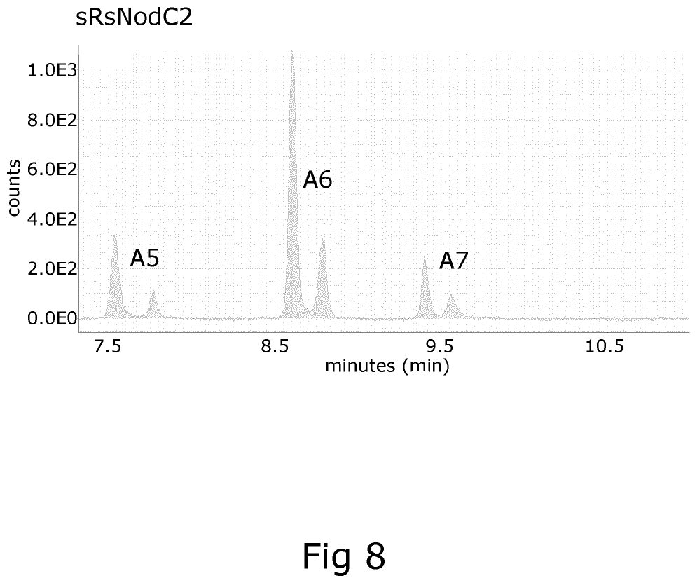

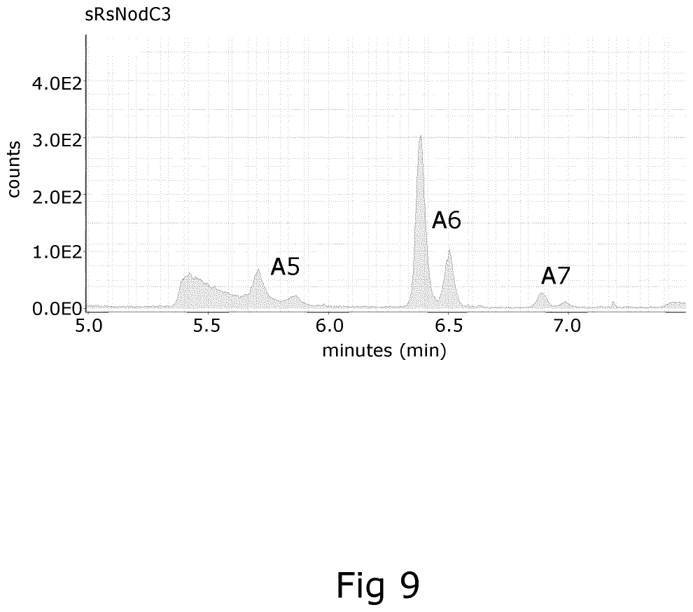

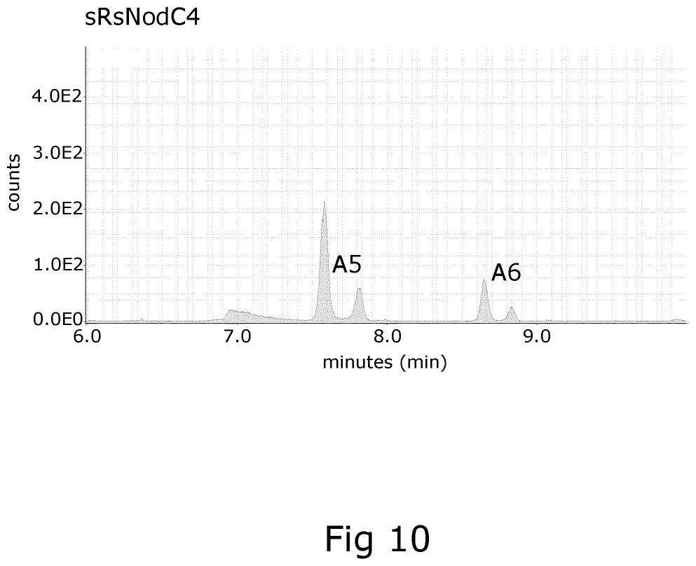

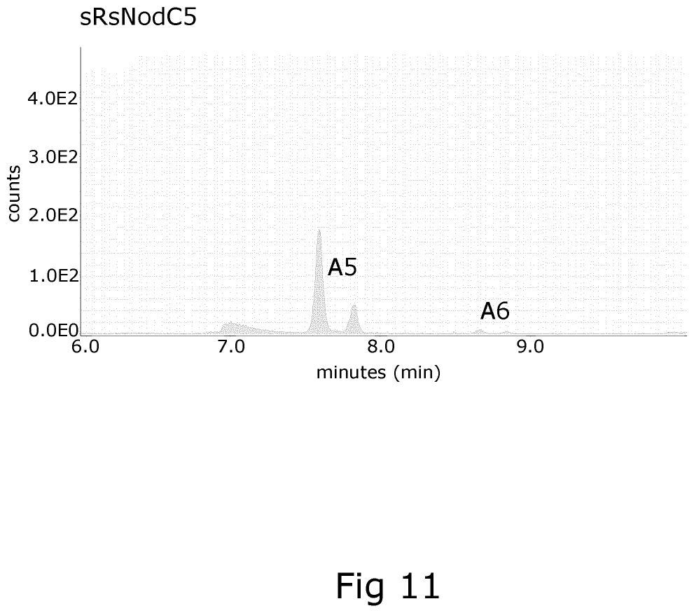

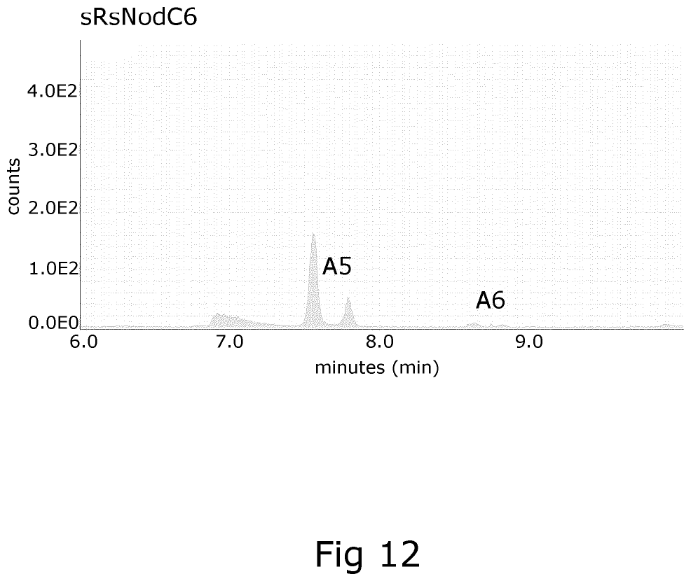

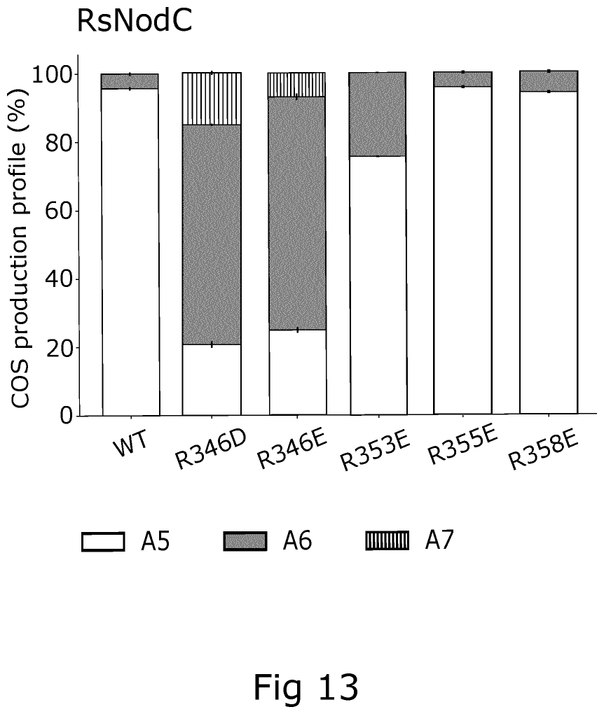

The positively charged amino acid arginine at amino acid position 346 (Arg-346) in RsNodC was substituted in to a negatively charged amino acid, i.e. aspartic acid (D) and glutamic acid (E), yielding mutations Arg-346-Asp (R346D) and Arg-346-Glu (R346E), respectively. The positively charged amino acid arginine at amino acid position 353 (Arg-353) in RsNodC was substituted in to a negatively charged amino acid glutamic acid (E), yielding mutation Arg-353-Glu (R353E). The positively charged amino acid arginine at amino acid position 355 (Arg-355) in RsNodC was substituted in to a negatively charged amino acid glutamic acid (E), yielding mutation Arg-355-Glu (R355E). The positively charged amino acid arginine at amino acid position 358 (Arg-358) in RsNodC was substituted in to a negatively charged amino acid glutamic acid (E), yielding mutation Arg-358-Glu (R355E). All mutations were created in pCOSA5-RsNodC yielding plasmid pCOSA5-RsNodC R346D, pCOSA5-RsNodC R346E, pCOSA5-RsNodC R353E, pCOSA5-RsNodC R355E, pCOSA5-RsNodC R358E. Next, E. coli 3KO was transformed with pCOSA5-RsNodC (SEQ ID No 1), pCOSA5-RsNodC R346D (SEQ ID No 4), pCOSA5-RsNodC R346E (SEQ ID No 5), pCOSA5-RsNodC R353E (SEQ ID No 6), pCOSA5-RsNodC R355E (SEQ ID No 7), and pCOSA5-RsNodC R358E (SEQ ID No 8), respectively yielding E. coli sRsNodC1, E. coli sRsNodC2, E. coli sRsNodC3, E. coli sRsNodC4, E. coli sRsNodC5 and E. coli sRsNodC6, respectively. These metabolically engineered strains were grown in minimal medium with glucose. The chromatograms of COS production in E. coli sRsNodC1-6 at 24 h are depicted in FIG. 7-12. The COS production profile of E. coli sRsNodC1-6 at 24 h are depicted in FIG. 13. Upon substituting a positively charged amino acid arginine present along one of the two transmembrane helices in RsNodC in to a negatively charged amino acid, the fraction of the produced chitin oligosaccharides shifted towards chitin oligosaccharides with an increased DP compared to the fraction of the produced chitin oligosaccharides of the wild-type RsNodC. E. coli strains sRsNodC2 and sRsNodC3, carrying pCOSA5-RsNodC R346D and pCOSA5-RsNodC R346E, respectively, depicted significant increased production of chitin oligosaccharides with an DP of six and seven, regardless the bulkiness of the negatively charged amino acid. sRsNodC1 produces fully acetylated chitintetraose, chitinpentaose, chitinhexaose and chitinheptaose with a ratio of 0/96/4/0. sRsNodC2 produces fully acetylated chitintetraose, chitinpentaose, chitinhexaose and chitinheptaose with a ratio of 0/20/65/15. sRsNodC3 produces fully acetylated chitintetraose, chitinpentaose, chitinhexaose and chitinheptaose with a ratio of 0/25/70/5 sRsNodC4 produces fully acetylated chitintetraose, chitinpentaose, chitinhexaose and chitinheptaose with a ratio of 0/70/30/0. sRsNodC5 produces fully acetylated chitintetraose, chitinpentaose, chitinhexaose and chitinheptaose with a ratio of 0/94/6/0. sRsNodC6 produces fully acetylated chitintetraose, chitinpentaose, chitinhexaose and chitinheptaose with a ratio of 0/93/7/0.

### Example 5—Evaluation of a Substitution of an Arginine (R) Present Along One of the Two Transmembrane Helices in SfNodC in to a Negatively Charged Amino Acid on COS Production

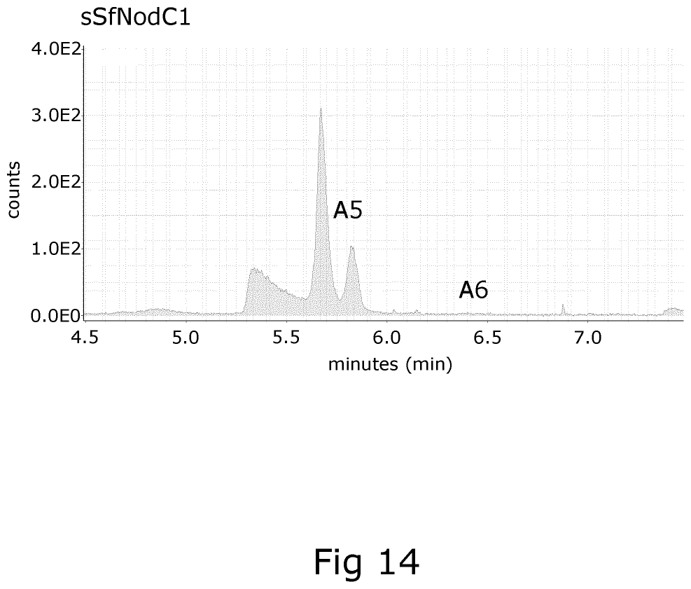

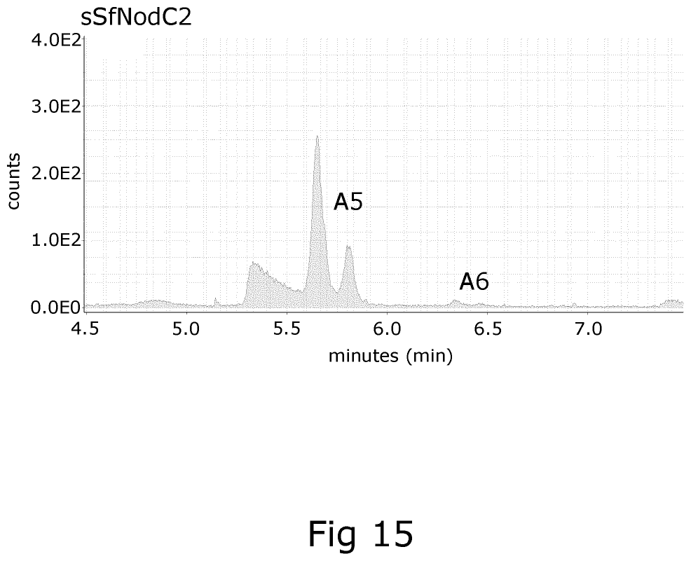

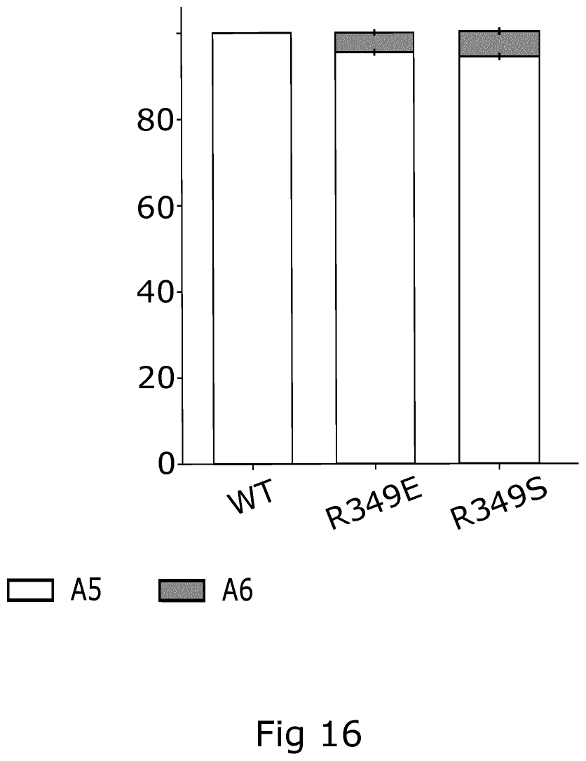

The positively charged amino acid arginine at amino acid position 349 (Arg-349) in SfNodC was substituted in to a negatively charged amino acid, i.e. glutamic acid (E), yielding mutation Arg-349-Glu (R349E). This mutation was created in pCOSA5-SfNodC yielding plasmid pCOSA5-SfNodC R349E. Next, E. coli 3KO was transformed with pCOSA5-SfNodC (SEQ ID No 3) and pCOSA5-SfNodC R349E (SEQ ID No 28), respectively, yielding E. coli sSfNodC1 and E. coli sSfNodC2, respectively. These metabolically engineered strains were grown in minimal medium with glucose. The chromatograms of COS production in E. coli sSfNodC1 and E. coli sSfNodC2 at 24 h are depicted in FIG. 14 and FIG. 15, respectively. The COS production profile of E. coli sSfNodC1 and E. coli sSfNodC2 at 24 h are depicted in FIG. 16. Upon substituting a positively charged amino acid arginine present along one of the two transmembrane helices in SfNodC in to a negatively charged amino acid, the fraction of produced chitin oligosaccharides significantly shifted towards chitin oligosaccharides with an increased DP compared to the fraction of produced chitin oligosaccharides of the wild-type SfNodC. E. coli strains sSfNodC2, carrying pCOSA5-SfNodC R349E, depicted significant increased production of chitin oligosaccharides with an DP of six, regardless the bulkiness of the negatively charged amino acid. sSfNodC1 produces fully acetylated chitintetraose, chitinpentaose, chitinhexaose and chitinheptaose with a ratio of 0/100/0/0. sSfNodC2 produces fully acetylated chitintetraose, chitinpentaose, chitinhexaose and chitinheptaose with a ratio of 0/95/5/0.

### Example 6—Evaluation of a Substitution of an Arginine (R) Present Along One of the Two Transmembrane Helices in SfNodC in to a Neutral Amino Acid on COS Production

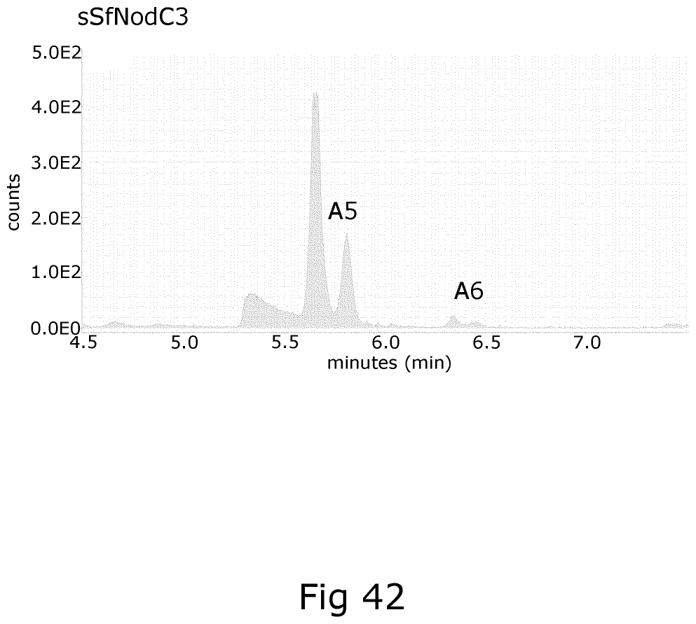

The positively charged amino acid arginine at amino acid position 349 (Arg-349) in SfNodC was substituted in to a neutral amino acid, i.e. serine (S), yielding mutation Arg-349-S(R349S). This mutation was created in pCOSA5-SfNodC yielding plasmid pCOSA5-SfNodC R349S. Next, E. coli 3KO was transformed with pCOSA5-SfNodC (SEQ ID No 3) and pCOSA5-SfNodC R349S (SEQ ID No 29), respectively, yielding E. coli sSfNodC1 and E. coli sSfNodC3, respectively. These metabolically engineered strains were grown in minimal medium with glucose. The chromatograms of COS production in E. coli sSfNodC1 and E. coli sSfNodC3 at 24 h are depicted in FIG. 14 and FIG. 42, respectively. The COS production profile of E. coli sSfNodC1 and E. coli sSfNodC3 at 24 h are depicted in FIG. 16. Upon substituting a positively charged amino acid arginine present along one of the two transmembrane helices in SfNodC in to a neutral amino acid, the fraction of produced chitin oligosaccharides significantly shifted towards chitin oligosaccharides with an increased DP compared to the fraction of produced chitin oligosaccharides of the wild-type SfNodC. E. coli strains sSfNodC3, carrying pCOSA5-SfNodC R349S, depicted significant increased production of chitin oligosaccharides with an DP of six, regardless the bulkiness of the neutral amino acid. sSfNodC1 produces fully acetylated chitintetraose, chitinpentaose, chitinhexaose and chitinheptaose with a ratio of 0/100/0/0. sSfNodC3 produces fully acetylated chitintetraose, chitinpentaose, chitinhexaose and chitinheptaose with a ratio of 0/95/5/0.

### Example 7—Evaluation of a Substitution of an Arginine (R) Present Along One of the Two Transmembrane Helices in RsNodC in to a Negatively Charged Amino Acid Combined with an Additional Substitution of Another Positively Charged Amino Acid Present Along One of the Two Transmembrane Helices in RsNodC in to a Negatively Charged or Neutral Amino Acid on COS Production

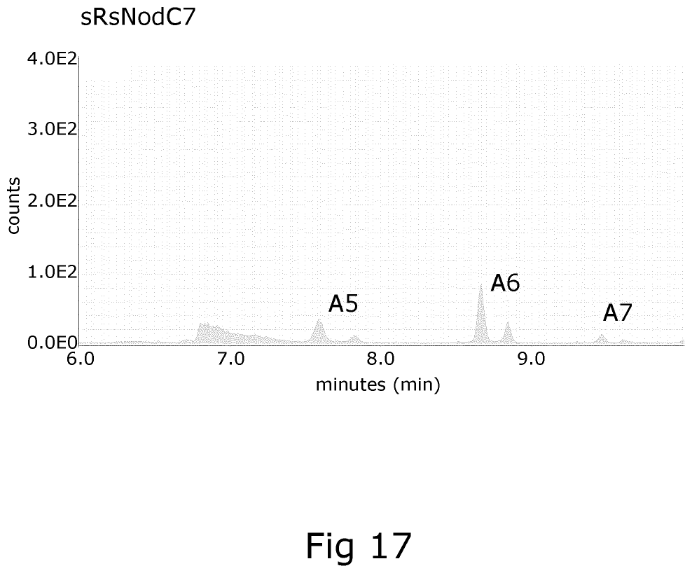

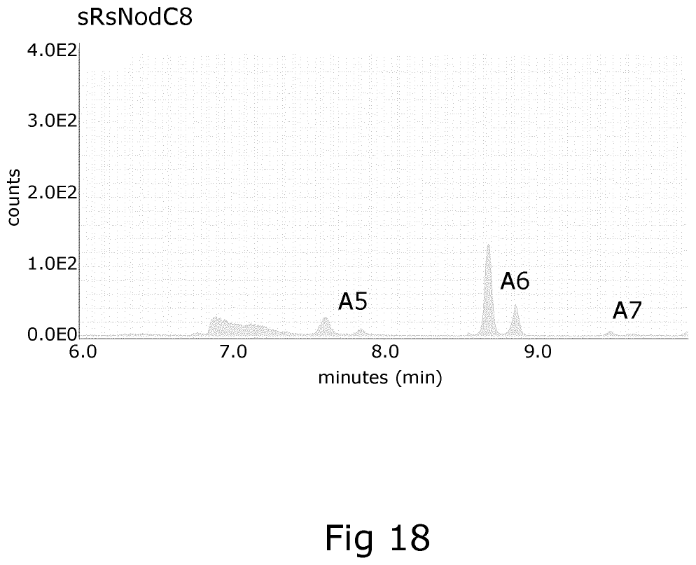

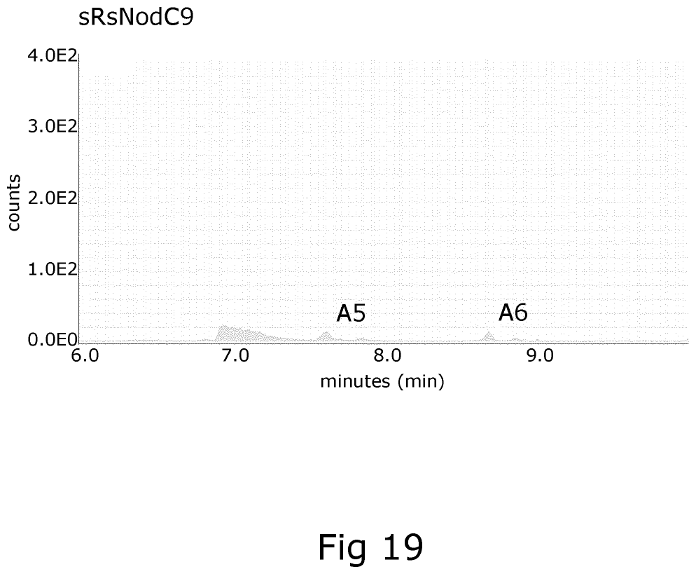

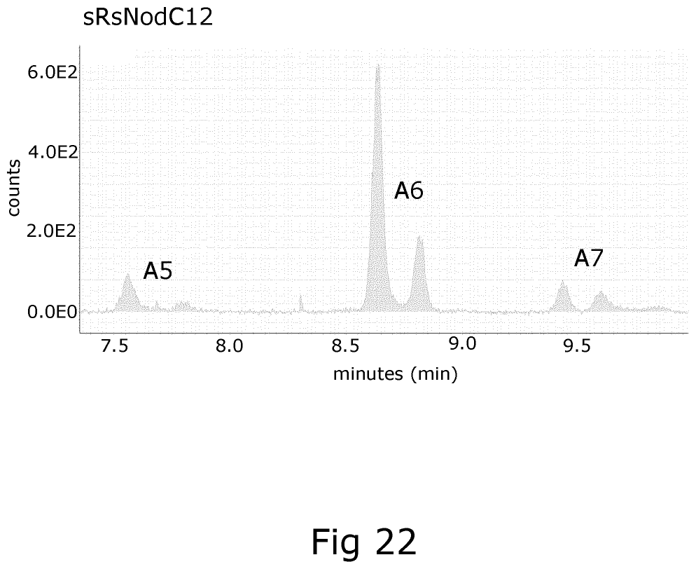

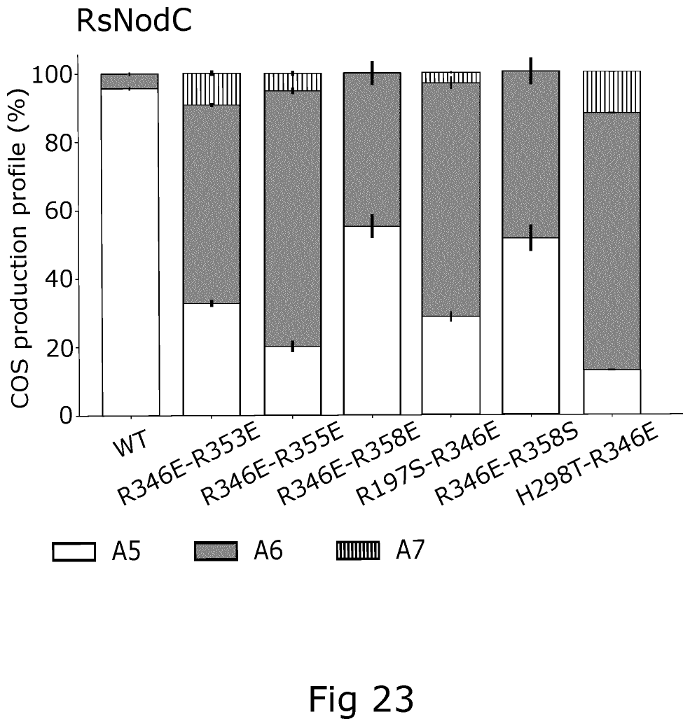

The positively charged amino acid arginine at amino acid position 346 (Arg-346) in RsNodC was substituted in to a negatively charged amino acid, i.e. glutamic acid (E), yielding mutations Arg-346-Glu (R346E). Additionally, the positively charged amino acid arginine at amino acid position 353 (Arg-353) or amino acid position 355 (Arg-355) or amino acid position 358 (Arg-358) in RsNodC was substituted in to a negatively charged amino acid, i.e. glutamic acid (E), or the positively charged amino acid arginine at amino acid position 197 (Arg-197) or amino acid position 358 (Arg-358) in RsNodC was substituted in to a neutral amino acid serine (S), or the positively charged amino acid histidine at amino acid position 298 (His-298) in RsNodC was substituted in to a neutral amino acid threonine (T), yielding the combined mutations Arg-346-Glu-Arg-353-Glu (R346E-R353E), Arg-346-Glu-Arg-355-Glu (R346E-R355E), Arg-346-Glu-Arg-358-Glu (R346E-R358E), Arg-197-Ser-Arg-346-Glu (R197S-R346E), Arg-346-Glu-Arg-358-Ser (R346E-R358S), His-298-Thr-Arg-346-Glu-(H298T-R346E), respectively. All mutations were created in pCOSA5-RsNodC yielding plasmid pCOSA5-RsNodC R346E-R353E, pCOSA5-RsNodC R346E-R355E, pCOSA5-RsNodC R346E-R358E, pCOSA5-RsNodC R197S-R346E, pCOSA5-RsNodC R346E-R358S, pCOSA5-RsNodC H298T-R346E, respectively. Next, E. coli 3KO was transformed with pCOSA5-RsNodC (SEQ ID No 1), pCOSA5-RsNodC R346E-R353E (SEQ ID No 9), pCOSA5-RsNodC R346E-R355E (SEQ ID No 10), pCOSA5-RsNodC R346E-R358E (SEQ ID No 11), pCOSA5-RsNodC R197S-R346E (SEQ ID No 12), pCOSA5-RsNodC R346E-R358S (SEQ ID No 13), pCOSA5-RsNodC H298T-R346E (SEQ ID No 14) yielding E. coli sSRsNodC1 and E. coli sRsNodC7, E. coli sRsNodC8, E. coli sRsNodC9, E. coli sRsNodC10, E. coli sRsNodC11 and E. coli sRsNodC12, respectively. These metabolically engineered strains were grown in minimal medium with glucose. The chromatograms of COS production in E. coli sRsNodC1 and E. coli sRsNodC7-12 at 24 h are depicted in FIG. 7 and FIG. 17-22, respectively. The COS production profile E. coli sRsNodC1 and E. coli sRsNodC7-12 at 24 h are depicted in FIG. 23. Upon combining a substitution of a positively charged amino acid arginine present along one of the two transmembrane helices in RsNodC in to a negatively charged amino acid with an additional substitution of another positively charged amino acid present along one of the two transmembrane helices in RsNodC in to a negatively charged or neutral amino acid, the fraction of produced chitin oligosaccharides significantly shifted towards chitin oligosaccharides with an increased DP compared to the fraction of produced chitin oligosaccharides of the wild-type RsNodC. E. coli strains sRsNodC7, sRsNodC8, sRsNodC10 and sRsNodC12, carrying pCOSA5-RsNodC R346E-R353E, pCOSA5-RsNodC R346E-R355E, pCOSA5-RsNodC R197S-R346E, and pCOSA5-RsNodC H298T-R346E, respectively, depicted significant increased production of chitin oligosaccharides with a DP of six and seven, regardless of the bulkiness of the negatively charged amino acid. sRsNodC1 produces fully acetylated chitintetraose, chitinpentaose, chitinhexaose and chitinheptaose with a ratio of 0/96/4/0. sRsNodC7 produces fully acetylated chitintetraose, chitinpentaose, chitinhexaose and chitinheptaose with a ratio of 0/30/60/10. sRsNodC8 produces fully acetylated chitintetraose, chitinpentaose, chitinhexaose and chitinheptaose with a ratio of 0/20/70/10. sRsNodC9 produces fully acetylated chitintetraose, chitinpentaose, chitinhexaose and chitinheptaose with a ratio of 0/50/50/0. sRsNodC10 produces fully acetylated chitintetraose, chitinpentaose, chitinhexaose and chitinheptaose with a ratio of 0/30/65/5. sRsNodC11 produces fully acetylated chitintetraose, chitinpentaose, chitinhexaose and chitinheptaose with a ratio of 0/50/50/0. sRsNodC12 produces fully acetylated chitintetraose, chitinpentaose, chitinhexaose and chitinheptaose with a ratio of 0/10/80/10.

### Example 8—Evaluation of a Substitution of an Arginine (R) Present Along One of the Two Transmembrane Helices in RsNodC in to a Negatively Charged Amino Acid Combined with a Substitution of Methionine (M) Present Along One of the Two Transmembrane Helices in RsNodC in to a Serine (S) on COS Production

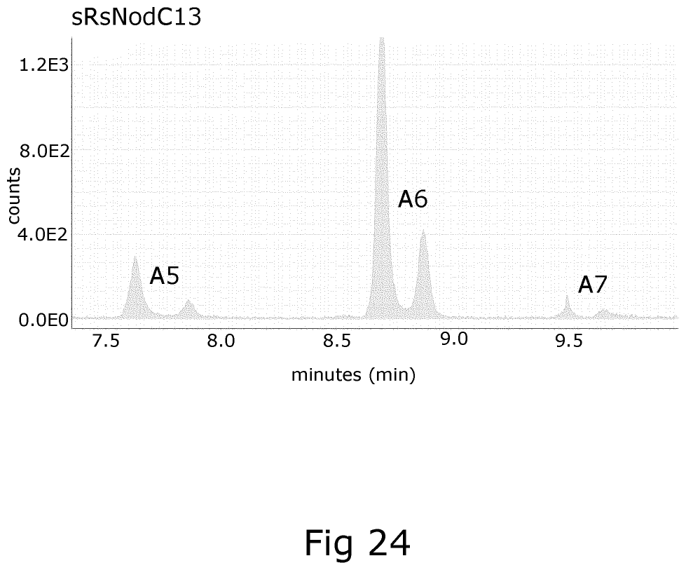

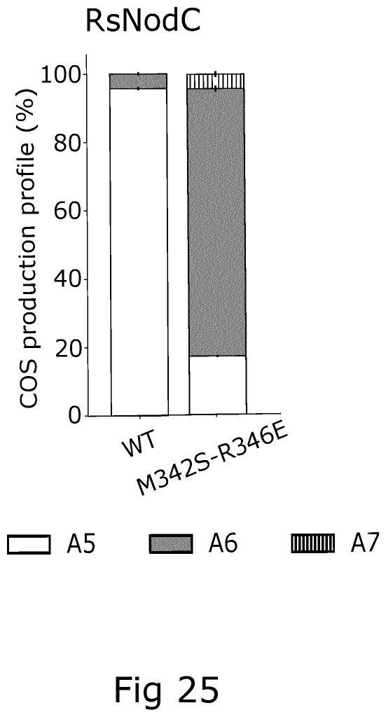

The positively charged amino acid arginine at amino acid position 346 (Arg-346) in RsNodC was substituted in to a negatively charged amino acid, i.e. glutamic acid (E), yielding mutations Arg-346-Glu (R346E). Additionally, the amino acid methionine at amino acid position 342 (Met-342) was substituted in to a serine (S) yielding the combined mutation Met-342-Ser-Arg-346-Glu (M342S-R346E). All mutations were created in pCOSA5-RsNodC yielding plasmid pCOSA5-RsNodC M342S-R346E. Next, E. coli 3KO was transformed with pCOSA5-RsNodC (SEQ ID No 1) and pCOSA5-RsNodC M342S-R346E (SEQ ID No 15), yielding E. coli sRsNodC1 and E. coli sRsNodC13, respectively. These metabolically engineered strains were grown in minimal medium with glucose. The chromatograms of COS production in E. coli sRsNodC1 and E. coli sRsNodC13 at 24 h are depicted in FIG. 7 and FIG. 24, respectively. The COS production profile E. coli sRsNodC1 and E. coli sRsNodC13 at 24 h are depicted in FIG. 25. Upon combining a substitution of a positively charged amino acid arginine present along one of the two transmembrane helices in RsNodC in to a negatively charged amino acid with an additional substitution of a methionine (M) present along one of the two transmembrane helices in RsNodC in to a serine (S), the fraction of the produce chitin oligosaccharides significantly shifted towards chitin oligosaccharides with an increased DP compared to the fraction of the produce chitin oligosaccharides of the wild-type RsNodC. Significantly increased production of chitin oligosaccharides with a DP of six and seven could be observed, regardless of the bulkiness of the negatively charged amino acid. sRsNodC1 produces fully acetylated chitintetraose, chitinpentaose, chitinhexaose and chitinheptaose with a ratio of 0/96/4/0. sRsNodC13 produces fully acetylated chitintetraose, chitinpentaose, chitinhexaose and chitinheptaose with a ratio of 0/20/65/5.

### Example 9—Evaluation of a Substitution of an Arginine (R) Present Along One of the Two Transmembrane Helices in RsNodC in to a Neutral Amino Acid on COS Production

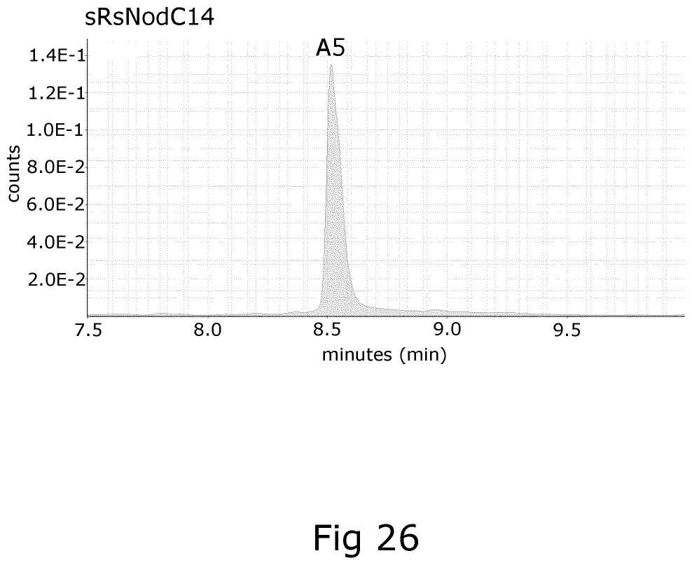

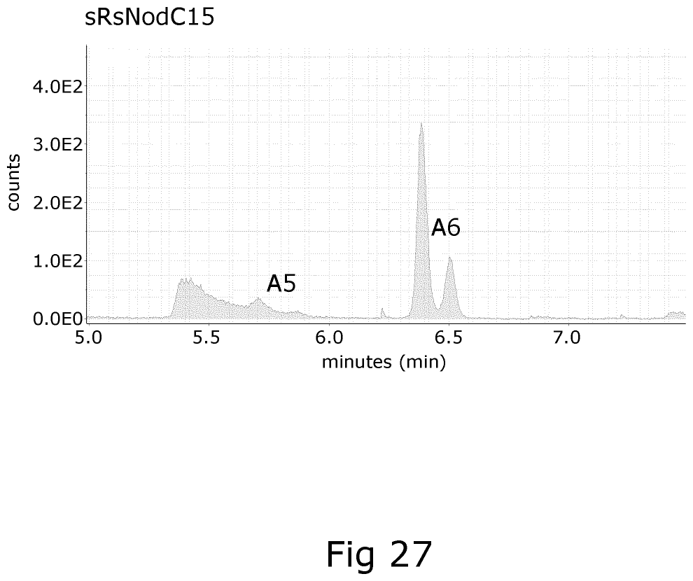

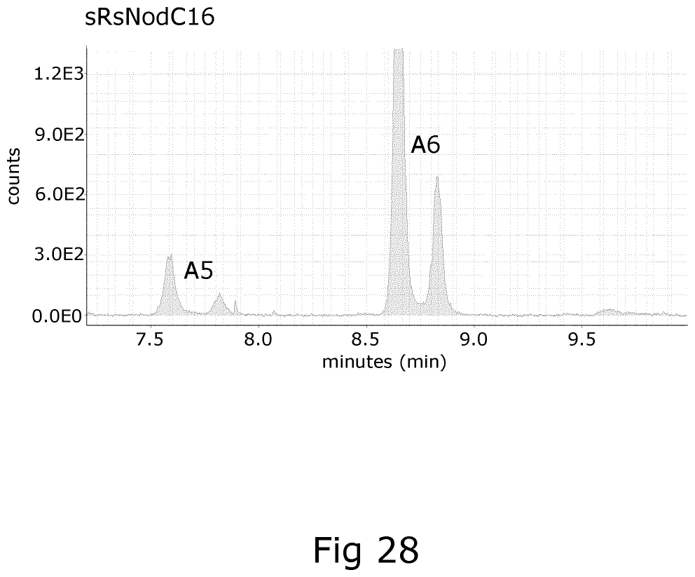

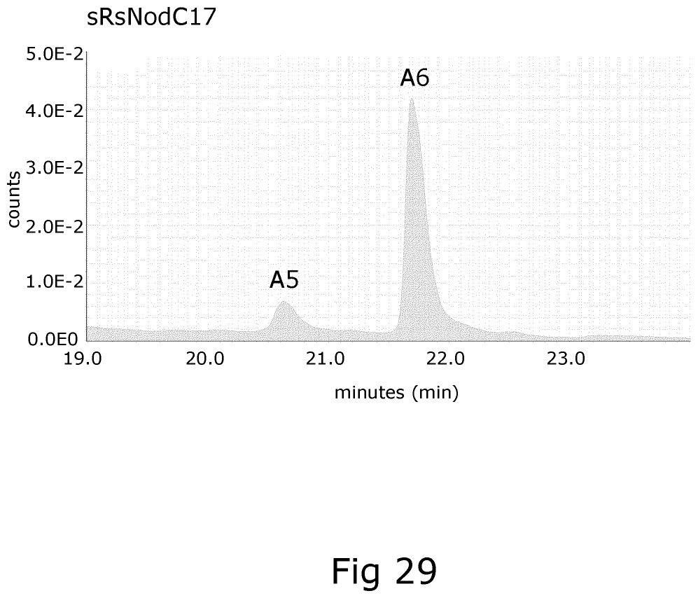

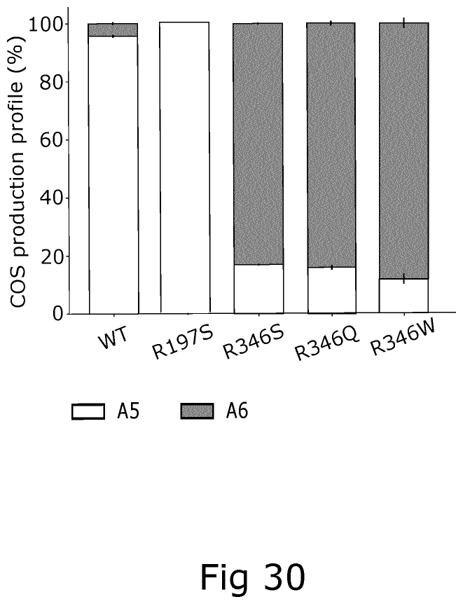

The positively charged amino acid arginine at amino acid position 197 (Arg-197) in RsNodC was substituted in to a neutral amino acid, i.e. serine (S), yielding mutation Arg-197-Ser (R197S). The positively charged amino acid arginine at amino acid position 346 (Arg-346) in RsNodC was substituted in to a neutral amino acid, i.e. serine (S), tryptophan (W), and glutamine (Q), yielding mutations Arg-346-Ser (R346S), Arg-346-Gln (R346Q) and Arg-346-Trp (R346W), respectively. All mutations were created in pCOSA5-RsNodC yielding plasmid pCOSA5-RsNodC R197S, pCOSA5-RsNodC R346S, pCOSA5-RsNodC R346Q, and pCOSA5-RsNodC R346W, respectively. Next, E. coli 3KO was transformed with pCOSA5-RsNodC (SEQ ID No 1), pCOSA5-RsNodC R197S (SEQ ID No 16), pCOSA5-RsNodC R346S (SEQ ID No 17), pCOSA5-RsNodC R346Q (SEQ ID No 18) and pCOSA5-RsNodC R346W (SEQ ID No 19) yielding E. coli sRsNodC1, E. coli sRsNodC14, E. coli sRsNodC15, E. coli sRsNodC16 and E. coli sRsNodC17, respectively. These metabolically engineered strains were grown in minimal medium with glucose. The chromatograms of COS production in E. coli sRsNodC1 and E. coli sRsNodC14-17 at 24 h are depicted in FIG. 7 and FIG. 26-29, respectively. The COS production profile E. coli sRsNodC1 and E. coli sRsNodC14-17 at 24 h are depicted in FIG. 30. Upon substituting a positively charged amino acid arginine present along one of the two transmembrane helices in RsNodC in to a negatively charged amino acid, the fraction of chitin oligosaccharides shifted towards chitin oligosaccharides with an increased and/or exclusive DP compared to the fraction of chitin oligosaccharides of the wild-type RsNodC. E. coli strains sRsNodC15, sRsNodC16 and sRsNodC17, carrying pCOSA5-RsNodC R346S, pCOSA5-RsNodC R346Q and pCOSA5-RsNodC R346W, respectively, depicted significant increased production of chitin oligosaccharides with a DP of six, regardless of the bulkiness of the neutral amino acid. E. coli strain sRsNodC14, carrying pCOSA5-RsNodC R197S, produced chitin oligosaccharides exclusively with a DP of five. sRsNodC1 produces fully acetylated chitintetraose, chitinpentaose, chitinhexaose and chitinheptaose with a ratio of 0/96/4/0. sRsNodC14 produces fully acetylated chitintetraose, chitinpentaose, chitinhexaose and chitinheptaose with a ratio of 0/100/0/0. sRsNodC15 produces fully acetylated chitintetraose, chitinpentaose, chitinhexaose and chitinheptaose with a ratio of 0/20/80/0. sRsNodC16 produces fully acetylated chitintetraose, chitinpentaose, chitinhexaose and chitinheptaose with a ratio of 0/20/80/0. sRsNodC17 produces fully acetylated chitintetraose, chitinpentaose, chitinhexaose and chitinheptaose with a ratio of 0/10/90/0.

### Example 10—Evaluation of a Substitution of an Arginine (R) Present Along One of the Two Transmembrane Helices in SmNodC in to a Neutral Amino Acid on COS Production

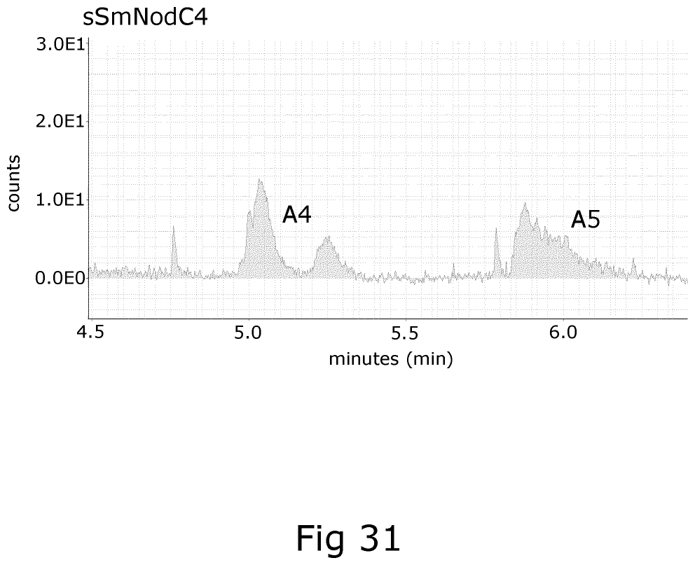

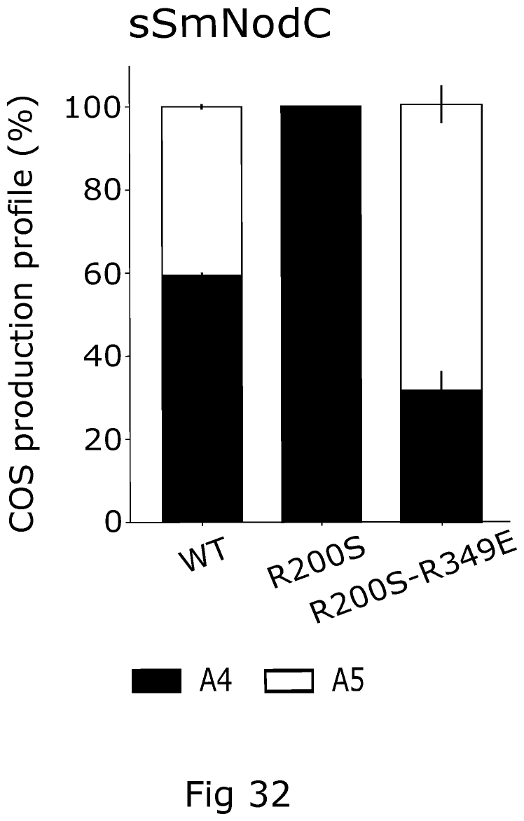

The positively charged amino acid arginine at amino acid position 200 (Arg-200) in SmNodC was substituted in to a neutral amino acid, i.e. serine (S), yielding mutation Arg-200-Ser (R200S). This mutation was created in pCOSA4-SmNodC yielding plasmid pCOSA4-SmNodC R200S. Next, E. coli 3KO was transformed with pCOSA4-SmNodC (SEQ ID No 2) and pCOSA4-SmNodC R200S (SEQ ID No 26), yielding E. coli sSmNodC1 and E. coli sSmNodC4, respectively. These metabolically engineered strains were grown in minimal medium with glucose. The chromatograms of COS production in E. coli sSmNodC1 and E. coli sSmNodC4 at 24 h are depicted in FIG. 3 and FIG. 31, respectively. The COS production profile of E. coli sSmNodC1 and E. coli sSmNodC4 at 24 h are depicted in FIG. 32. Upon substituting a positively charged amino acid arginine present along one of the two transmembrane helices in SmNodC in to a neutral amino acid, the fraction of chitin oligosaccharides significantly shifted towards chitin oligosaccharides with an exclusive DP compared to the fraction of chitin oligosaccharides of the wild-type SmNodC. E. coli sSmNodC4, carrying pCOSA4-SmNodC R200S, produced chitin oligosaccharides exclusively with a DP of four. sSmNodC1 produces fully acetylated chitintetraose, chitinpentaose, chitinhexaose and chitinheptaose with a ratio of 60/40/0/0. sSmNodC4 produces fully acetylated chitintetraose, chitinpentaose, chitinhexaose and chitinheptaose with a ratio of 100/0/0/0.

### Example 11—Evaluation of a Substitution of an Arginine (R) Present Along One of the Two Transmembrane Helices in RsNodC in to a Neutral Amino Acid Combined with an Additional Substitution of Another Positively Charged Amino Acid Present Along One of the Two Transmembrane Helices in RsNodC in to a Negatively Charged or Neutral Amino Acid on COS Production

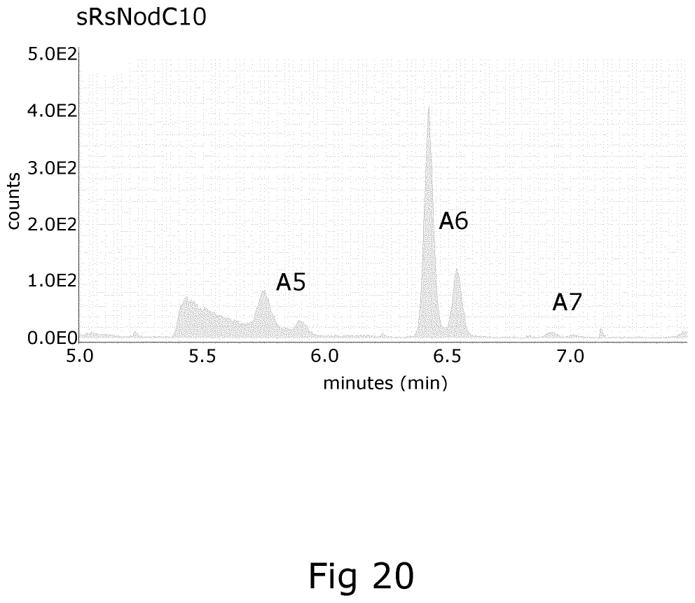

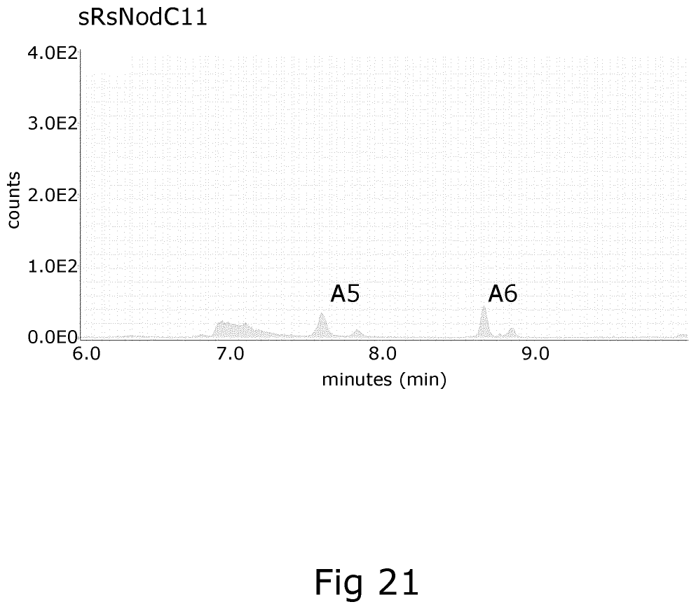

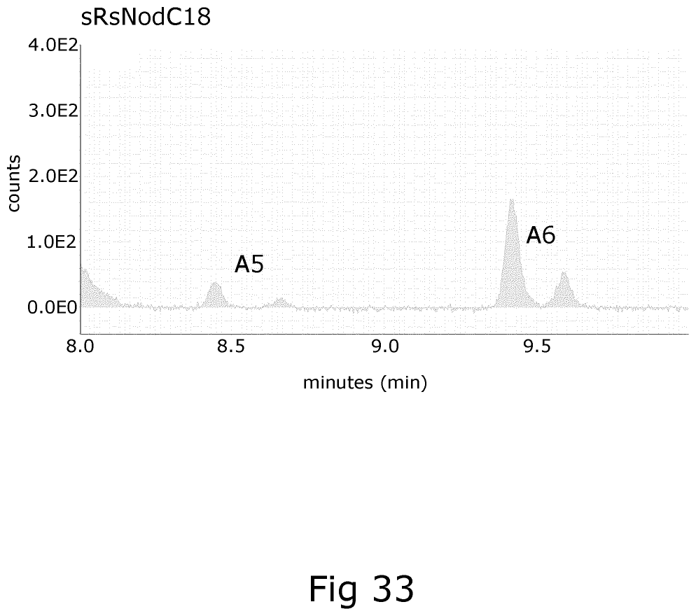

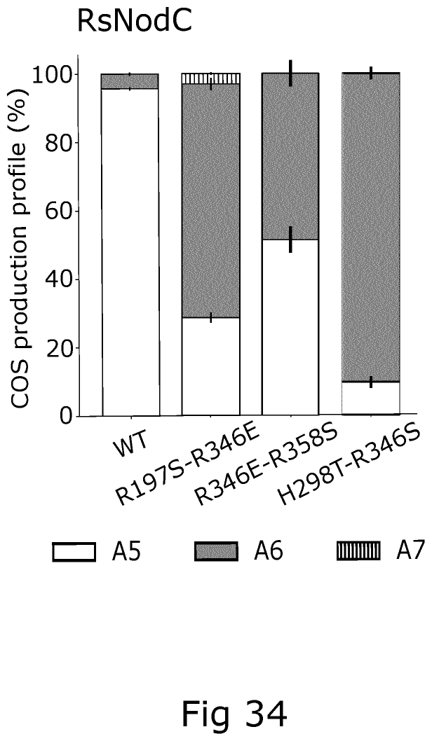

The positively charged amino acid arginine at amino acid position 197 (Arg-197) or amino acid position 346 (Arg-346) or at amino acid position 358 (Arg-358) in RsNodC was substituted in to a neutral amino acid, i.e. serine (S). Additionally, the positively charged amino acid histidine at amino acid position 298 (His-298) in RsNodC was substituted in to a neutral amino acid threonine (T), or the positively charged amino acid arginine at amino acid position 346 (Arg-346) in RsNodC was substituted in to a negatively charged amino acid glutamic acid (E), yielding the combined mutations His-298-Thr-Arg-346-Ser (H298T-R346S), Arg-197-Ser-Arg-346-Glu (R197S-R346E) and Arg-346-Glu-Arg-358-Ser (R346E-R358S), respectively. These mutations were created in pCOSA5-RsNodC yielding plasmid pCOSA5-RsNodC H298T-R346S, pCOSA5-RsNodC R197S-R346E and pCOSA5-RsNodC R346E-R358S. Next, E. coli 3KO was transformed with pCOSA5-RsNodC (SEQ ID No 1), pCOSA5-RsNodC H298T-R346S (SEQ ID No 20), pCOSA5-RsNodC R197S-R346E (SEQ ID No 12) and pCOSA5-RsNodC R346E-R358S (SEQ ID No 13) yielding E. coli sRsNodC1, E. coli sRsNodC18, E. coli sRsNodC10 and E. coli sRsNodC11, respectively. These metabolically engineered strains were grown in minimal medium with glucose. The chromatograms of COS production in E. coli sRsNodC1, E. coli sRsNodC18, E. coli sRsNodC10 and E. coli sRsNodC11 at 24 h are depicted in FIG. 7, FIG. 33, FIG. 20 and FIG. 21, respectively. The COS production profile of E. coli sRsNodC1, E. coli sRsNodC18, E. coli sRsNodC10 and E. coli sRsNodC11 at 24 h are depicted in FIG. 34. Upon combining a substitution of a positively charged amino acid arginine present along one of the two transmembrane helices in RsNodC in to a neutral amino acid with an additional substitution of another positively charged amino acid present along one of the two transmembrane helices in RsNodC in to a negatively charged or neutral amino acid, the fraction of the chitin oligosaccharides significantly shifted towards chitin oligosaccharides with an increased DP compared to the fraction of chitin oligosaccharides of the wild-type RsNodC. E. coli strains sRsNodC10, sRsNodC11 and sRsNodC18, carrying pCOSA5-RsNodC R197S-R346E, pCOSA5-RsNodC R346E-R358S, and pCOSA5-RsNodC H298T-R346S, respectively, depicted significant increased production of chitin oligosaccharides with a DP of six and/or seven. sRsNodC1 produces fully acetylated chitintetraose, chitinpentaose, chitinhexaose and chitinheptaose with a ratio of 0/96/4/0. sRsNodC10 produces fully acetylated chitintetraose, chitinpentaose, chitinhexaose and chitinheptaose with a ratio of 0/30/65/5. sRsNodC11 produces fully acetylated chitintetraose, chitinpentaose, chitinhexaose and chitinheptaose with a ratio of 0/50/50/0. sRsNodC18 produces fully acetylated chitintetraose, chitinpentaose, chitinhexaose and chitinheptaose with a ratio of 0/10/90/0.

### Example 12—Evaluation of a Substitution of an Arginine (R) Present Along One of the Two Transmembrane Helices in RsNodC in to a Neutral Amino Acid Combined with a Substitution of Methionine (M) Present Along One of the Two Transmembrane Helices in RsNodC in to a Serine (S), Alanine (A) or a Tryptophan (W) on COS Production

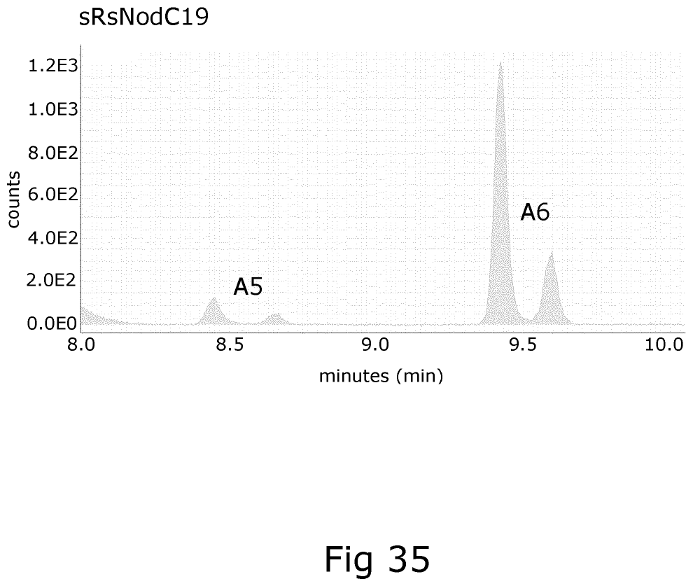

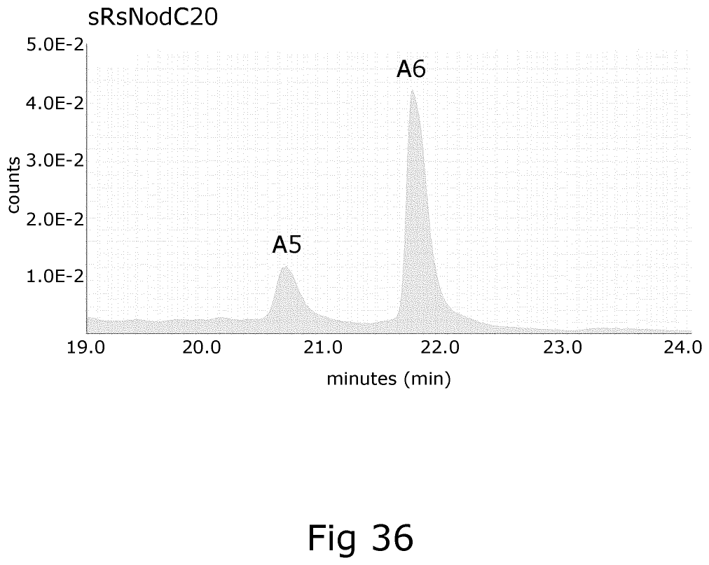

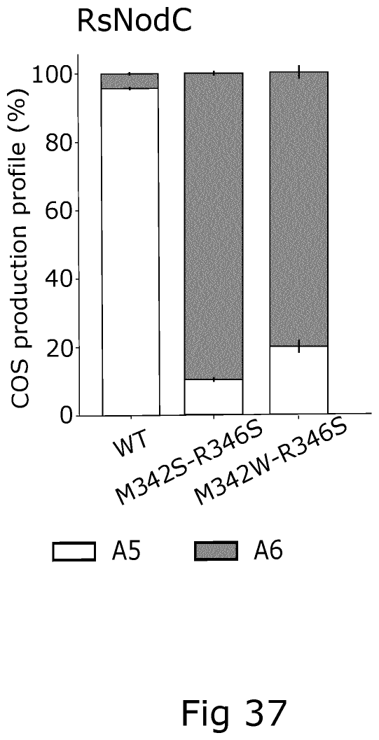

The positively charged amino acid arginine at amino acid position 346 (Arg-346) in RsNodC was substituted in to a neutral amino acid, i.e. serine (S). Additionally, the amino acid methionine at amino acid position 342 (Met-342) was substituted in to a neutral amino acid, i.e. serine (S) and tryptophan (W), yielding the combined mutations Met-342-Ser-Arg-346-Ser (M342S-R346S) and Met-342-Trp-Arg-346-Ser (M342W-R346S). These mutations were created in pCOSA5-RsNodC yielding plasmid pCOSA5-RsNodC M342S-R346S and pCOSA5-RsNodC M342W-R346S. Next, E. coli 3KO was transformed with pCOSA5-RsNodC (SEQ ID No 1), pCOSA5-RsNodC M342S-R346S (SEQ ID No 21) and pCOSA5-RsNodC M342W-R346S (SEQ ID No 22) yielding E. coli sRsNodC1, E. coli sRsNodC19 and E. coli sRsNodC20, respectively. These metabolically engineered strains were grown in minimal medium with glucose. The chromatograms of COS production in E. coli sRsNodC1 and E. coli sRsNodC19-20 at 24 h are depicted in FIG. 7 and FIG. 35 and FIG. 36, respectively. The COS production profile E. coli sRsNodC1 and E. coli sRsNodC19-20 at 24 h are depicted in FIG. 37. Upon combining a substitution of a positively charged amino acid arginine present along one of the two transmembrane helices in RsNodC in to a neutral amino acid with an additional substitution of a methionine present along one of the two transmembrane helices in RsNodC in to a neutral amino acid serine or tryptophan, the fraction of chitin significantly shifted towards chitin oligosaccharides with an increased DP compared to the fraction of chitin oligosaccharides of the wild-type RsNodC. E. coli strains sRsNodC19 and sRsNodC20, carrying pCOSA5-RsNodC M342S-R346S and pCOSA5-RsNodC M342W-R346S, respectively, depicted significant increased production of chitin oligosaccharides with a DP of six. sRsNodC1 produces fully acetylated chitintetraose, chitinpentaose, chitinhexaose and chitinheptaose with a ratio of 0/96/4/0. sRsNodC19 produces fully acetylated chitintetraose, chitinpentaose, chitinhexaose and chitinheptaose with a ratio of 0/10/90/0. sRsNodC20 produces fully acetylated chitintetraose, chitinpentaose, chitinhexaose and chitinheptaose with a ratio of 0/20/80/0.

### Example 13—Evaluation of a Substitution of a Histidine (H) Present Along One of the Two Transmembrane Helices in RsNodC in to a Neutral Amino Acid Combined with a Substitution of Leucine (L) Present Along One of the Two Transmembrane Helices in RsNodC in to a Threonine (T) on COS Production

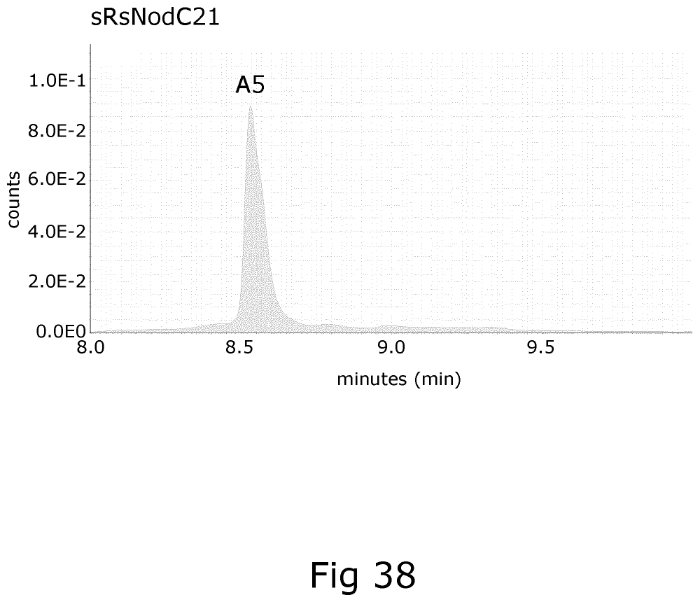

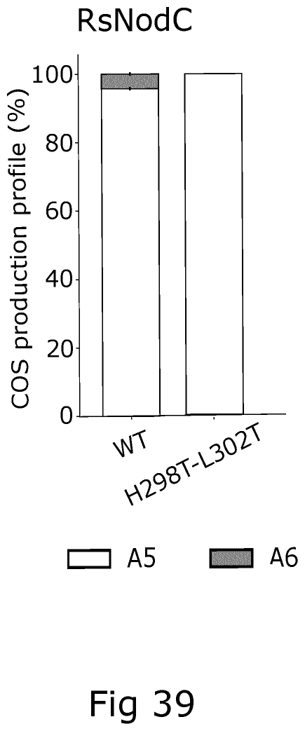

The positively charged amino acid histidine at amino acid position 298 (His-298) in RsNodC was substituted in to a neutral amino acid, i.e. threonine (T). Additionally, the amino acid leucine at amino acid position 302 (Leu-302) was substituted in to a neutral amino acid, i.e. threonine (T), yielding the combined mutations His-298-Thr-Leu-302-Thr (H298T-L302T). These mutations were created in pCOSA5-RsNodC yielding plasmid pCOSA5-RsNodC H298T-L302T. Next, E. coli 3KO was transformed with pCOSA5-RsNodC (SEQ ID No 1) and pCOSA5-RsNodC H298T-L302T (SEQ ID No 23) yielding E. coli sRsNodC1 and E. coli sRsNodC21, respectively. These metabolically engineered strains were grown in minimal medium with glucose. The chromatograms of COS production in E. coli sRsNodC1 and E. coli sRsNodC21 at 24 h are depicted in FIG. 7 and FIG. 38. The COS production profile E. coli sRsNodC1 and E. coli sRsNodC21 at 24 h are depicted in FIG. 39. Upon combining a substitution of a positively charged amino acid histidine present along one of the two transmembrane helices in RsNodC in to a neutral amino acid with an additional substitution of a leucine present along one of the two transmembrane helices in RsNodC in to a neutral amino acid threonine, the fraction of chitin oligosaccharides significantly shifted towards chitin oligosaccharides with an exclusive DP compared to the fraction of chitin oligosaccharides of the wild-type RsNodC. E. coli strains sRsNodC21, carrying pCOSA5-RsNodC H298T-L302T, produced chitin oligosaccharides exclusively with a DP of five. sRsNodC1 produces fully acetylated chitintetraose, chitinpentaose, chitinhexaose and chitinheptaose with a ratio of 0/96/4/0. sRsNodC21 produces fully acetylated chitintetraose, chitinpentaose, chitinhexaose and chitinheptaose with a ratio of 0/100/0/0.

### Example 14—Evaluation of a Substitution of an Arginine (R) Present Along One of the Two Transmembrane Helices in SmNodC in to a Negatively Charged Amino Acid Combined with an Additional Substitution of Another Positively Charged Amino Acid Present Along One of the Two Transmembrane Helices in SmNodC in to a Negatively Charged or Neutral Amino Acid on COS Production

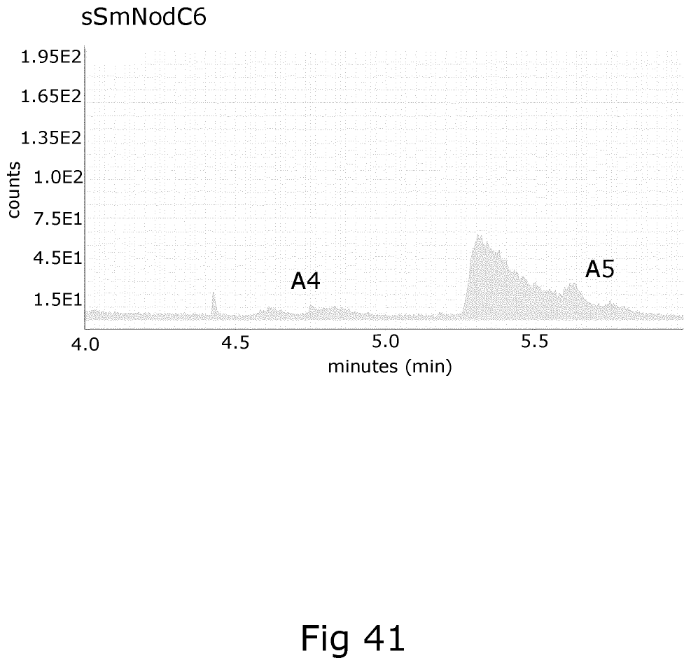

The positively charged amino acid arginine at amino acid position 200 (Arg-200) in SmNodC was substituted in to a neutral amino acid, i.e. serine (S). Additionally, the positively charged amino acid positively charged amino acid arginine at amino acid position 349 (Arg-349) in SmNodC was substituted in to a negatively charged amino acid glutamic acid (E), yielding the combined mutation Arg-200-Ser-Arg-349-Glu (R200S-R349E). These mutations were created in pCOSA4-SmNodC yielding plasmid pCOSA4-SmNodC R200S-R349E. Next, E. coli 3KO was transformed with pCOSA4-SmNodC (SEQ ID No 2) and pCOSA4-SmNodC R200S-R349E (SEQ ID No 27) yielding E. coli sSmNodC1 and E. coli sSmNodC6, respectively. These metabolically engineered strains were grown in minimal medium with glucose. The chromatograms of COS production in E. coli sSmNodC1 and E. coli sSmNodC6 at 24 h are depicted in FIG. 3 and FIG. 41, respectively. The COS production profile E. coli sSmNodC1 and E. coli sSmNodC6 at 24 h are depicted in FIG. 32. Upon combining a substitution of a positively charged amino acid arginine present along one of the two transmembrane helices in SmNodC in to a neutral amino acid with an additional substitution of another positively charged amino acid present along one of the two transmembrane helices in SmNodC in to a negatively charged or neutral amino acid, the fraction of the chitin oligosaccharides significantly shifted towards chitin oligosaccharides with an increased DP compared to the fraction of chitin oligosaccharides of the wild-type SmNodC. E. coli strain sSmNodC6 depicted significant increased production of chitin oligosaccharides with a DP of five. sSmNodC1 produces fully acetylated chitintetraose, chitinpentaose, chitinhexaose and chitinheptaose with a ratio of 60/40/0/0. sSmNodC6 produces fully acetylated chitintetraose, chitinpentaose, chitinhexaose and chitinheptaose with a ratio of 30/70/0/0.

### REFERENCES

Aam, B. B., E. B. Heggset, A. L. Norberg, M. So, and K. M. Vâ. 2010. “Production of Chitooligosaccharides and Their Potential Applications in Medicine.” Marine Drugs 8: 1482-1517.
Aerts, D., T. Verhaeghe, M. De Mey, T. Desmet, and W. Soetaert. 2011. “A Constitutive Expression System for High-Throughput Screening.” Engineering in Life Sciences 11 (1): 10-19.
Akhlaghi, S. P., R. C. Berry, and K. C. Tam. 2013. “Surface Modification of Cellulose Nanocrystal with Chitosan Oligosaccharide for Drug Delivery Applications.” Cellulose 20 (4): 1747-64.
Aranaz, I., N. Acosta, C. Civera, B. Elorza, J. Mingo, C. Castro, M. d. I. L. Gandia, and A. H. Caballero. 2018. “Cosmetics and Cosmeceutical Applications of Chitin, Chitosan and Their Derivatives.” Polymers 10 (2).
Basa, S., M. Nampally, T. Honorato, S. N. Das, A. R. Podile, N. E. El Gueddari, and B. M. Moerschbacher. 2020. “The Pattern of Acetylation Defines the Priming Activity of Chitosan Tetramers.” Journal of the American Chemical Society 142 (4): 1975-86.
Cabrera, J. C., and P. Van Cutsem. 2005. “Preparation of Chitooligosaccharides with Degree of Polymerization Higher than 6 by Acid or Enzymatic Degradation of Chitosan.” Biochemical Engineering Journal 25 (2): 165-72.
Datsenko, K. A., and B. L. Wanner. 2000. “One-Step Inactivation of Chromosomal Genes in Escherichia Coli K-12 Using PCR Products.” Proceedings of the National Academy of Sciences of the United States of America 97 (12): 6640-45.
Dorfmueller, H. C., A. T. Ferenbach, V. S. Borodkin, and D. M. F. Van Aalten. 2014. “A Structural and Biochemical Model of Processive Chitin Synthesis.” Journal of Biological Chemistry 289 (33): 23020-28.
Duan, X., G. Tian, D. Chen, J. Yang, L. Zhang, B. Li, L. Huang, et al. 2020. “Effects of Diet Chitosan Oligosaccharide on Performance and Immune Response of Sows and Their Offspring.” Livestock Science 239 (46).
Feng, F., J. Sun, G. V. Radhakrishnan, T. Lee, Z. Bozsóki, S. Fort, A. Gavrin, et al. 2019. “A Combination of Chitooligosaccharide and Lipochitooligosaccharide Recognition Promotes Arbuscular Mycorrhizal Associations in Medicago Truncatula.” Nature Communications 10 (1). Springer US.

Fliegmann, Judith, and Jean-jacques Bono. 2015. “Lipo-Chitooligosaccharidic Nodulation Factors and Their Perception by Plant Receptors.” Glycoconjugate Journal, 455-64.
Hayafune, M., R. Berisio, R. Marchetti, A. Silipo, M. Kayama, and Y. Desaki. 2014. “Chitin-Induced Activation of Immune Signaling by the Rice Receptor CEBiP Relies on a Unique Sandwich-Type Dimerization.” PNAS 111: E404-13.
Hedges, R., and A. Jacob. 1974. “Transposition of Ampicillin Resistance from RP4 to Other Replicon.” Molecular and General Genetics MGG 132: 31-40.
Kamst, E., and C. K. D. Breek. 2000. “Functional Analysis of Chimeras Derived from the Sinorhizobium Meliloti and Mesorhizobium Loti NodC Genes Identifies Regions Controlling Chitin Oligosaccharide Chain Length.” Molecular Genetics and Genomics 264:75-81.
Kaur, S., K. S. Dhugga, K. Gill, and J. Singh. 2016. “Novel Structural and Functional Motifs in Cellulose Synthase (CesA) Genes of Bread Wheat (Triticum Aestivum, L.).” PLOS ONE 11 (1): 1-18.
Knott, B. C., M. F. Crowley, M. E. Himmel, J. Zimmer, and G. T. Beckham. 2016. “Simulations of Cellulose Translocation in the Bacterial Cellulose Synthase Suggest a Regulatory Mechanism for the Dimeric Structure of Cellulose.” Chemical Science 7 (5): 3108-16.
McNamara, J. T., J. L. W. Morgan, and J. Zimmer. 2015. “A Molecular Description of Cellulose Biosynthesis.” Annual Review of Biochemistry 84 (1): 895-921.
Mey, M. De, J. Maertens, G. J. Lequeux, W. K. Soetaert, and E. J. Vandamme. 2007. “Construction and Model-Based Analysis of a Promoter Library for E. Coli: An Indispensable Tool for Metabolic Engineering.” BMC Biotechnology 7: 1-14.
Morgan, J. L. W., J. T. McNamara, and J. Zimmer. 2014. “Mechanism of Activation of Bacterial Cellulose Synthase by Cyclic-Di-GMP.” Nature Structural & Molecular Biology 21 (5): 489-96.
Morgan, J. L. W., J. Strumillo, and J. Zimmer. 2013. “Crystallographic Snapshot of Cellulose Synthesis and Membrane Translocation.” Nature 493 (7431): 181-86.
Oehme, D. P., T. Shafee, M. T. Downton, A. Bacic, and M. S. Doblin. 2019. “Differences in Protein Structural Regions That Impact Functional Specificity in GT2 Family β-Glucan Synthases.” PLOS ONE 14 (10): 1-26.
Ojeda-Hernandez, D. D., A. A. Canales-Aguirre, J. Matias-Guiu, U. Gomez-Pinedo, and J. C. Mateos-Diaz. 2020. “Potential of Chitosan and Its Derivatives for Biomedical Applications in the Central Nervous System.” Frontiers in Bioengineering and Biotechnology 8 (May): 1-15.
Ouyang, Q. Q., S. Zhao, S. D. Li, and C. Song. 2017. “Application of Chitosan, Chitooligosaccharide, and Their Derivatives in the Treatment of Alzheimer's Disease.” Marine Drugs 15 (11): 1-15.
Perez-Mendoza, D., D. Bertinetti, R. Lorenz, M. T. Gallegos, F. W. Herberg, and J. Sanjuan. 2017. “A Novel C-Di-GMP Binding Domain in Glycosyltransferase BgsA Is Responsible for the Synthesis of a Mixed-Linkage β-Glucan.” Scientific Reports 7 (1): 1-11.
Prentki, Pierre, and Henry M Krisch. 1982. “A Modified PBR322 Vector with Improved Properties for the Cloning, Recovery, and Sequencing of Blunt-Ended DNA Fragments.” Gene 17: 189-96.
Quan, J., and J. Tian. 2009. “Circular Polymerase Extension Cloning of Complex Gene Libraries and Pathways.” Plos One 4 (7): e6441.
Rakkhumkaew, N., and C. Pengsuk. 2018. “Chitosan and Chitooligosaccharides from Shrimp Shell Waste: Characterization, Antimicrobial and Shelf Life Extension in Bread.” Food Science and Biotechnology 27 (4). Springer Singapore: 1201-8.
Rao, M. S., R. Chander, and A. Sharma. 2008. “Synergistic Effect of Chitooligosaccharides and Lysozyme for Meat Preservation.” LWT—Food Science and Technology 41 (10): 1995-2001.
Sambrook, J. F., and D. W. Russell. 2001. Molecular Cloning: A Laboratory Manual. 3rd ed. Cold Spring Harbor Laboratory Press.
Shcherbo, Dmitry, Christopher S Murphy, Galina V Ermakova, Elena A Solovieva, Tatiana V Chepurnykh, Aleksandr S Shcheglov, V Vladislav, et al. 2009. “Far-Red Fluorescent Tags for Protein Imaging in Living Tissues.” Biochemical Journal 418 (3): 567-74. doi:10.1042/BJ20081949.Far-red.
Wan, J., F. Jiang, Q. Xu, D. Chen, B. Yu, Z. Huang, X. Mao, J. Yu, and J. He. 2017. “New Insights into the Role of Chitosan Oligosaccharide in Enhancing Growth Performance, Antioxidant Capacity, Immunity and Intestinal Development of Weaned Pigs.” RSC Advances 7 (16): 9669-79.
Wan, J., X. C. Zhang, D. Neece, K. M. Ramonell, S. Clough, S. Y. Kim, M. G. Stacey, and G. Stacey. 2008. “A LysM Receptor-like Kinase Plays a Critical Role in Chitin Signaling and Fungal Resistance in Arabidopsis.” Plant Cell 20 (2): 471-81.
Wolinsky, J. B., Y. L. Colson, and M. W. Grinstaff. 2012. “Local Drug Delivery Strategies for Cancer Treatment: Gels, Nanoparticles, Polymeric Films, Rods, and Wafers.” Journal of Controlled Release 159 (1): 3333. doi:10.1016/j.jconrel.2011.11.031.Local.
Xu, X., L. Sun, L. Zhou, Y. Cheng, and F. Cao. 2020. “Functional Chitosan Oligosaccharide Nanomicelles for Topical Ocular Drug Delivery of Dexamethasone.” Carbohydrate Polymers 227 (June 2019). Elsevier: 115356.
Yang, G., and Y. Ding. 2014. “Recent Advances in Biocatalyst Discovery, Development and Applications.” Bioorganic and Medicinal Chemistry 22 (20): 5604-12.
Yu, S., X. Xu, J. Feng, M. Liu, and K. Hu. 2019. “Chitosan and Chitosan Coating Nanoparticles for the Treatment of Brain Disease.” International Journal of Pharmaceutics 560 (January): 282-93.

1. Use of a mutated, bacterial chitin oligosaccharide synthase to increase at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%, and, wherein said mutation is a substitution of at least one charged amino acid present in one of the two transmembrane helices of said chitin oligosaccharide synthase by an amino acid having the opposite charge or by an amino acid having no charge.

2. Use of a mutated, bacterial chitin oligosaccharide synthase according to claim 1 wherein said oligosaccharide synthase is from a bacterial species belonging to the genus Rhizobium, Sinorhizobium, Bosea, Bradyrhizobium, Ochrobactrum, Devosia, Aminobacter, Mesorhizobium, Phyllobacterium, Agrobacterium, Allorhizobium, Neorhizobium, Shinella, Azorhizobium, Paraburkholderia and Pseudomonas Cupriavidus, Burkholderia, Corallococcus, Desulfobacterium, Actinobacteria, Methylobacteria, Microvirga or Brucella.

3. Use of a mutated, bacterial chitin oligosaccharide synthase according to claims 1-2 wherein said transmembrane helices have an amino acid sequence identity of 30 to 100% to transmembrane helices of enzymes chosen from the group of: beta-polysaccharide synthase, chitin synthase, chitooligosaccharide synthase, N-acetylglucosaminyltransferase, beta-1,4-N-acetylglucosaminyltransferase, cellulose synthase, hyaluronan synthase, glycosyl transferase family 2, hyaluronic acid synthase, Nodulation protein C or NodC-like enzyme.

4. A mutated, bacterial chitin oligosaccharide synthase, wherein said mutation is a substitution of at least one charged amino acid present in one of the two transmembrane helices of said chitin oligosaccharide synthase by an amino acid having the opposite charge or by an amino acid having no charge, wherein said transmembrane helix comprises the amino acid regions 187-200 and 295-370 of SEQ ID No 1, 190-203 and 298-373 of SEQ ID No 2, or, 190-200 and 298-373 of SEQ ID No 3, and, wherein said substitution is not an arginine which is substituted by a serine at amino acid position 349 of the chitin oligosaccharide synthase consisting of the amino acid sequence given by SEQ ID No 2.

5. A mutated chitin oligosaccharide synthase according to claim 4 wherein said charged amino acid is chosen from the list consisting of arginine, histidine and lysine, and, wherein said amino acid having the opposite charge is aspartic acid or glutamic acid.

6. A mutated chitin oligosaccharide synthase according to claim 5 wherein said charged amino acid is an arginine at position 197, 346, 353, 355 or 358 of SEQ ID No 1.

7. A mutated chitin oligosaccharide synthase according to claim 6 wherein said charged amino acid is an arginine at position 346 of SEQ ID No 1 and wherein an arginine at amino acid position 197, 353, 355 or 358 of SEQ ID No 1 is substituted by glutamic acid or serine.

8. A mutated chitin oligosaccharide synthase according to claim 6 wherein a methionine at amino acid position 342 of SEQ ID No 1 is substituted by serine, tryptophan or alanine.

9. A mutated chitin oligosaccharide synthase according to claim 6 wherein a histidine at amino acid position 298 of SEQ ID No 1 is substituted by threonine.

10. A mutated chitin oligosaccharide synthase according to claim 5 wherein said charged amino acid is a histidine at position 298 in SEQ ID No 1 and wherein said amino acid having the opposite charge is aspartic acid or glutamic acid.

11. A mutated chitin oligosaccharide synthase according to claim 5 wherein said charged amino acid is an arginine at position 200 or 349 in SEQ ID No 2 and wherein said amino acid having the opposite charge is aspartic acid or glutamic acid.

12. A mutated chitin oligosaccharide synthase according to claim 11 wherein said charged amino acid is an arginine at amino acid position 349 of SEQ ID No 2, wherein said amino acid having the opposite charge is aspartic acid or glutamic acid, and wherein an arginine at amino acid position 200 of SEQ ID No 2 is substituted by serine.

13. A mutated chitin oligosaccharide synthase according to claim 5 wherein said charged amino acid is an arginine at position 349 of SEQ ID No 3, and, wherein said amino acid having the opposite charge is aspartic acid or glutamic acid.

14. A mutated chitin oligosaccharide synthase according to claim 4 wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan.

15. A mutated chitin oligosaccharide synthase according to claim 4 wherein said charged amino acid is chosen from the list consisting of an arginine, histidine and lysine and wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan.

16. A mutated chitin oligosaccharide synthase according to claim 15 wherein said charged amino acid is an arginine or histidine within the amino acid regions 187-200 and 295-370 of SEQ ID No 1, 190-203 and 298-373 of SEQ ID No 2, or, 190-200 and 298-373 of SEQ ID No 3 and wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine or tryptophan.

17. A mutated chitin oligosaccharide synthase according according to claim 16 wherein said charged amino acid is an arginine at position 197, 346, 353, 355 or 358 in SEQ ID No 1 or a histidine at position 298 in SEQ ID No 1 and wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan.

18. A mutated chitin oligosaccharide synthase according to claim 17 wherein said charged amino acid is an arginine at amino acid position 346 of SEQ ID No 1, wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan, and wherein a methionine at amino acid position 342 of SEQ ID No 1 is substituted by serine, tryptophan, or alanine.

19. A mutated chitin oligosaccharide synthase according to claim 17 wherein said charged amino acid is an arginine at amino acid position 346 of SEQ ID No 1, wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan and wherein a histidine at amino acid position 298 of SEQ ID No 1 is substituted by threonine.

20. A mutated chitin oligosaccharide synthase according to claim 16 wherein said charged amino acid is an histidine at amino acid position 298 of SEQ ID No 1, wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan, and wherein a leucine at amino acid position 302 of SEQ ID No 1 is substituted by threonine.

21. A mutated chitin oligosaccharide synthase according to claim 16 wherein said charged amino acid is an arginine at position 200 or 349 of SEQ ID No 2 or at position 200 or 349 of SEQ ID No 3, and, wherein said amino acid having no charge is chosen from the list consisting of serine, threonine, asparagine, glutamine, cysteine, selenocysteine, glycine, proline, alanine, valine, isoleucine, leucine, methionine, phenylalanine, tyrosine and tryptophan.

22. Use of a mutated, bacterial chitin oligosaccharide synthase according to claims 4-21 to increase at least one of the percentages of the fractions of the chitin oligosaccharides having a degree of polymerization equal to 4, 5, 6 or 7 when compared to the percentages of the fractions of the chitin oligosaccharides having a degree of polymerization equal to 4, 5, 6 or 7 as produced by the corresponding wild type chitin oligosaccharide synthase, wherein the sum of the latter percentages of said fractions is 100%.


SEQUENCE LISTING (100 sequences)

SEQ ID NO: 1 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASMTMIRCAVAAVRARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 2 - protein, 438 aa, synthetic construct
MYLLDTTSTAAISIYALLLTAYRSMQVLHARPIDGPAVSAEPVETRPLPAVDVIVPSFNEDPGILSACLASIADQDYPGELRVYVVDDGSRNREAILRVRAFYSRDPRFSFILLPENVGKRKAQIAAIGQSSGDLVLNVDSDSTIAFDVVSKLALKMRDPEVGAVMGQLTASNSGDTWLTKLIDMEYWLACNEERAAQSRFGAVMCCCGPCAMYRRSALASLLDQYETQLFRGKLSDFGEDRHLTILMLKAGFRTEYVPNAIVATVVPDTLKSYLRQQLRWARSTFRDTFLALPLLRGLNPFLTFDVVGQNIGPLLLALSVVTGLAHFITTATVPWWTILIIACMTIIRCSVVALHARQLRFLGFVLHTPINLFLLLPLKAYALCTLSNSDWLSRYSAPEVPVSGGKPTPIQASGRVTPDCTCSGELRRQWSHPQFEK

SEQ ID NO: 3 - protein, 413 aa, synthetic construct
MDLLGTTGAVAISLYAALSTAYKGMQAIYALPTNTTAASTPVTGSGAPPSVDVIVPCYNEDPRALSACLASIAKQDYAGELRVYVVDDGSGNRNAIIPVHDHYACDPRFRFILMPKNVGKRKAQIVAIRESSGDLVLNVDSDTTIAPDVVTKLALKMYSPAVGAAMGQLTASNRSDTWLTRLIDMEYWLACNEERAAQARFGAVMCCCGPCAMYRRSALLLLLDKYETQLFRGRPSDFGEDRHLTILMLNAGFRTEYVPDAIAATVVPNSMGAYLRQQLRWARSTFRDTLLALRLLPGLDRYLTLDVIGQNLGPLLLALSVLTGLAQLALTATVPWSTILMIASMTMVRCGVAAFRARELRFLGFSLHTLLNVALLLPLKAYALCTLSNSDWLSRGSPAAAPNGVKDSPEPHC

SEQ ID NO: 4 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASMTMIDCAVAAVRARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 5 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASMTMIECAVAAVRARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 6 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASMTMIRCAVAAVEARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 7 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASMTMIRCAVAAVRAEQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 8 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASMTMIRCAVAAVRARQLEFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 9 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASMTMIECAVAAVEARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 10 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASMTMIECAVAAVRAEQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 11 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASMTMIECAVAAVRARQLEFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 12 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQASFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASMTMIECAVAAVRARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 13 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASMTMIECAVAAVRARQLSFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 14 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDTYITLDVIGQNLGPLLLALAVLTGVLQVALTATVPLWTVMMIASMTMIECAVAAVRARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 15 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASSTMIECAVAAVRARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 16 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQASFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASMTMIRCAVAAVRARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 17 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASMTMISCAVAAVRARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 18 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASMTMIQCAVAAVRARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 19 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASMTMIWCAVAAVRARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 20 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDTYITLDVIGQNLGPLLLALAVLTGVLQVALTATVPLWTVMMIASMTMISCAVAAVRARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 21 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASSTMISCAVAAVRARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 22 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDHYITLDVIGQNLGPLLLALAVLTGVLQVALTATVPLWTVMMIASWTMISCAVAAVRARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 23 - protein, 443 aa, synthetic construct
MDLLNTIGIGAVSCYALLSTAHKSMQTLYAQPKDQSSASEDFAFLPSVDIIVPCYNENPHTFSECLASIANQDYAGKLRVYVVDDGSANREKLERVHHTYAGDPRFDFILLRENVGKRKAQIAAIRGSSGDLVLNVDSDSTLASDVVTKLALKMQNPEIGAAMGQLTASNRNDTWLTRLIDMEYWLACNEERAAQARFGAVMCCCGPCAMYRRSALLSLLDQYESQFFRGKPSDFGEDRHLTILMLKAGFRTDYVPDAIAATVVPDRMGPYLRQQLRWARSTFRDTLLALRLLPGLDTYITTDVIGQNLGPLLLALAVLTGVLQVALTATVPLWTVMMIASMTMIRCAVAAVRARQLRFLVFSLHTPINLFFLLPMKAYALCTLSNSDWLSRSSPANKTSAGGEHPTTEASAGGTSGNATPLRRLNLARDSSTVTPAGVYSDD

SEQ ID NO: 24 - protein, 278 aa, synthetic construct
EVGAVMGQLTASNSGDTWLTKLIDMEYWLACNEERAAQSRFGAVMCCCGPCAMYRRSALASLLDQYETQLFRGKLSDFGEDRHLTILMLKAGFRTEYVPNAIVATVVPDTLKSYLRQQLRWARSTFRDTFLALPLLRGLNPFLTFDVVGQNIGPLLLALSVVTGLAHFITTATVPWWTILIIACMTIISCSVVALHARQLRFLGFVLHTPINLFLLLPLKAYALCTLSNSDWLSRYSAPEVPVSGGKPTPIQASGRVTPDCTCSGELRRQWSHPQFEK

SEQ ID NO: 25 - protein, 438 aa, synthetic construct
MYLLDTTSTAAISIYALLLTAYRSMQVLHARPIDGPAVSAEPVETRPLPAVDVIVPSFNEDPGILSACLASIADQDYPGELRVYVVDDGSRNREAILRVRAFYSRDPRFSFILLPENVGKRKAQIAAIGQSSGDLVLNVDSDSTIAFDVVSKLALKMRDPEVGAVMGQLTASNSGDTWLTKLIDMEYWLACNEERAAQSRFGAVMCCCGPCAMYRRSALASLLDQYETQLFRGKLSDFGEDRHLTILMLKAGFRTEYVPNAIVATVVPDTLKSYLRQQLRWARSTFRDTFLALPLLRGLNPFLTFDVVGQNIGPLLLALSVVTGLAHFITTATVPWWTILIIACMTIIECSVVALHARQLRFLGFVLHTPINLFLLLPLKAYALCTLSNSDWLSRYSAPEVPVSGGKPTPIQASGRVTPDCTCSGELRRQWSHPQFEK

SEQ ID NO: 26 - protein, 438 aa, synthetic construct
MYLLDTTSTAAISIYALLLTAYRSMQVLHARPIDGPAVSAEPVETRPLPAVDVIVPSFNEDPGILSACLASIADQDYPGELRVYVVDDGSRNREAILRVRAFYSRDPRFSFILLPENVGKRKAQIAAIGQSSGDLVLNVDSDSTIAFDVVSKLALKMRDPEVGAVMGQLTASNSGDTWLTKLIDMEYWLACNEERAAQSSFGAVMCCCGPCAMYRRSALASLLDQYETQLFRGKLSDFGEDRHLTILMLKAGFRTEYVPNAIVATVVPDTLKSYLRQQLRWARSTFRDTFLALPLLRGLNPFLTFDVVGQNIGPLLLALSVVTGLAHFITTATVPWWTILIIACMTIIRCSVVALHARQLRFLGFVLHTPINLFLLLPLKAYALCTLSNSDWLSRYSAPEVPVSGGKPTPIQASGRVTPDCTCSGELRRQWSHPQFEK

SEQ ID NO: 27 - protein, 438 aa, synthetic construct
MYLLDTTSTAAISIYALLLTAYRSMQVLHARPIDGPAVSAEPVETRPLPAVDVIVPSFNEDPGILSACLASIADQDYPGELRVYVVDDGSRNREAILRVRAFYSRDPRFSFILLPENVGKRKAQIAAIGQSSGDLVLNVDSDSTIAFDVVSKLALKMRDPEVGAVMGQLTASNSGDTWLTKLIDMEYWLACNEERAAQSSFGAVMCCCGPCAMYRRSALASLLDQYETQLFRGKLSDFGEDRHLTILMLKAGFRTEYVPNAIVATVVPDTLKSYLRQQLRWARSTFRDTFLALPLLRGLNPFLTFDVVGQNIGPLLLALSVVTGLAHFITTATVPWWTILIIACMTIIECSVVALHARQLRFLGFVLHTPINLFLLLPLKAYALCTLSNSDWLSRYSAPEVPVSGGKPTPIQASGRVTPDCTCSGELRRQWSHPQFEK

SEQ ID NO: 28 - protein, 413 aa, synthetic construct
MDLLGTTGAVAISLYAALSTAYKGMQAIYALPTNTTAASTPVTGSGAPPSVDVIVPCYNEDPRALSACLASIAKQDYAGELRVYVVDDGSGNRNAIIPVHDHYACDPRFRFILMPKNVGKRKAQIVAIRESSGDLVLNVDSDTTIAPDVVTKLALKMYSPAVGAAMGQLTASNRSDTWLTRLIDMEYWLACNEERAAQARFGAVMCCCGPCAMYRRSALLLLLDKYETQLFRGRPSDFGEDRHLTILMLNAGFRTEYVPDAIAATVVPNSMGAYLRQQLRWARSTFRDTLLALRLLPGLDRYLTLDVIGQNLGPLLLALSVLTGLAQLALTATVPWSTILMIASMTMVECGVAAFRARELRFLGFSLHTLLNVALLLPLKAYALCTLSNSDWLSRGSPAAAPNGVKDSPEPHC

SEQ ID NO: 29 - protein, 413 aa, synthetic construct
MDLLGTTGAVAISLYAALSTAYKGMQAIYALPTNTTAASTPVTGSGAPPSVDVIVPCYNEDPRALSACLASIAKQDYAGELRVYVVDDGSGNRNAIIPVHDHYACDPRFRFILMPKNVGKRKAQIVAIRESSGDLVLNVDSDTTIAPDVVTKLALKMYSPAVGAAMGQLTASNRSDTWLTRLIDMEYWLACNEERAAQARFGAVMCCCGPCAMYRRSALLLLLDKYETQLFRGRPSDFGEDRHLTILMLNAGFRTEYVPDAIAATVVPNSMGAYLRQQLRWARSTFRDTLLALRLLPGLDRYLTLDVIGQNLGPLLLALSVLTGLAQLALTATVPWSTILMIASMTMVSCGVAAFRARELRFLGFSLHTLLNVALLLPLKAYALCTLSNSDWLSRGSPAAAPNGVKDSPEPHC

SEQ ID NO: 30 - protein, 438 aa, synthetic construct
MYLLDTTSTAAISIYALLLTAYRSMQVLHARPIDGPAVSAEPVETRPLPAVDVIVPSFNEDPGILSACLASIADQDYPGELRVYVVDDGSRNREAILRVRAFYSRDPRFSFILLPENVGKRKAQIAAIGQSSGDLVLNVDSDSTIAFDVVSKLALKMRDPEVGAVMGQLTASNSGDTWLTKLIDMEYWLACNEERAAQSRFGAVMCCCGPCAMYRRSALASLLDQYETQLFRGKLSDFGEDRHLTILMLKAGFRTEYVPNAIVATVVPDTLKSYLRQQLRWARSTFRDTFLALPLLRGLNPFLTFDVVGQNIGPLLLALSVVTGLAHFITTATVPWWTILIIACMTIIRCSVVALRARQLRFLGFVLHTPINLFLLLPLKAYALCTLSNSDWLSRYSAPEVPVSGGKPTPIQASGRVTPDCTCSGELRRQWSHPQFEK

SEQ ID NO: 31 - DNA, 72 bp, synthetic construct
  misc_feature 1..72: note = Primer
  source 1..72: mol_type = other DNA
ggaattaatcgccggatgcaaggttcacgccgcatctggcaaacatcctcacgtgtaggctggagctgcttc

SEQ ID NO: 32 - DNA, 69 bp, synthetic construct
  misc_feature 1..69: note = Primer
  source 1..69: mol_type = other DNA
ggcttgcggagtgtctggctgacagataatcgtcgatgagggcagttttcatatgaatatcctccttag

SEQ ID NO: 33 - DNA, 21 bp, synthetic construct
  misc_feature 1..21: note = Primer
  source 1..21: mol_type = other DNA
tattcccatccgcgtctgttc

SEQ ID NO: 34 - DNA, 21 bp, synthetic construct
  misc_feature 1..21: note = Primer
  source 1..21: mol_type = other DNA
aagcgcccaatgtattccagg

SEQ ID NO: 35 - DNA, 75 bp, synthetic construct
  misc_feature 1..75: note = Primer
  source 1..75: mol_type = other DNA
ggctggccgatgacacctggcggcagctattaataaaacaataaggagagcagtcagcattacacgtcttgagcg

SEQ ID NO: 36 - DNA, 75 bp, synthetic construct
  misc_feature 1..75: note = Primer
  source 1..75: mol_type = other DNA
ctgattcagacgggtgctgatcgctttccagcgagccgagtccatcagttcctgccatatgaatatcctccttag

SEQ ID NO: 37 - DNA, 20 bp, synthetic construct
  misc_feature 1..20: note = Primer
  source 1..20: mol_type = other DNA
cggcgcaattatggcgtcag

SEQ ID NO: 38 - DNA, 21 bp, synthetic construct
  misc_feature 1..21: note = Primer
  source 1..21: mol_type = other DNA
cggactgttagagtcaaaacc

SEQ ID NO: 39 - DNA, 67 bp, synthetic construct
  misc_feature 1..67: note = Primer
  source 1..67: mol_type = other DNA
taattcctgcgtaggacttttgttttgcagtttttacgtcacaagggcatatgaatatcctccttag

SEQ ID NO: 40 - DNA, 75 bp, synthetic construct
  misc_feature 1..75: note = Primer
  source 1..75: mol_type = other DNA
gtagcccattgacaaaaaatgcggcgatactggaaggtatcgccaacacgtgtaggctggagctggagctgcttc

SEQ ID NO: 41 - DNA, 22 bp, synthetic construct
  misc_feature 1..22: note = Primer
  source 1..22: mol_type = other DNA
gagactcccgtatactttcttc

SEQ ID NO: 42 - DNA, 20 bp, synthetic construct
  misc_feature 1..20: note = Primer
  source 1..20: mol_type = other DNA
cgccctttttgcatttgttg

SEQ ID NO: 43 - DNA, 43 bp, synthetic construct
  misc_feature 1..43: note = Primer
  source 1..43: mol_type = other DNA
gcgtgcatgaccattataagctgcagcgtcgtagcattgcatg

SEQ ID NO: 44 - DNA, 43 bp, synthetic construct
  misc_feature 1..43: note = Primer
  source 1..43: mol_type = other DNA
catgcaatgctacgacgctgcagcttataatggtcatgcacgc

SEQ ID NO: 45 - DNA, 46 bp, synthetic construct
  misc_feature 1..46: note = Primer
  source 1..46: mol_type = other DNA
ccattatagaatgcagcgtcgtagcattgcatgctcgccaacttag

SEQ ID NO: 46 - DNA, 29 bp, synthetic construct
  misc_feature 1..29: note = Primer
  source 1..29: mol_type = other DNA
gacgctgcattctataatggtcatgcacg

SEQ ID NO: 47 - DNA, 33 bp, synthetic construct
  misc_feature 1..33: note = Primer
  source 1..33: mol_type = other DNA
ggcacagtctagcttcggtgctgttatgtgttg

SEQ ID NO: 48 - DNA, 20 bp, synthetic construct
  misc_feature 1..20: note = Primer
  source 1..20: mol_type = other DNA
caccgaagctagactgtgcc

SEQ ID NO: 49 - DNA, 32 bp, synthetic construct
  misc_feature 1..32: note = Primer
  source 1..32: mol_type = other DNA
gacgatgatcgattgtgcagttgcagcagtcc

SEQ ID NO: 50 - DNA, 40 bp, synthetic construct
  misc_feature 1..40: note = Primer
  source 1..40: mol_type = other DNA
ctgcacaatcgatcatcgtcattgatgcaatcatcatcac

SEQ ID NO: 51 - DNA, 25 bp, synthetic construct
  misc_feature 1..25: note = Primer
  source 1..25: mol_type = other DNA
cgatgatcgaatgtgcagttgcagc

SEQ ID NO: 52 - DNA, 24 bp, synthetic construct
  misc_feature 1..24: note = Primer
  source 1..24: mol_type = other DNA
gctgcaactgcacattcgatcatc

SEQ ID NO: 53 - DNA, 23 bp, synthetic construct
  misc_feature 1..23: note = Primer
  source 1..23: mol_type = other DNA
ttgcagcagtcgaagcacgtcag

SEQ ID NO: 54 - DNA, 26 bp, synthetic construct
  misc_feature 1..26: note = Primer
  source 1..26: mol_type = other DNA
ctgacgtgcttcgactgctgcaactg

SEQ ID NO: 55 - DNA, 26 bp, synthetic construct
  misc_feature 1..26: note = Primer
  source 1..26: mol_type = other DNA
ccgtgcagaacagctgcgctttctgg

SEQ ID NO: 56 - DNA, 33 bp, synthetic construct
  misc_feature 1..33: note = Primer
  source 1..33: mol_type = other DNA
agcgcagctgttctgcacggactgctgcaactg

SEQ ID NO: 57 - DNA, 22 bp, synthetic construct
  misc_feature 1..22: note = Primer
  source 1..22: mol_type = other DNA
gcacgtcagctggaatttctgg

SEQ ID NO: 58 - DNA, 21 bp, synthetic construct
  misc_feature 1..21: note = Primer
  source 1..21: mol_type = other DNA
ccagaaattccagctgacgtg

SEQ ID NO: 59 - DNA, 23 bp, synthetic construct
  misc_feature 1..23: note = Primer
  source 1..23: mol_type = other DNA
ttgcagcagtcgaagcacgtcag

SEQ ID NO: 60 - DNA, 26 bp, synthetic construct
  misc_feature 1..26: note = Primer
  source 1..26: mol_type = other DNA
ctgacgtgcttcgactgctgcaactg

SEQ ID NO: 61 - DNA, 26 bp, synthetic construct
  misc_feature 1..26: note = Primer
  source 1..26: mol_type = other DNA
ccgtgcagaacagctgcgctttctgg

SEQ ID NO: 62 - DNA, 33 bp, synthetic construct; identical to SEQ ID NO: 56
  misc_feature 1..33: note = Primer
  source 1..33: mol_type = other DNA

SEQ ID NO: 63 - DNA, 22 bp, synthetic construct
  misc_feature 1..22: note = Primer
  source 1..22: mol_type = other DNA
gcacgtcagctggaatttctgg

SEQ ID NO: 64 - DNA, 21 bp, synthetic construct
  misc_feature 1..21: note = Primer
  source 1..21: mol_type = other DNA
ccagaaattccagctgacgtg

SEQ ID NO: 65 - DNA, 27 bp, synthetic construct
  misc_feature 1..27: note = Primer
  source 1..27: mol_type = other DNA
gcacaggcaagctttggtgcagtgatg

SEQ ID NO: 66 - DNA, 22 bp, synthetic construct
  misc_feature 1..22: note = Primer
  source 1..22: mol_type = other DNA
gcaccaaagcttgcctgtgctg

SEQ ID NO: 67 - DNA, 22 bp, synthetic construct
  misc_feature 1..22: note = Primer
  source 1..22: mol_type = other DNA
gcacgtcagctgagctttctgg

SEQ ID NO: 68 - DNA, 30 bp, synthetic construct
  misc_feature 1..30: note = Primer
  source 1..30: mol_type = other DNA
ccagaaagctcagctgacgtgcacggactg

SEQ ID NO: 69 - DNA, 24 bp, synthetic construct
  misc_feature 1..24: note = Primer
  source 1..24: mol_type = other DNA
gccgggtctggatacctatattac

SEQ ID NO: 70 - DNA, 29 bp, synthetic construct
  misc_feature 1..29: note = Primer
  source 1..29: mol_type = other DNA
atataggtatccagacccggcagcagacg

SEQ ID NO: 71 - DNA, 42 bp, synthetic construct
  misc_feature 1..42: note = Primer
  source 1..42: mol_type = other DNA
ttgcatcaagcacgatgatcgaatgtgcagttgcagcagtcc

SEQ ID NO: 72 - DNA, 44 bp, synthetic construct
  misc_feature 1..44: note = Primer
  source 1..44: mol_type = other DNA
ggactgctgcaactgcacattcgatcatcgtgcttgatgcaatc

SEQ ID NO: 73 - DNA, 27 bp, synthetic construct
  misc_feature 1..27: note = Primer
  source 1..27: mol_type = other DNA
gcacaggcaagctttggtgcagtgatg

SEQ ID NO: 74 - DNA, 22 bp, synthetic construct
  misc_feature 1..22: note = Primer
  source 1..22: mol_type = other DNA
gcaccaaagcttgcctgtgctg

SEQ ID NO: 75 - DNA, 36 bp, synthetic construct
  misc_feature 1..36: note = Primer
  source 1..36: mol_type = other DNA
aatgacgatgatcagttgtgcagttgcagcagtccg

SEQ ID NO: 76 - DNA, 40 bp, synthetic construct
  misc_feature 1..40: note = Primer
  source 1..40: mol_type = other DNA
cggactgctgcaactgcacaactgatcatcgtcattgatg

SEQ ID NO: 77 - DNA, 23 bp, synthetic construct
  misc_feature 1..23: note = Primer
  source 1..23: mol_type = other DNA
gacgatgatccagtgtgcagttg

SEQ ID NO: 78 - DNA, 24 bp, synthetic construct
  misc_feature 1..24: note = Primer
  source 1..24: mol_type = other DNA
ctgcacactggatcatcgtcattg

SEQ ID NO: 79 - DNA, 35 bp, synthetic construct
  misc_feature 1..35: note = Primer
  source 1..35: mol_type = other DNA
aatgacgatgatctggtgtgcagttgcagcagtcc

SEQ ID NO: 80 - DNA, 35 bp, synthetic construct
  misc_feature 1..35: note = Primer
  source 1..35: mol_type = other DNA
ggactgctgcaactgcacaccagatcatcgtcatt

SEQ ID NO: 81 - DNA, 24 bp, synthetic construct
  misc_feature 1..24: note = Primer
  source 1..24: mol_type = other DNA
gccgggtctggatacctatattac

SEQ ID NO: 82 - DNA, 29 bp, synthetic construct
  misc_feature 1..29: note = Primer
  source 1..29: mol_type = other DNA
atataggtatccagacccggcagcagacg

SEQ ID NO: 83 - DNA, 24 bp, synthetic construct
  misc_feature 1..24: note = Primer
  source 1..24: mol_type = other DNA
gccgggtctggatacctatattac

SEQ ID NO: 84 - DNA, 29 bp, synthetic construct
  misc_feature 1..29: note = Primer
  source 1..29: mol_type = other DNA
atataggtatccagacccggcagcagacg

SEQ ID NO: 85 - DNA, 39 bp, synthetic construct
  misc_feature 1..39: note = Primer
  source 1..39: mol_type = other DNA
gatcatcgtgcttgatgcaatcatcatcacggtccacag

SEQ ID NO: 86 - DNA, 30 bp, synthetic construct
  misc_feature 1..30: note = Primer
  source 1..30: mol_type = other DNA
ttgcatcaagcacgatgatcagctgtgcag

SEQ ID NO: 87 - DNA, 46 bp, synthetic construct
  misc_feature 1..46: note = Primer
  source 1..46: mol_type = other DNA
tgattgcatcatggacgatgatcagttgtgcagttgcagcagtccg

SEQ ID NO: 88 - DNA, 44 bp, synthetic construct
  misc_feature 1..44: note = Primer
  source 1..44: mol_type = other DNA
cggactgctgcaactgcacaactgatcatcgtccatgatgcaat

SEQ ID NO: 89 - DNA, 34 bp, synthetic construct
  misc_feature 1..34: note = Primer
  source 1..34: mol_type = other DNA
ggtctggatacctatattacgaccgacgttatcg

SEQ ID NO: 90 - DNA, 39 bp, synthetic construct
  misc_feature 1..39: note = Primer
  source 1..39: mol_type = other DNA
aacgtcggtcgtaatataggtatccagacccggcagcag

SEQ ID NO: 91 - DNA, 12 bp, synthetic construct
  variation 12: note = n is a, c, g, or t
  source 1..12: mol_type = other DNA
gaygayggytcn

SEQ ID NO: 92 - DNA, 15 bp, synthetic construct
  variation 4: note = note = n is a, c, g, or t
  variation 7: note = note = n is a, c, g, or t
  source 1..15: mol_type = other DNA
ccancgnagttgytg

SEQ ID NO: 93 - DNA, 27 bp, synthetic construct
  misc_feature 1..27: note = Primer
  source 1..27: mol_type = other DNA
tatgacaatggtcagctgcggcgtggc

SEQ ID NO: 94 - DNA, 21 bp, synthetic construct
  misc_feature 1..21: note = Primer
  source 1..21: mol_type = other DNA
cgcagctgaccattgtcatag

SEQ ID NO: 95 - DNA, 23 bp, synthetic construct
  misc_feature 1..23: note = Primer
  source 1..23: mol_type = other DNA
gacaatggtcgaatgcggcgtgg

SEQ ID NO: 96 - DNA, 24 bp, synthetic construct
  misc_feature 1..24: note = Primer
  source 1..24: mol_type = other DNA
cgccgcattcgaccattgtcatag

SEQ ID NO: 97 - DNA, 33 bp, synthetic construct; identical to SEQ ID NO: 47
  misc_feature 1..33: note = Primer
  source 1..33: mol_type = other DNA

SEQ ID NO: 98 - DNA, 20 bp, synthetic construct
  misc_feature 1..20: note = Primer
  source 1..20: mol_type = other DNA
caccgaagctagactgtgcc

SEQ ID NO: 99 - DNA, 36 bp, synthetic construct
  misc_feature 1..36: note = Primer
  source 1..36: mol_type = other DNA
ctgcagcgtcgtagcattgcgtgctcgccaacttag

SEQ ID NO: 100 - DNA, 36 bp, synthetic construct
  misc_feature 1..36: note = Primer
  source 1..36: mol_type = other DNA
ctaagttggcgagcacgcaatgctacgacgctgcag